SEQUENCE LISTING (34 sequences)

SEQ ID NO: 1 - DNA, 1616 bp, Zea mays
  misc_feature (1588)..(1589): n is a, c, g, or t
  misc_feature (1592)..(1592): n is a, c, g, or t
attgcaagagtttgagacagcagaggacactcctcctcagaactctcccttccctcttgtaggggccaaaagggtcagagatctaggaagaagagaactagtccccaagcctaacaagaaagcaagctcttctgcaactgttggcattcgccttaatttgtatccatatcccaatggcaggcctatctctgcaacacccctgggcattcactttcggcctcctaggtatatatttatttatttatctttctattctgcagttccattcgtttttcactatacagtttgctgtagctgtgtattaaaaaacaatcccgttcgacctttcatatatatatatcacctttagtttagcctcgccaatcttttggcaaatggctgatcttttttattttcctttgcaggcaacgtcatctccttcatgaccttcctggccccgatgtaagtgacatgtagcttgttgctaataaataaatcatcacagctgatcacgtataataatcgtcaatgaatcgaaccgttaatttgctcgatccattttttatatctttgaaattatgcagaccgacgttctaccgcatctacaagagcaagtcgacggagggcttccagtcggttccgtacgtggtggccctgttcagcgccatgctgtggatcttctacgcgctgatcaagtccaacgagacgttcctcatcaccatcaacgcggccggctgcgtgatcgagaccgtctacgtggtcatgtacttcgtctacgcgaccaagaagggcaggatgttcaccgccaaaatcatgctcctcctcaacgtcggcgccttcggggccatccttctcctcacccttctcctcttcaagggtgacaagcgcgtcgtcatgcttggctggatctgcgtcggcttctccgtcagcgtcttcgtcgcgccgctcagcatcatggtaagcagcagcaccatgcatctgtcgacgatgcatgcacatggatagagatatatatatactaactagctagctaatggacgtcgcggccgttgctaatggattatttgcatgcatgcgtctacgtacgtacgtgcagaggcgggtgatccagacgaagagcgtggagtacatgcccttctccctctccctctcgctcaccctcagcgccgtcgtctggttcctctacggcctcctcatcaaggacaaatacgtcgcggtatatcatttcgtttaattgtttgcaacaccggccgtgtgatgtgtgtcctctagctactgatatcatatatccatgcagcttccaaacatccttggcttcaccttcggcgtggtccagatggtgctgtacgtggtgtacatgaacaagacgccgctgccggttgccgacggcaaggctgccggcaagcttccctcagctgcagatgagcacgtcgtcgtcaacgtaaccaagctcagcccgggccgactcccaccagtaacacagatggcggcggttcccaccaagagctgcgctacagaagcagccgcgccggcgacgctgcccagcagggacgtggtcgacgtcttggtcaaccgccacagccccgccgtccatgtggcctagctaganntcntgcccgtgcagcatgcgcccgcca

SEQ ID NO: 2 - DNA, 1026 bp, Zea mays
  misc_feature (1021)..(1021): n is a, c, g, or t
gaagaagagaactagtccccaagcctaacaagaaagcaagctcttctgcaactgttggcattcgccttaatttgtatccatatcccaatggcaggcctatctctgcaacacccctgggcattcactttcggcctcctaggcaacgtcatctccttcatgaccttcctggccccgataccgacgttctaccgcatctacaagagcaagtcgacggagggcttccagtcggttccgtacgtggtggccctgttcagcgccatgctgtggatcttctacgcgctgatcaagtccaacgagacgttcctcatcaccatcaacgcggccggctgcgtgatcgaaaccgtctacgtggtcatgtacttcgtctacgcgaccaagaagggcaggatgttcaccgccaaaatcatgctcctcctcaacgtcggcgccttcggggccatccttctcctcacccttctcctcttcaagggtgacaagcgcgtcgtcatgcttggctggatctgcgtcggcttctccgtcagcgtcttcgtcgcgccgctcagcatcatgaggcgggtgatccagaccaagagcgtggagtacatgcccttctccctctccctctcgctcaccctcagcgccgtcgtctggttcctctacggcctcctcatcaaggacaaatacgtcgcgcttccaaacatccttggcttcaccttcggcgtggtccagatggtgctgtacgtggtgtacatgaacaagacgccgctgccggttgccgacggcaaggctgccggcaagcttccctcagctgcagatgagcacgtcgtcgtcaacgtcaccaagctcagcccgggccgactcccaccagtaacacagatggcggcggttcccaccaagagctgcgctacagaagcagccgcgccggcgacgctgcccagcagggacgtggtcgacgtcttggtcaaccgccacagccccgccgtccatgtgacctagctagatctcatgcccgtgcagcatgcgcccgccatatatacggacnggtca

SEQ ID NO: 3 - DNA, 3510 bp, Zea mays
accatcaagctttcaggcagggcaagcaactcgagtagtagttgccagccacacctcctgtcttcctttttccttctcgcattgtggaggagaaggatcaagtgctcataagcaagctagcttgacgaagtataggaaattaggaatacaaataaactcatcctcctcatatataaacgctcgatctccagatatggccggcctgtccctgctgcatcccatggccttcgccttcggcctcctcggtattttttttacatttccttatttcactgtaagtgcgcttctcatcgggaaaagggtctcccctatagctaggatatacatataaaacataattttatttattcttcattattgggtcggaaggagaagtagatatatttttttttacagaaagcagggaatgagatatatatatataggacatatatgctatggtgccctgtccattatttagggcagaaagagggggaaaagaagaaaagaaaaaaggaggatatatatatatatatatttgcatgcctttgctgcctctgattaggggtggacacttttttaccataaacagaaaaccgaaccgaaatttcgatttttcgttagttcggttcggttttggtttttgtatctaagaagttcggttttcggcatcgtaatcggttttcaccgtataccgaaccgaaataccaaaaaaaccgaataccaaactttatcaattctcaaatttgactattagattatgtgaactaaatgtgtgatacaattaaattattattcacttatttgtatgtgatgtatgatgtatctctaaatatttgtgcctatataatttttatttttaaaaattatgtgtaatctatcatgtaaacttgttatatgtattgtcttaagtataagtttggtatttggtttttaccgaaaaaccaaagtaaaaaaccgaaaccgaatttctcggtttttcattttctagaaaaccgaacggtttctaatgtacgaagaaccgaagttttataaaaccgaaaaccgaaccgaagtttagaaaaaaaccgaatgcccagccctgatggtgtggcgtgacatcacaaagcgtcgtcgtcatcgaaattgattgaaggagatagttgcattgcatgtgtcatgctcatgcacagagctcgagctccccagctgaattctatctgacgcctctcgctcctttttcttttgtttggcggtttgctgtttgtgtttgcaggcaacatcatctccttcatgacctacctggccccactgtaaggctctctccctctctctctagactctagctcatcagctgcatatgtgcgcgtgatggtctgctgaccgcgtggtttggcatgggggcgcatcgtccatgggacgatgacgacgacgatgacgacgacgcaggccgacgttctaccggatctacaagaacaagtcgacggaggggttccagtcggtgccgtacgtggtggccctcttcagcgccatgctgtggatctactacgcgctgctcaagtccaacgagctcctgctcatcaccatcaactccgccggctgcgtcatcgagaccctctacatcgccatgtacctcctctacgcgcccaagaaggcaaaggtccgtcgtcgccgtcgtgcttctcattcctcctgctttgggccggccgggattaattcggccgggcatgcaaagctcatccatgcacatgcatccgtcctcacccacgcacgcgcatacgtatatatgcagctgttcacggccaagatcctcctcctcctcaacgtgggcgtgttcgggctcatcctcctcctcacgctgctgctctccgccggacagcgccgcgtcgtcgtcctcggctgggtctgcgtcgccttctccgttagcgtcttcgtcgcgccgctcagcatcatcgtgcgtacactacccaacaccatgcatatatatatctagctacctatcgagtgtatagatgcttattgcaacctctaacaccatgcatatacatggctgctgcagcgccaggtggtgaggacaaggagcgtcgagttcatgcccttctcgctctccctctccctcaccgtcagcgccgtcgtctggttcctctacggcctcctcatcaaggacaaatacgtcgctgtaagtaaactagagctttcatttcggcatgtaaaagagccgcgagataacttgactcgatcagaggcggatccagagggggggcaaagtgggccatggccccccctcaattttgtacactcttttatacctaataattatttagtattaagtgtgggaaaaatcaaaattttagatagtataaagtcttgttctaaatctctaaaactaatagctagttgctaaaattagccgagaggtttagaatggatcaactaattgttagctatactcctcaaataagtattaattgttagatatcccaacccagctaaaaccagttaacagttagctctagaggtttagaacatgacctaaaacaatcatattatgagcctgctcgccgaatcctctcggttcagtccttgcacctaccagtctaactctaatcttcccgacaccacctaaatcagtctgctcgctgccctgtttacgccgccgacccattcccgatgcgacgccaccggacttcgccccttcctagcgcctccgtcacttgtcctgtttttctcgtgacgtcgatccgctcgacaagctagatctcgctcgtcgcaacgatatgacttatctcatgcggtcagtggacttagcgaattagaaaatcatagctcccatgtggtttttttttggccccccctaaattttcgttctgcgtccgccactggactcgatcgtcccctttctccagcgaaacagatcgatcgatcagcaactgacagctctttctattttgtttgcgtcctcatcagctgccaaacgtcatcggcttcagcttcggcgtggtccagatgggtctctacgcgctctaccgcaacgcgacgcccagggtgcctgccaaggacgtggccgacgacgccagcaaggacaaggcgcccggcgaacatgtggtggtcaccattgccaagctgaccgcggcgacgacggcacctgcagctgccgtggcggaggatcttgtcaaggtccacgacggccacccggaggaagcagccaagggcgccgccaagccggcggagaacggcgcgggacgcagcgacgccgagcaagtctagattcctcgctcgatcagatcagaattcatgaagaagaacatgcgtatgtatgtgtatccggtaggtcgtaacataacataaattaagtgcgcatgcagtttggcaactcatgttgtttcatttcgttctcgcgcaatatgcgcatgtattaattttagattgtcattgttgtagttcttgcctccgattattattagtgtgtccgtcaccttccatgtaataacaaaaaaaatatatataaatggtcctattaaagcaaaagctaaagttcgtttttatgccaaa

SEQ ID NO: 4 - DNA, 1360 bp, Zea mays
accatcaagctttcaggcagggcaagcaactcgagtagtagttgccagccacacctcctgtcttcctttttccttctcgcattgtggaggagaaggatcaagtgctcataagcaagctagcttgacgaagtataggaaattaggaatacaaataaactcatcctcctcatatataaacgctcgatctccagatatggccggcctgtccctgctgcatcccatggccttcgccttcggcctcctcggcaacatcatctccttcatgacctacctggccccactgccgacgttctaccggatctacaagaacaagtcgacggaggggttccagtcggtgccgtacgtggtggccctcttcagcgccatgctgtggatctactacgcgctgctcaagtccaacgagctcctgctcatcaccatcaactccgccggctgcgtcatcgagaccctctacatcgccatgtacctcctctacgcgcccaagaaggcaaagctgttcacggccaagatcctcctcctcctcaacgtgggcgtgttcgggctcatcctcctcctcacgctgctgctctccgccggacagcgccgcgtcgtcgtcctcggctgggtctgcgtcgccttctccgttagcgtcttcgtcgcgccgctcagcatcatccgccaggtggtgaggacaaggagcgtcgagttcatgcccttctcgctctccctctccctcaccgtcagcgccgtcgtctggttcctctacggcctcctcatcaaggacaaatacgtcgctctgccaaacgtcatcggcttcagcttcggcgtggtccagatgggtctctacgcgctctaccgcaacgcgacgcccagggtgcctgccaaggacgtggccgacgacgccagcaaggacaaggcgcccggcgaacatgtggtggtcaccattgccaagctgaccgcggcgacgacggcacctgcagctgccgtggcggaggatcttgtcaaggtccacgacggccacccggaggaagcagccaagggcgccgccaagccggcggagaacggcgcgggacgcagcgacgccgagcaagtctagattcctcgctcgatcagatcagaattcatgaagaagaacatgcgtatgtatgtgtatccggtaggtcgtaacataacataaattaagtgcgcatgcagtttggcaactcatgttgtttcatttcgttctcgcgcaatatgcgcatgtattaattttagattgtcattgttgtagttcttgcctccgattattattagtgtgtccgtcaccttccatgtaataacaaaaaaaatatatataaatggtcctattaaagcaaaagctaaagttcgtttttatgccaaa

SEQ ID NO: 5 - DNA, 882 bp, Zea mays
atggccggcctgtccctgctgcatcccatggccttcgccttcggcctcctcggcaacatcatctccttcatgacctacctggccccactgccgacgttctaccggatctacaagaacaagtcgacggaggggttccagtcggtgccgtacgtggtggccctcttcagcgccatgctgtggatctactacgcgctgctcaagtccaacgagctcctgctcatcaccatcaactccgccggctgcgtcatcgagaccctctacatcgccatgtacctcctctacgcgcccaagaaggcaaagctgttcacggccaagatcctcctcctcctcaacgtgggcgtgttcgggctcatcctcctcctcacgctgctgctctccgccggacagcgccgcgtcgtcgtcctcggctgggtctgcgtcgccttctccgttagcgtcttcgtcgcgccgctcagcatcatccgccaggtggtgaggacaaggagcgtcgagttcatgcccttctcgctctccctctccctcaccgtcagcgccgtcgtctggttcctctacggcctcctcatcaaggacaaatacgtcgctctgccaaacgtcatcggcttcagcttcggcgtggtccagatgggtctctacgcgctctaccgcaacgcgacgcccagggtgcctgccaaggacgtggccgacgacgccagcaaggacaaggcgcccggcgaacatgtggtggtcaccattgccaagctgaccgcggcgacgacggcacctgcagctgccgtggcggaggatcttgtcaaggtccacgacggccacccggaggaagcagccaagggcgccgccaagccggcggagaacggcgcgggacgcagcgacgccgagcaagtctag

SEQ ID NO: 6 - DNA, 2656 bp, Zea mays
gcctatataaagccacccacagccctgcctatcattgcaagagtttgagccaacacacacagagagaggacaactcctcacaactctcccttccctcctgtaggggccaaaagggttagagagtaggagaagtagttccctagcccaacacaagaaaaacaagctcgatctcctcatcaccctaatccaagcaactgctttgtgtgttgggaaattcttgtgacccctgttatattattatccgtgtagctactagcttcattcccccctttgtacccatcaaatggctggcatgtctctgcaacacccctgggcgtttgctttcggtctactaggtacatactacaccttcactaatagctaaacaagcgcccgccgcaaagctatgaattaagggcaggcatgtttggtttgctacctattttaccatactttgtctaacttttctgtctaaggttatagttcttcaattcgaacgattaatcttagccaaagtgtgacatggttagccacgaactaagcaggcccttaattattgcgcatgtatatattatatatttatctttctattctgtttaatttgttttctctttacatatatatactactatgtaagtatatattatatgcacaacaagcaggcctccttcgtgcacatatgcatagaatcaatctatacccttcatagaagccactttgagatataccttccaaaacaatcccaaaaacaagacgctcgatcttgcgctcacaatcacttagttttgcaccagattaagcatgcaccactagattttatgtactgtattacctctgccatccatggtcgatcctttagtttatcctattcatttccgtcatgaactacctgtcgagctagcaatcggtcctttatttagagtgttcagataggcatctgtctttatacaaacaataaagcctcacgaatctttagtcacaaaacaaggcaaattagacaggccacggaggtgtaaagtgtcagctctgcttatcacaacttatctctgctttatttgggcacacttttgccatacaaatggctgatcttggcgccttttttttctccttgctttgcaggtaacgtcatctccttcatgaccttcctggccccgatgtaagtgacatatatatatatatatattgcttaattaattatcactgcttcttcagatatatattcatcggttattttaattaattatgtggatatgtatgcatcgtataacagaccgacgttctaccgcatctacaagagcaagtcgacggaaggcttccagtcggttccctacgtggttgccctgttcagcgccatgctgtggatcttctacgcactgatcaagtccaacgagaccttcctcatcaccatcaacgccgccggctgcgtcatcgagaccatctacgtcgtcatgtacttcgtctacgcgcccaagaaagccaagctgttcacggccaagatcatggtcctcctcaatggcggcgtctttggggtcatcctcctgctcacccttctcctcttcaagggcagtaagcgcgttgtgctgcttggctggatctgcgtcggcttctccgtcagtgtcttcgtcgcgccactcagcatcatggtgagccctgagcacgcgtataaaactgtgccaagatgcatggacgacagatcgatcaacccaatcagttttgatccatgtgtatcgtttctaatgcaccgtgtttatatatgtgtgcagagacgagtgatccagacgaagagcgtggagtacatgcccttctccctctccctctcgctcaccctcagcgccgtcgtctggttcctctacggcctcctcatcaaggacaaatacgtcgcggtaattgtttcatctaatctgctgcaaccgccatggtattggtatctctcactggtctttactgataaactacatacgatctctgtacgtatgcagcttccaaacatcctggggttcaccttcggcgtggtccagatggtgctctacgtgttgtacatgaacaagacgccggtggcggcgactgccgagggcaaggatgccggcaagctttcctcagctgcagacgagcacgtcctcgtcaacatcgccaagctcagcccagccctcccggagaggagctccggggtgcacccagtcacccagatggcgggcgttcctgtcaggagctgcgctgctgaagcaaccgcgccggcgatgctgcccaacagggacgtggtcgacgtcttcgtcagccgacacagccccgccgtccacgtggcatagattctcgatcgatcgcgtgcatggcccatgcatgcgcccgccacacgtacgctagcttttatatattcgaaggacgacttgctgctggtcgtgagcatatatatgatggagaaaatgattaagtagtatatatataagtaattaactgccatgcatggaagctagctaatggatggaggcagaggccagaacgatgaagggggaagctatacatatatgtgtgtaattaatatagatatatgggctttgtgttcatctttgcagctatgtattaatttgcatggatatctgttattcctttttatgtgtaacgtcttctaataaaatgtaattgaacccacactactgttgttagctaccacaagtttccc

SEQ ID NO: 7 - DNA, 2656 bp, Zea mays; identical to SEQ ID NO: 6

SEQ ID NO: 8 - DNA, 909 bp, Zea mays
atggctggcatgtctctgcaacacccctgggcgtttgctttcggtctactaggtaacgtcatctccttcatgaccttcctggccccgataccgacgttctaccgcatctacaagagcaagtcgacggaaggcttccagtcggttccctacgtggttgccctgttcagcgccatgctgtggatcttctacgcactgatcaagtccaacgagaccttcctcatcaccatcaacgccgccggctgcgtcatcgagaccatctacgtcgtcatgtacttcgtctacgcgcccaagaaagccaagctgttcacggccaagatcatggtcctcctcaatggcggcgtctttggggtcatcctcctgctcacccttctcctcttcaagggcagtaagcgcgttgtgctgcttggctggatctgcgtcggcttctccgtcagtgtcttcgtcgcgccactcagcatcatgagacgagtgatccagacgaagagcgtggagtacatgcccttctccctctccctctcgctcaccctcagcgccgtcgtctggttcctctacggcctcctcatcaaggacaaatacgtcgcgcttccaaacatcctggggttcaccttcggcgtggtccagatggtgctctacgtgttgtacatgaacaagacgccggtggcggcgactgccgagggcaaggatgccggcaagctttcctcagctgcagacgagcacgtcctcgtcaacatcgccaagctcagcccagccctcccggagaggagctccggggtgcacccagtcacccagatggcgggcgttcctgtcaggagctgcgctgctgaagcaaccgcgccggcgatgctgcccaacagggacgtggtcgacgtcttcgtcagccgacacagccccgccgtccacgtggcatag

SEQ ID NO: 9 - DNA, 1270 bp, Zea mays
attgcaagagtttgagacagcagaggacactcctcctcagaactctcccttccctcttgtaggggccaaaagggtcagagatctaggaagaagagaactagtccccaagcctaacaagaaagcaagctcttctgcaactgttggcattcgccttaatttgtatccatatcccaatggcaggcctatctctgcaacacccctgggcattcactttcggcctcctaggtatatatttatttatttatctttctattctgcagttccattcgtttttcactatacagtttgctgtagctgtgtattaaaaaacaatcccgttcgacctttcatatatatatatcacctttagtttagcctcgccaatcttttggcaaatggctgatcttttttattttcctttgcaggcaacgtcatctccttcatgaccttcctggccccgatgtaagtgacatgtagcttgttgctaataaataaatcatcacagctgatcacgtataataatcgtcaatgaatcgaaccgttaatttgctcgatccattttttatatctttgaaattatgcagaccgacgttctaccgcatctacaagagcaagtcgacggagggcttccagtcggttccgtacgtggtggccctgttcagcgccatgctgtggatcttctacgcgctgatcaagtccaacgagacgttcctcatcaccatcaacgcggccggctgcgtgatcgagaccgtctacgtggtcatgtacttcgtctacgcgaccaagaagggcaggatgttcaccgccaaaatcatgctcctcctcaacgtcggcgccttcggggccatccttctcctcacccttctcctcttcaagggtgacaagcgcgtcgtcatgcttggctggatctgcgtcggcttctccgtcagcgtcttcgtcgcgccgctcagcatcatggtaagcagcagcaccatgcatctgtcgtcgatgcatgcatgcacatggatatatatatataccagcagggacgtggtcgacgtcttggtcaaccgccacagcccgccgtccatgtgacctagctagatctcatgcccgtgcagcatgcgcccgccatatatacggactggtcatgagcatatgatggaattataagtaattaattgccatgtaagctagctaatgatgcatggagacataggtcagaggtagagggccaagcgcgctctctctctctctctatctatctatatctatttttatatctatatagagagtctatattcatgtttaa

SEQ ID NO: 10 - DNA, 656 bp, Zea mays
attgcaagagtttgagacagcagaggacactcctcctcagaactctcccttccctcttgtaggggccaaaagggtcagagatctaggaagaagagaactagtccccaagcctaacaagaaagcaagctcttctgcaactgttggcattcgccttaatttgtatccatatcccaatggcaggcctatctctgcaacacccctgggcattcactttcggcctcctaggcaacgtcatctccttcatgaccttcctggccccgataccgacgttctaccgcatctacaagagcaagtcgacggagggcttccagtcggttccgtacgtggtggccctgttcagcgccatgctgtggatcttctacgcgctgatcaagtccaacgagacgttcctcatcaccatcaacgcggccggctgcgtgatcgagaccgtctacgtggtcatgtacttcgtctacgcgaccaagaagggcaggatgttcaccgccaaaatcatgctcctcctcaacgtcggcgccttcggggccatccttctcctcacccttctcctcttcaagggtgacaagcgcgtcgtcatgcttggctggatctgcgtcggcttctccgtcagcgtcttcgtcgcgccgctcagcatcatgagagtctatattcatgtttaa

SEQ ID NO: 11 - DNA, 1261 bp, Zea mays
aattgcaagagtttgagacagcagaggacactcctcctcagaactctcccttccctcttgtaggggccaaaagggtcagagatctaggaagaagagaactagtccccaagcctaacaagaaagcaagctcttctgcaactgttggcattcgccttaatttgtatccatatcccaatggcaggcctatctctgcaacacccctgggcattcactttcggcctcctaggcaacgtcatctccttcatgaccttcctggccccgataccgacgttctaccgcatctacaagagcaagtcgacggagggcttccagtcggttccgtacgtggtggccctgttcagcgccatgctgtggatcttctacgcgctgatcaagtccaacgagacgttcctcatcaccatcaacgcggccggctgcgtgatcgagaccgtctacgtggtcatgtacttcgtctacgcgaccaagaagggcaggatgttcaccgccaaaatcatgctcctcctcaacgtcggcgccttcgggtccatcctcctcctcacccttctcctcttcaagggtgacaagcgcgtcgtcatgcttggctggatctgcgtcggcttctccgtcagcgtcttcgtcgcgccgctcagcatcatgaggcgggtgatccagaccaagagcgtggagtacatgcccttctccctctccctctcgctcaccctcagcgccgtcgtctggttcctctacggcctcctcatcaaggacaaatacgtcgcgcttccaaacatccttggcttcaccttcggcgtggtccagatggtgctgtacgtggtgtacatgaacaagacgccgctgccggttgccgacggcaaggctgccggcaagcttccctcagctgcagatgagcacgtcgtcgtcaacgtcaccaagctcagcccgggccgactcccaccagtaacacagatggcggcggttcccaccaagagctgcgctacagaagcagccgcgccggcgacgctgcccagcagggacgtggtcgacgtcttggtcaaccgccacagccccgccgtccatgtgacctagctagatctcatgcccgtgcagcatgcgcccgccatatatacggactggtcatgagcatatgatggaattataagtaattaattgccatgtaagctagctaatgatgcatggagacataggtcagaggtagagggccaagcgcgcgctctctctctctatctatctatatctatttttatatcaaaaaaaaaaaaaaaaa

SEQ ID NO: 12 - protein, 295 aa, Zea mays
MAGLSLQHPWAFTFGLLGNVISFMTFLAPIPTFYRIYKSKSTEGFQSVPYVVALFSAMLWIFYALIKSNETFLITINAAGCVIETVYVVMYFVYATKKGRMFTAKIMLLLNVGAFGAILLLTLLLFKGDKRVVMLGWICVGFSVSVFVAPLSIMRRVIQTKSVEYMPFSLSLSLTLSAVVWFLYGLLIKDKYVALPNILGFTFGVVQMVLYVVYMNKTPLPVADGKAAGKLPSAADEHVVVNVTKLSPGRLPPVTQMAAVPTKSCATEAAAPATLPSRDVVDVLVNRHSPAVHVT

SEQ ID NO: 13 - protein, 293 aa, Zea mays
MAGLSLLHPMAFAFGLLGNIISFMTYLAPLPTFYRIYKNKSTEGFQSVPYVVALFSAMLWIYYALLKSNELLLITINSAGCVIETLYIAMYLLYAPKKAKLFTAKILLLLNVGVFGLILLLTLLLSAGQRRVVVLGWVCVAFSVSVFVAPLSIIRQVVRTRSVEFMPFSLSLSLTVSAVVWFLYGLLIKDKYVALPNVIGFSFGVVQMGLYALYRNATPRVPAKDVADDASKDKAPGEHVVVTIAKLTAATTAPAAAVAEDLVKVHDGHPEEAAKGAAKPAENGAGRSDAEQV

SEQ ID NO: 14 - protein, 302 aa, Zea mays
MAGMSLQHPWAFAFGLLGNVISFMTFLAPIPTFYRIYKSKSTEGFQSVPYVVALFSAMLWIFYALIKSNETFLITINAAGCVIETIYVVMYFVYAPKKAKLFTAKIMVLLNGGVFGVILLLTLLLFKGSKRVVLLGWICVGFSVSVFVAPLSIMRRVIQTKSVEYMPFSLSLSLTLSAVVWFLYGLLIKDKYVALPNILGFTFGVVQMVLYVLYMNKTPVAATAEGKDAGKLSSAADEHVLVNIAKLSPALPERSSGVHPVTQMAGVPVRSCAAEATAPAMLPNRDVVDVFVSRHSPAVHVA

SEQ ID NO: 15 - protein, 160 aa, Zea mays
MAGLSLQHPWAFTFGLLGNVISFMTFLAPIPTFYRIYKSKSTEGFQSVPYVVALFSAMLWIFYALIKSNETFLITINAAGCVIETVYVVMYFVYATKKGRMFTAKIMLLLNVGAFGAILLLTLLLFKGDKRVVMLGWICVGFSVSVFVAPLSIMRVYIHV

SEQ ID NO: 16 - protein, 295 aa, Zea mays; identical to SEQ ID NO: 12

SEQ ID NO: 17 - DNA, 1116 bp, Oryza sativa
atggatttgagcaatagctcacctgtcatcaccgatccggtggcgatcagccagcagttgttgggcggcctgccttcaaatctgatgcagttttcagtcatgcccggtggctactccagctctggcatgaacgttggtgtcagtaggctcaaaatcgaggaagtccttgtcaatggactgcttgatgccatgaaatcctcgtcacctcgcaggaggctgaatgtagcatttggcgaggacaattcatctgaagaagaagaccctgcttacagcgcttggatggcaaaatgtccttctgctttggcttccttcaagcaaattgtagccagtgcacaagggaagaagattgctgtgtttctagactatgacggcacactgtcgcctattgtggatgatcctgacaaagcagtgatgtctcccgtgatgagagctgctgtgagaaatgttgcgaagtacttccccactgcaattgtcagcggaaggtcccgcaataaggtgtttgaatttgtaaaactgaaggagctttattatgctggaagtcatggtatggacataatggcaccttcagcaaatcatgagcacagtgctgaaaagagcaaacaggccaatctcttccaacctgcacacgactttctgccaatgatcgatgaggttaccaagtccctcttgcaagttgtcagtggaattgaaggtgcaactgttgagaacaacaaattctgcgtttctgtacattatcgcaacgttgcagagaaggattggaaactggtcgcacggctcgtaaacgaagtgctggaggcttttcctcgtctcaaagtaaccaatggacgaatggttttagaggttcgtccggtgatcgactgggacaagggaaaggctgtggagtttctgctccagtcactcgggctaaatgactctgaaaatgtgatccccatctacattggagacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtga

SEQ ID NO: 18 - DNA, 948 bp, Oryza sativa
gtcaatggactgcttgatgccatgaaatcctcgtcacctcgcaggaggctgaatgtagcatttggcgaggacaattcatctgaagaagaagaccctgcttacagcgcttggatggcaaaatgtccttctgctttggcttccttcaagcaaattgtagccagtgcacaagggaagaagattgctgtgtttctagactatgacggcacactgtcgcctattgtggatgatcctgacaaagcagtgatgtctcccgtgatgagagctgctgtgagaaatgttgcgaagtacttccccactgcaattgtcagcggaaggtcccgcaataaggtgtttgaatttgtaaaactgaaggagctttattatgctggaagtcatggtatggacataatggcaccttcagcaaatcatgagcacagtgctgaaaagagcaaacaggccaatctcttccaacctgcacacgactttctgccaatgatcgatgaggttaccaagtccctcttgcaagttgtcagtggaattgaaggtgcaactgttgagaacaacaaattctgcgtttctgtacattatcgcaacgttgcagagaaggattggaaactggtcgcacggctcgtaaacgaagtgctggaggcttttcctcgtctcaaagtaaccaatggacgaatggttttagaggttcgtccggtgatcgactgggacaagggaaaggctgtggagtttctgctccagtcactcgggctaaatgactctgaaaatgtgatccccatctacattggagacgacagaactgacgaagacgctttcaaggtacttcgacagcgaaattgcggttatggaatactagtttcacaggttcccaaggaaactgaagccttctactcgctgagagatccatctgaagtgatggagttcctcaatttcttggtgagatggaagaagcactcagtgtga

SEQ ID NO: 19 - DNA, 1113 bp, Oryza sativa
atggatctgagcaacagcagcccggtgattaccgatccggtggcgattagccagcagctgctgggcggcctgccgagcaacctgatgcagtttagcgtgatgccgggcggctatagcagcagcggcatgaacgtgggcgtgagccgcctgaaaattgaagaagtgctggtgaacggcctgctggatgcgatgaaaagcagcagcccgcgccgccgcctgaacgtggcgtttggcgaagataacagcagcgaagaagaagatccggcgtatagcgcgtggatggcgaaatgcccgagcgcgctggcgagctttaaacagattgtggcgagcgcgcagggcaaaaaaattgcggtgtttctggattatgatggcaccctgagcccgattgtggatgatccggataaagcggtgatgagcccggtgatgcgcgcggcggtgcgcaacgtggcgaaatattttccgaccgcgattgtgagcggccgcagccgcaacaaagtgtttgaatttgtgaaactgaaagaactgtattatgcgggcagccatggcatggatattatggcgccgagcgcgaaccatgaacatagcgcggaaaaaagcaaacaggcgaacctgtttcagccggcgcatgattttctgccgatgattgatgaagtgaccaaaagcctgctgcaggtggtgagcggcattgaaggcgcgaccgtggaaaacaacaaattttgcgtgagcgtggattatcgcaacgtggcggaaaaagattggaaactggtggcgcgcctggtgaacgaagtgctggaagcgtttccgcgcctgaaagtgaccaacggccgcatggtgctggaagtgcgcccggtgattgattgggataaaggcaaagcggtggaatttctgctgcagagcctgggcctgaacgatagcgaaaacgtgattccgatttatattggcgatgatcgcaccgatgaagatgcgtttaaagtgctgcgccagcgcaactgcggctatggcattctggtgagccaggtgccgaaagaaaccgaagcgttttatagcctgcgcgatccgagcgaagtgatggaatttctgaactttctggtgcgctggaaaaaacatagcgtg

SEQ ID NO: 20 - DNA, 1113 bp, Oryza sativa
atggatctgagcaacagcagcccggtgattaccgatccggtggcgattagccagcagctgctgggcggcctgccgagcaacctgatgcagtttagcgtgatgccgggcggctatagcagcagcggcatgaacgtgggcgtgagccgcctgaaaattgaagaagtgctggtgaacggcctgctggatgcgatgaaaagcagcagcccgcgccgccgcctgaacgtggcgtttggcgaagataacagcagcgaagaagaagatccggcgtatagcgcgtggatggcgaaatgcccgagcgcgctggtgagctttaaacagattgtggcgagcgcgcagggcaaaaaaattgcggtgtttctggattatgatggcaccctgagcccgtttgtggatgatccggataaagcggtgatgagcccggtgatgcgcgcggcggtgcgcaacgtggcgaaatattttccgaccgcgattgtgagcggccgcagccgcaacaaagtgtttgaatttgtgaaactgaaagaactgtattatgcgggcagccatggcatggatattatggcgccgagcgcgaaccatgaacatagcgcggaaaaaagcaaacaggcgaacctgtttcagccggcgcatgattttctgccgatgattgatgaagtgaccaaaagcctgctgcaggtggtgagcggcattgaaggcgcgaccgtggaaaacaacaaattttgcgtgagcgtgcattatcgcaacgtggcggaaaaagattggaaactggtggcgcgcctggtgaacgaagtgctggaagcgtttccgcgcctgaaagtgaccaacggccgcatggtgctggaagtgcgcccggtgattgattgggataaaggcaaagcggtggaatttctgctgcagagcctgggcctgaacgatagcgaaaacgtgattccgatttatattggcgatgatcgcaccgatgaagatgcgtttaaagtgctgcgccagcgcaactgcggctatggcattctggtgagccaggtgccgaaagaaaccgaagcgttttatagcctgcgcgatccgagcgaagtgatggaatttctgaactttctggtgcgctggaaaaaacatagcgtg

SEQ ID NO: 21 - protein, 371 aa, Oryza sativa
MDLSNSSPVITDPVAISQQLLGGLPSNLMQFSVMPGGYSSSGMNVGVSRLKIEEVLVNGLLDAMKSSSPRRRLNVAFGEDNSSEEEDPAYSAWMAKCPSALASFKQIVASAQGKKIAVFLDYDGTLSPIVDDPDKAVMSPVMRAAVRNVAKYFPTAIVSGRSRNKVFEFVKLKELYYAGSHGMDIMAPSANHEHSAEKSKQANLFQPAHDFLPMIDEVTKSLLQVVSGIEGATVENNKFCVSVHYRNVAEKDWKLVARLVNEVLEAFPRLKVTNGRMVLEVRPVIDWDKGKAVEFLLQSLGLNDSENVIPIYIGDDRTDEDAFKVLRQRNCGYGILVSQVPKETEAFYSLRDPSEVMEFLNFLVRWKKHSV

SEQ ID NO: 22 - protein, 315 aa, Oryza sativa
VNGLLDAMKSSSPRRRLNVAFGEDNSSEEEDPAYSAWMAKCPSALASFKQIVASAQGKKIAVFLDYDGTLSPIVDDPDKAVMSPVMRAAVRNVAKYFPTAIVSGRSRNKVFEFVKLKELYYAGSHGMDIMAPSANHEHSAEKSKQANLFQPAHDFLPMIDEVTKSLLQVVSGIEGATVENNKFCVSVHYRNVAEKDWKLVARLVNEVLEAFPRLKVTNGRMVLEVRPVIDWDKGKAVEFLLQSLGLNDSENVIPIYIGDDRTDEDAFKVLRQRNCGYGILVSQVPKETEAFYSLRDPSEVMEFLNFLVRWKKHSV

SEQ ID NO: 23 - protein, 371 aa, Oryza sativa
MDLSNSSPVITDPVAISQQLLGGLPSNLMQFSVMPGGYSSSGMNVGVSRLKIEEVLVNGLLDAMKSSSPRRRLNVAFGEDNSSEEEDPAYSAWMAKCPSALASFKQIVASAQGKKIAVFLDYDGTLSPIVDDPDKAVMSPVMRAAVRNVAKYFPTAIVSGRSRNKVFEFVKLKELYYAGSHGMDIMAPSANHEHSAEKSKQANLFQPAHDFLPMIDEVTKSLLQVVSGIEGATVENNKFCVSVDYRNVAEKDWKLVARLVNEVLEAFPRLKVTNGRMVLEVRPVIDWDKGKAVEFLLQSLGLNDSENVIPIYIGDDRTDEDAFKVLRQRNCGYGILVSQVPKETEAFYSLRDPSEVMEFLNFLVRWKKHSV

SEQ ID NO: 24 - protein, 371 aa, Oryza sativa
MDLSNSSPVITDPVAISQQLLGGLPSNLMQFSVMPGGYSSSGMNVGVSRLKIEEVLVNGLLDAMKSSSPRRRLNVAFGEDNSSEEEDPAYSAWMAKCPSALVSFKQIVASAQGKKIAVFLDYDGTLSPFVDDPDKAVMSPVMRAAVRNVAKYFPTAIVSGRSRNKVFEFVKLKELYYAGSHGMDIMAPSANHEHSAEKSKQANLFQPAHDFLPMIDEVTKSLLQVVSGIEGATVENNKFCVSVHYRNVAEKDWKLVARLVNEVLEAFPRLKVTNGRMVLEVRPVIDWDKGKAVEFLLQSLGLNDSENVIPIYIGDDRTDEDAFKVLRQRNCGYGILVSQVPKETEAFYSLRDPSEVMEFLNFLVRWKKHSV

SEQ ID NO: 25 - protein, 26 aa, Artificial Sequence
  Synthetic - Clade III transporter domain
  MISC_FEATURE (1)..(1): Xaa can be V or M
  MISC_FEATURE (2)..(2): Xaa can be F or Y
  MISC_FEATURE (3)..(3): Xaa can be V/A/G/S
  MISC_FEATURE (4)..(4): Xaa can be A or S
  MISC_FEATURE (6)..(6): Xaa can be L/S/M
  MISC_FEATURE (7)..(7): Xaa can be any naturally occurring amino acid
  MISC_FEATURE (8)..(8): Xaa can be I/V/A
  MISC_FEATURE (9)..(9): Xaa can be M/I/L/V
  MISC_FEATURE (10)..(11): Xaa can be any naturally occurring amino acid
  MISC_FEATURE (13)..(13): Xaa can be I or V
  MISC_FEATURE (14)..(14): Xaa can be K or X
  MISC_FEATURE (15)..(15): Xaa can be T or S
  MISC_FEATURE (16)..(16): Xaa can be K/R/E
  MISC_FEATURE (18)..(18): Xaa can be V or A
  MISC_FEATURE (19)..(19): Xaa can be E/K/Q/A
  MISC_FEATURE (20)..(20): Xaa can be Y or F
  MISC_FEATURE (24)..(24): Xaa can be any naturally occurring amino acid
  MISC_FEATURE (25)..(25): Xaa can be L or M
XXXXPXXXXXXVXXXXSXXXMPFXXS

SEQ ID NO: 26 - protein, 28 aa, Artificial Sequence
  Synthetic - Clade III transporter domain
  MISC_FEATURE (1)..(1): Xaa can be K/R/A
  MISC_FEATURE (2)..(2): Xaa can be K/N/S
  MISC_FEATURE (4)..(4): Xaa can be S/T
  MISC_FEATURE (5)..(5): Xaa can be T/S/I/A
  MISC_FEATURE (6)..(6): Xaa can be E/K/Q/G
  MISC_FEATURE (7)..(7): Xaa can be G/S/C
  MISC_FEATURE (8)..(8): Xaa can be F/Y
  MISC_FEATURE (9)..(9): Xaa can be Q/S/E/H
  MISC_FEATURE (10)..(10): Xaa can be S/A
  MISC_FEATURE (11)..(11): Xaa can be I/L/V/T
  MISC_FEATURE (14)..(14): Xaa can be any naturally occurring amino acid
  MISC_FEATURE (15)..(15): Xaa can be V/S/T/C
  MISC_FEATURE (16)..(16): Xaa can be A/S/T
  MISC_FEATURE (18)..(18): Xaa can be F/L/A
  misc_feature (19)..(19): Xaa can be any naturally occurring amino acid
  MISC_FEATURE (20)..(20): Xaa can be A/C/S/T
  MISC_FEATURE (21)..(21): Xaa can be M/T/G/L
  misc_feature (22)..(22): Xaa can be any naturally occurring amino acid
  MISC_FEATURE (23)..(23): Xaa can be W/F/L
  MISC_FEATURE (24)..(24): Xaa can be any naturally occurring amino acid
  MISC_FEATURE (25)..(25): Xaa can be Y/F/L
  misc_feature (26)..(26): Xaa can be any naturally occurring amino acid
  MISC_FEATURE (27)..(27): Xaa can be A/G
  MISC_FEATURE (28)..(28): Xaa can be any naturally occurring amino acid
  MISC_FEATURE (29)..(29): Xaa can be K/R/Q/S/T
XXKXXXXXXXXPYXXXLSXXLXXXYXXX

SEQ ID NO: 27 - protein, 26 aa, Artificial Sequence
  Synthetic - Clade III transporter domain
  MISC_FEATURE (1)..(1): Xaa can be V/M
  MISC_FEATURE (3)..(3): Xaa can be V/A
  MISC_FEATURE (4)..(4): Xaa can be A/S
  MISC_FEATURE (7)..(7): Xaa can be S/A/F/M/T
  MISC_FEATURE (8)..(8): Xaa can be I/V
  MISC_FEATURE (9)..(9): Xaa can be I/M/V
  MISC_FEATURE (10)..(11): Xaa can be any naturally occurring amino acid
  MISC_FEATURE (13)..(13): Xaa can be I/V/M
  MISC_FEATURE (14)..(14): Xaa can be K/R/Q
  MISC_FEATURE (15)..(15): Xaa can be T/S
  MISC_FEATURE (16)..(16): Xaa can be K/R
  MISC_FEATURE (19)..(19): Xaa can be E/A
  MISC_FEATURE (20)..(20): Xaa can be Y/F
  MISC_FEATURE (21)..(21): Xaa can be M/L
  MISC_FEATURE (23)..(23): Xaa can be F/I
  MISC_FEATURE (24)..(24): Xaa can be any naturally occuring amino acid
XFXXPLXXXXXVXXXXSVXXXPXXLS

SEQ ID NO: 28 - protein, 19 aa, Artificial Sequence
  Synthetic - Clade III transporter domain
  MISC_FEATURE (3)..(3): Xaa can be V/I/G/T
  MISC_FEATURE (4)..(4): Xaa can be L/G/V/I
  MISC_FEATURE (6)..(6): Xaa can be F/L/A
  MISC_FEATURE (7)..(7): Xaa can be any naturally occurring amino acid
  MISC_FEATURE (8)..(8): Xaa can be F/L
  MISC_FEATURE (9)..(9): Xaa can be G/S
  MISC_FEATURE (10)..(11): Xaa can be any naturally occurring amino acid
  MISC_FEATURE (14)..(15): Xaa can be any naturally occurring amino acid
  MISC_FEATURE (16)..(16): Xaa can be Y/F
  MISC_FEATURE (17)..(18): Xaa can be any naturally occurring amino acid
  MISC_FEATURE (19)..(19): Xaa can be Y/F
PNXXGXXXXXXQMXXXXXX

SEQ ID NO: 29 - DNA, 3189 bp, Zea mays
ctcaggagctaatgactcacctctccgtgctcacctcctcttcttgaggcttgagctctctcttcctctctcccctcagctcatctccaccgtctccctatatataggatgctctgccggctccaaggttcccaagcgcccaaggcagcggagagagctagctccctcctcctcctcaggtagcgagcgagcgagctcctctgcccctctgcacacctggcctccccttctccgctgcaggaccgccgttgacgactgctccaccagtactgcgcgcgcccgcgctgcctacccttacctagccagagcgcggcgcgcgagagggagaacgaccaggaggggaggagatcgatcgatcgatcgatcgagatagatggctttcctcaacatggagcagcagacctgggccttcaccttcggtatcctaggtacatatatactgtcagtctgtcactcgtagctactaattaaccgcatcaacggagccgccctgccgtcttcgtcctcgtcagtgtttctgcattgttttccttccttgtcccaagtcctttgagacgcacgcatacatgatacatgcatggcatcatcgtctcccaagtcctaggtacagtttcatcaccgcgcgcctctctcagatatgtgtcgtcgtacaggacaacacaacacgtcctcgtcaatctactagctatatgcgctaataaccacctcttcctttccgagccacggcacagtatagcctgccatccgaacgaacggaaccgcccaaaacccaactttttttgggcctaactaatgcggtggttagcccaaacaaagaaaccagggcagtcatacattttacatgtagtacctgcttgacgacgggaaccaacacaacggcgcgccgcgaggagagtttgttccatttcctccgcctgcaggctagctgcagctaataaccccggccgccggatgttctccttctcgcatgctttttttttggggggttctttctccgcctcctgctcttcgtctgttctaccatcgtcgatcacacagcaatttgactgaccgcgtctgtccttccgaccttgccgttttgctttgcaggtaacataatctcgctgatggtgttcctttcgccgctgtaagtaactctctgctttaatttatttaacctagccgtgaacttttcatatttacgtttacagtatccagtaccatgcataatatactaccaatatgtttctatgtatattcagtaaagggaagcgtccaaaatatatactgccatcaggtatgatgagaacatatatataaagaacatggcacgcacgcatgcgtatctcactgttgaatgaatgacgtgccatcatcgtccatgctcttctagatccttttcctttaatttatgcgtggttctagatccccctatatatatatatatatatatatatatatatatatatatacgtacacctgaagacaacatagctagctaggaaataaactcgttgggaattgaattgtcgcattaataacgtcactttaatcagttgcgagccagtacgtgtcactgttcggtggttgcgttgcacatgcttttagtatggaacagcgcacgcaatctagtaataattaatctaaaaaatcacacggtaaaaaatattcgttcccgctgatcccgcgcaggccgacgttctaccgcgtgtaccgcaagaagtcgacggaggggttccagtcgaccccgtacgtggtgacgctgttcagctgcatgctgtggatcttctacgcgctgctcaagtccggcgccgagctgctggtgaccatcaacggcgtcgggtgcgtgatcgaggcggcgtacctggccgcgtacctggtgtacgcgcccaaggccgccagggcgctgacggccaagatgctgctggggctcaacgtcggcgtctttggactcgccgcgctcgccaccatggtggtctccagcgccggcctccgcgtgcgcgtgctcggctggatctgcgtcagcgtcgcgctcagcgtattcgccgcgccgctcagcatcatggcacgtatctaatttaatctctctctctctctctctctctctctctctctctctctctctgaagagggatgtgaattgttggaaccgccattaattcgctgactgccctatctacatctactttcctgcagcggcaggtggtccggaccaagagcgtggagttcatgcccatctcgctctccttcttcctggtgctgagcgccgtgatctggttcgcgtacggcgcgctgaagagggacgtgttcgtggcgttccccaacgtgctgggcttcgtcttcggcgtcgcgcagatagcgctgtacatggcgtacaggaacaaggagcccgcggcggtgacggtggaggaggcgaagctgccggagcacgccaaggaggtggtggtggccgcggcggcggccgaggccagggcgagctgcggtgctgaggtgcaccccatcgacatcgacatcgaagctacgccgacgccggtggaggaggtgcacgagccgcaggtggtcgtggtcgtcgacgtggacgtggagcctgtcacctgcgccggcgccgccgaggcagctgcgggagcaggggcagatgcgtccggcgtggccgacggcggcgtccctggacccatggcgccgccggagcagctggcgatcaagcccgacatggccatttccgtggaggcgtaggttggttaaagtgtaatagcagagtgagtcgatatcgatcagtagtagatttgtcgagtcaaggaaggggcctgctgctgctgctacgcgacagcagcggccagatgtactgcctgcatgcatgcagtccctcccgttgactcgacccgtagagaagggatgggatggaagtagccgggtccttgggcgagtagcggcgccttggcacccacgggccagccgtactggcctgtgtgatgaaccggcgagagagatgagataatgtgcgagcgagagagggttggttgggttgtggtagttgaagaagacggctagctagctgctgctgctgggctctctatctgtctctagatctgtgtatgtctgtgtaatcgaggattccctcaccttgcatgccgcctcccttttgtgtccttctagtctgatgattgttttcattccatccatgccggcgtcgcagtaataaaatttttttgg

SEQ ID NO: 30 - DNA, 1852 bp, Zea mays
ctcaggagctaatgactcacctctccgtgctcacctcctcttcttgaggcttgagctctctcttcctctctcccctcagctcatctccaccgtctccctatatataggatgctctgccggctccaaggttcccaagcgcccaaggcagcggagagagctagctccctcctcctcctcaggtagcgagcgagcgagctcctctgcccctctgcacacctggcctccccttctccgctgcaggaccgccgttgacgactgctccaccagtactgcgcgcgcccgcgctgcctacccttacctagccagagcgcggcgcgcgagagggagaacgaccaggaggggaggagatcgatcgatcgatcgatcgagatagatggctttcctcaacatggagcagcagacctgggccttcaccttcggtatcctaggtaacataatctcgctgatggtgttcctttcgccgctgccgacgttctaccgcgtgtaccgcaagaagtcgacggaggggttccagtcgaccccgtacgtggtgacgctgttcagctgcatgctgtggatcttctacgcgctgctcaagtccggcgccgagctgctggtgaccatcaacggcgtcgggtgcgtgatcgaggcggcgtacctggccgcgtacctggtgtacgcgcccaaggccgccagggcgctgacggccaagatgctgctggggctcaacgtcggcgtctttggactcgccgcgctcgccaccatggtggtctccagcgccggcctccgcgtgcgcgtgctcggctggatctgcgtcagcgtcgcgctcagcgtattcgccgcgccgctcagcatcatgcggcaggtggtccggaccaagagcgtggagttcatgcccatctcgctctccttcttcctggtgctgagcgccgtgatctggttcgcgtacggcgcgctgaagagggacgtgttcgtggcgttccccaacgtgctgggcttcgtcttcggcgtcgcgcagatagcgctgtacatggcgtacaggaacaaggagcccgcggcggtgacggtggaggaggcgaagctgccggagcacgccaaggaggtggtggtggccgcggcggcggccgaggccagggcgagctgcggtgctgaggtgcaccccatcgacatcgacatcgaagctacgccgacgccggtggaggaggtgcacgagccgcaggtggtcgtggtcgtcgacgtggacgtggagcctgtcacctgcgccggcgccgccgaggcagctgcgggagcaggggcagatgcgtccggcgtggccgacggcggcgtccctggacccatggcgccgccggagcagctggcgatcaagcccgacatggccatttccgtggaggcgtaggttggttaaagtgtaatagcagagtgagtcgatatcgatcagtagtagatttgtcgagtcaaggaaggggcctgctgctgctgctacgcgacagcagcggccagatgtactgcctgcatgcatgcagtccctcccgttgactcgacccgtagagaagggatgggatggaagtagccgggtccttgggcgagtagcggcgccttggcacccacgggccagccgtactggcctgtgtgatgaaccggcgagagagatgagataatgtgcgagcgagagagggttggttgggttgtggtagttgaagaagacggctagctagctgctgctgctgggctctctatctgtctctagatctgtgtatgtctgtgtaatcgaggattccctcaccttgcatgccgcctcccttttgtgtccttctagtctgatgattgttttcattccatccatgccggcgtcgcagtaataaaatttttttgg

SEQ ID NO: 31 - protein, 333 aa, Zea mays
MAFLNMEQQTWAFTFGILGNIISLMVFLSPLPTFYRVYRKKSTEGFQSTPYVVTLFSCMLWIFYALLKSGAELLVTINGVGCVIEAAYLAAYLVYAPKAARALTAKMLLGLNVGVFGLAALATMVVSSAGLRVRVLGWICVSVALSVFAAPLSIMRQVVRTKSVEFMPISLSFFLVLSAVIWFAYGALKRDVFVAFPNVLGFVFGVAQIALYMAYRNKEPAAVTVEEAKLPEHAKEVVVAAAAAEARASCGAEVHPIDIDIEATPTPVEEVHEPQVVVVVDVDVEPVTCAGAAEAAAGAGADASGVADGGVPGPMAPPEQLAIKPDMAISVEA

SEQ ID NO: 32 - DNA, 3120 bp, Zea mays
ccttatatagtcgactggagagttatacctgtaaatttcatcgagcttatctaaatctactttctattgctatgagtatgcctgtttttccggtgcttggttggcttctctggtttatcatccgagttatcaagtacctagccgcgtgagaaacgtgtcttcagaaatttggtaattcttatgcgatatacgtgcacggatggtatgttggtgcagtgttcttcgagatctcctcgtatccagtgctccgttgctgcatttgacttttatttcgtcaggacgaggcccatcagtacgccccaccggacgtcttattagggtcctctcccatggcccaaggagtacatttatgccttggcgacccataggatatctatctcccacaaaatatatagaactaaaaatagggtttaaggttgagctcaagaatgaatgtcctttttaaaataacattatatcgaactacttcgaatcacgactttcttgataatgtattatgttcattggagaaaatgtggctctatatacgcattgtgggcattatggtccaaaagaaaaaagagagagaaacaacaatttctatacatcaagcttgcaagtgatcgtggaaaagggaaactatatagcaaaggcacataagaatcttctatagcaaagacacacaataatctttccatgagtttaatacaccatgattatatataattataaagatagatagatatattgcaatgatataacaaagcttcggtttctaatgtgctcattgtagggttagggagtcacttctatggcgatggtgcagggtcgatgggagaagtggtggcgcgaggagggcccttgcatgtatgctcgacatgggaaatggtggtgtggggagagggctcgacgagctcaccaagaaaggtgctagaagaagagaagaagaagggcataacgaaggctgaagttatattgggcacttatcgattcttgcaaagaaccagtactaaagacctctgaccaagaactggaaaggggccattaataccagttcttacaaagaaccaatgctaaagaccttcaggatggaattgaaaaagaacctttagtaccgttcgtgtaagaaccaatactaaagagttctttagagagatggcaaactaaactacgagcgttcattagagcgatggtagacaaaacaaagcaagttacaatctctacttgaagcttattacactaagctcctctttgttcctaacgccagctcggggagcgagggttggaggttataaagcaggtacattaacactagttgaaaacaccaatcgataataatgttatgtcatcaaattccagttaaaaacaccaatcggtactaatagaattatattagtaccaattggtgtcgtcaaccgatagtgataagttaatattagttccggttagagacattaatcagtattaatgccctagctctacgaggacgcattagttaaaaccggtgctgacgaattgagtgagtaccgattttaatgcaataataggtactaatttgatttaatttactttagtgcacaacaatttgtatagtgtacaaaagttcctttaccagagacggcccttaccacagagtccaaagcaggtcggagatcgagaaccccacgcggtgcacaacaatttgtagtgtacgactctggcactgaccgatgtataattgactaaaaagcatgcacgcaaagcagctcccgcttttttcgaaaatgtagaagattatctcaatcaattaatgtaacaatttatacactctggttatatatatacatggccagaaaaaaatgtcatctattataaagctgctatatatatacatgcattgaaaaggatctttgcgtctatatataatttcacaaaaaaaagtgttaatatcatatgtgtgtgattgaagagccctgagcttgcttgtacacgatgacacaacacatgtacaccaaatcatgcgtgcatgtgccacctcgcgtgcctatataaagccacccacagccctgcctatcattgcaagagtttgagccaacacacacagagagaggacaactcctcacaactctcccttccctcctgtaggggccaaaagggttagagagtaggagaagtagttccctagcccaacacaagaaaaacaagctcgatctcctcatcaccctaatccaagcaactgctttgtgtgttgggaaattcttgtgacccctgttatattattatccgtgtagctactagcttcattcccccctttgtacccatcaaagggctggcatgtctctgcaacacccctgggcgtttgctttcggtctactaggtacatactacaccttcactaatagctaaacaagcgcccgccgcaaagctatgaattaagggcaggcatgtttggtttgctacctattttaccatactttgtctaacttttctgtctaaggttatagttcttcaattcgaacgattaatcttagccaaagtgtgacatggttagccacgaactaagcaggcccttaattattgcgcatgtatatattatatatttatctttctattctgtttaatttgttttctctttacatatatatactactatgtaagtatatattatatgcacaacaagcaggcctccttcgtgcacatatgcatagaatcaatctatacccttcatagaagccactttgagatataccttccaaaacaatcccaaaaacaagacgctcgatcttgcgctcacaatcacttagttttgcaccagattaagcatgcaccactagattttatgtactgtattacctctgccatccatggtcgatcctttagtttatcctattcatttccgtcatgaactacctgtcgagctagcaatcggtcctttatttagagtgttcagataggcatctgtctttatacaaacaataaagcctcacgaatctttagtcacaaaacaaggcaaattagacaggccacggaggtgtaaagtgtcagctctgcttatcacaacttatctctgctttatttgggcacacttttgccatacaaatggctgatcttggcgccttttttttctccttgctttgcaggtaacgtcatctccttcatgac

SEQ ID NO: 33 - DNA, 3248 bp, Zea mays
cagagctggtggagcgccatcattgcccatccggcggagctggccggatgggatgccggtcttgttctccgtgacccgagtcgattcggggtaggatgaagatggcgctccctgttgacgtggcgggtctgtgccctaggcagggtgacgtgggggctcctccgaagccgaggttgagtctgtcttctgttgccgaggccgagcccgagcaatggggtcgggcgaggcggaagtcgttcggccgaggccagggcggagtctgagccctggggtcgggcgaggcggagtttcgtcgtcttccgggtcttagccagagtccgagccctggggtcgggcggagcggagttcgccgtcttccgggtcttagcccgagtccgagccctggggtcgggcggagcggagttcgccgtcttccgggtcttagcccgagtccgagccctggggtcgggcggagcggagttcgccgtcttccgggtcttagcccgagtccgagccctggggtcgggcggagcggagttcgccgtcttccgggtcttagcccgagtccgagccctggggtcgggcggagcggagttcgccgtggcgcctttggcaaggcctgactgcctgtcagactcactctgtcgagtggcaccgcagtcggagtggcgcaggcggcgctgtccttctgtcagactggtcagaggagcggtggagtgacggcggtcacttcggctctgccgggggggcgcgtgtcaggataaaggtgtcagaccacctttgcgttaaatgcccctgcaatttggtcagtcggtgtggcgatttagtcaaggttgcttctgagcgaagccaaggcctcgggcgagccggtgatgtgtccgccgtaaaaaggggggcctcgggcgagacggaagtctctcgaggtcggctgccctcggccgaggctaggctcgggtgaagcgtgatcgagtcactcgtgtggactgatccctgacttaatcgtacccatcaggcctttgcagctttatgctgatgggggttaccagctgagaattaggcgtcttgagggaacccctaattatggtccccgacaaaaatcaacataaacatataataaattgagtcgttttaataataggaatccgtcactttatagatactgaacgtcatggataactttatcatcctaatgataatcagattctctgtaccgtcaaatttttctcaattcttaaaaaaacggttaaaaaaaaactgaccaccctacatctcttcgccgccatcgtgaggccaggccacacgcaacgcaagtgacgaccgcgcgcgtcccctgcatgcatggctcagctctcactactcccggccctgtgctgataactgtctctctttattacgtgtttacgcattttacaagatcaccttggacgaacgaacttatctcgacctgatcatatgccgggggcaatgcaagttcatttcctatgcgtcacgacaactagaccagataacgcctataactccttattggctaataaggctgtatatgatcggcacaccataagtgaatatagcagtgcaaaaaatttttttatactatatgtataaattatattgtttatagagtagagtttaaaataagaaatgagatgcaacgtaactgaccttcccattcatgtactgtatatagagcgaaggccgcctacctaaagtacgtacctgctgggatgtacacaccaacttgctgtgctgtgcaatctctcgcaaaaaaaaaaaaaaaaaaaacagtgcagtgagaggacaagggattgcgagcacaaaaggaaagcgcacgttgcatgtgccactttgtgtcatgctgatggttatctcgtttcctagctagcttagcttattagcttattcaatctccctggctgttgtcgatcaggaccaaaatggtgctgagtgtgctagctgcagggggcccggcggccggggtcactacacactatataaaccgcccctcctccaagatctctgctaccatcaagctttcaggcagggcaagcaactcgagtagtagttgccagccacacctcctgtcttcctttttccttctcgcattgtggaggagaaggatcaagtgctcataagcaagctagcttgacgaagtataggaaattaggaatacaaataaactcatcctcctcatatataaacgctcgatctccagatgtggccggcctgtccctgctgcatcccatggccttcgccttcggcctcctcggtattttttttacatttccttatttcactgtaagtgcgcttctcatcgggaaaagggtctcccctatagctaggatatacatataaaacataattttatttattcttcattattgggtcggaaggagaagtagatatatttttttttacagaaagcagggaatgagatatatatatataggacatatatgctatggtgccctgtccattatttagggcagaaagagggggaaaagaagaaaagaaaaaaggaggatatatatatatatatatttgcatgcctttgctgcctctgattaggggtggacacttttttaccataaacagaaaaccgaaccgaaatttcgatttttcgttagttcggttcggttttggtttttgtatctaagaagttcggttttcggcatcgtaatcggttttcaccgtataccgaaccgaaataccaaaaaaaccgaataccaaactttatcaattctcaaatttgactattagattatgtgaactaaatgtgtgatacaattaaattattattcacttatttgtatgtgatgtatgatgtatctctaaatatttgtgcctatataatttttatttttaaaaattatgtgtaatctatcatgtaaacttgttatatgtattgtcttaagtataagtttggtatttggtttttaccgaaaaaccaaagtaaaaaaccgaaaccgaatttctcggtttttcattttctagaaaaccgaacggtttctaatgtacgaagaaccgaagttttataaaaccgaaaaccgaaccgaagtttagaaaaaaaccgaatgcccagccctgatggtgtggcgtgacatcacaaagcgtcgtcgtcatcgaaattgattgaaggagatagttgcattgcatgtgtcatgctcatgcacagagcacgagcaccccagctgaattctatctgacgcctctcgctcctttttcttttgtttggcggtttgctgtttgtgtttgcaggcaacatcatctcc

SEQ ID NO: 34 - DNA, 2373 bp, Zea mays
ataaaaaatgttttaacagttgtttatttgaactcgtagttgtaatgtaatctttttttaaaaaaaaaagacttaatcaagttagagaagcatgatttaatataaaactaaaatttagaagagcggcctgctcatagagttttgtttagttaatacataaacatcattctcttgtatgctatgcatataaagaatatatgtattctaaaacatatataccaggatcggaacaagcacacgcgcgtacgtcgtagcttctgcatggctctagtaagctccagcgcgcagcgcgacgacgtctcttgctgtaatgattgatacacatgctttgtcagtccgtcgttaccagcaaatcatttgtaatgatacactcgctcgctcgctcgcgtagtcgcagcaaattaaaactggctagcctgcagctagctgatatgcgactgggaggagtataattagcgccaccgcatttgtttgcgtacgtttctggctccggtggacgatccatgcatgattggaagcggtggatcgatgcgcacgacacgtacggcagctagcggggatcatagcgtccgcgggagggagaagccagtgccagcccaagctggcatggctggctcgcatcgcgcatatatacgcaaacgtggcaggtttcagcttgggcaccgtaccgctagcctaggctgccgctggagctggacgacagagccgattgcaagaagccaggatcagaattagatggaccctaggcatgcagtttgattgagcttggacatccatcatatatcatcgttggaagctataagctagctagctagctagcatcacgtacgtgctgctacgtacatttggctctcgatcgatcagaggccggccggtgtcgtcgtcgtcgtagactcgtaggtatgaaaataattgtacggcggtcagatcatttccgtctcgatcgatcgtccttgggttgtccgtttttagtttttactactacgtactgtacatgcgagcgtttaccttaaaagcatcgaaacgctcgtcgttagcctgctgcgtcaagtgcagtgagtacctccactccgatccaggcggctgcatcaagcagcaaatgcaagctagaagggatgccccaagcacactagctagctagtactagtccacaaatacgcagctcacaccttgccatatatcccggtcatatcatacgtgtaccacccacccttaccgtatccgtgtatgcgggcgggggtgggtcacccattaattgccggcgatattttggcggtaacgtggcgccgcgaaccaggctagcagggagccaggggctaggcggctagcaccacagacgatctggagcagagcacatgacatgcgacctcgaggagcgccccccaccctgaaccccccacgggacggaacacgtaaagagcacttccattccattccgtccattccaccagcaggaagaaggaacgaccacatcactgcttcccgttcgttcgttcggcacgcagctgcctgcgcctgcgtgtgtgtgtgcacgacttgcttgcgaggccagcactggtgtgcagtccgttggccctccccccgcaaagttcgtcacgacgctgggatgtgccgtgtcttttgtagaaagcgtgagcgggccgttacgacaactagcagggcacacaagggcaagtgcaggcgaggcgactcacgtacgtacagttagcgtgtgaccagctagctgctgctgcgcctgcgccgcatcatcagacgccctgcaggaaccgcggggcttgggccggacgccgccgccgccgcctctgcctctgctggtggtggagtttgacatgtcacgaggcgttacgacgaaacgaaaacagagccactgagccagtgcgacgcgcaaccacaaaatagatggccggctaccctctctctctttctctccccccgcggcgccaaaaaagcaagcgcgacgcgacaagtcgttgtgtaatcatgtagatctaacgggtccactcaggagctaatgactcacctctccgtgctcacctcctcttcttgaggcttgagctctctcttcctctctcccctcagctcatctccaccgtctccctatatataggatgctctgccggctccaaggttcccaagcgcccaaggcagcggagagagctagctccctcctcctcctcaggtagcgagcgagcgagctcctctgcccctctgcacacctggcctccccttctccgctgcaggaccgccgttgacgactgctccaccagtactgcgcgcgcccgcgctgcctacccttacctagccagagcgcggcgcgcgagagggagaacgaccaggaggggaggagatcgatcgatcgatcgatcgagatag

(19) United States
(12) Patent Application Publication
(10) Pub. No.: US 2017/0275641 A1
(43) Pub. Date: Sep. 28, 2017

(54) COMPOSITIONS AND METHODS FOR IMPROVING ABIOTIC STRESS TOLERANCE

(71) Applicant: Syngenta Participations AG, Basel (CH)

(72) Inventor: Shib Sankar BASU, Apex, NC (US)

(73) Assignee: Syngenta Participations AG, Basel (CH)

(21) Appl. No.: 15/502,690

(22) PCT Filed: Dec. 17, 2015
(86) PCT No.: PCT/US15/66298

§ 371 Date: Feb. 8, 2017

Related U.S. Application Data
(60) Provisional application No. 62/093,044, filed on Dec. 17, 2014.

(51) Int. Cl. C12N 15/82; C12Q 1/68; A01H 1/02; C07K 14/415

(52) U.S. Cl. CPC C12N 15/8261; C12N 15/8201; C12N 15/8273; C12N 15/8271; C07K 14/415; C12Q 1/6895; A01H 1/02

(57) ABSTRACT

The present invention relates to compositions and methods for improving yield, yield stability, and/or drought stress tolerance in plants. Plants and/or plant parts identified, selected and/or produced using compositions and methods of the present invention are also provided.

### RELATED APPLICATION INFORMATION

This Application claims the benefit of U.S. Provisional Application No. 62/093,044, filed on 17 Dec. 2014, the contents of which are incorporated herein by reference.

### STATEMENT REGARDING ELECTRONIC FILING OF A SEQUENCE LISTING

A Sequence Listing in ASCII text format, submitted under 37 C.F.R. §1.821, entitled 80605-WO-REG-ORG-P-1_Sequence_Listing_ST25, 82 kilobytes in size, generated on Dec. 16, 2015 and filed via EFS-Web, is provided in lieu of a paper copy. This Sequence Listing is hereby incorporated by reference into the specification for its disclosures.

### FIELD OF THE INVENTION

The present invention relates to compositions and methods for improving yield, yield stability, and/or drought stress tolerance in plants.

### BACKGROUND OF THE INVENTION

To keep pace with future food productivity requirements, increasing the yield and/or yield stability of a plant may be desirable. Abiotic stress is a major factor affecting the normal growth and development of plants and limiting crop yields. At present, the impact of drought stress on crop yields around the world ranks first among abiotic stress factors; the damage caused by drought is equivalent to the damage caused by all natural disasters combined and has become the predominant obstruction to agricultural development in many areas.

Identifying genes that enhance yield, yield stability, and/or the drought tolerance of a plant could lead to more efficient crop production by allowing for the identification, selection and production of plants with enhanced yield, yield stability, and/or drought stress tolerance.

### SUMMARY OF THE INVENTION

The present invention provides abiotic stress tolerant plants and/or plant parts, as well as methods and compositions for identifying, selecting and/or producing abiotic stress tolerant plants and/or plant parts. Some embodiments provide drought stress tolerant plants and/or plant parts, as well as methods and compositions for identifying, selecting and/or producing drought stress tolerant plants and/or plant parts. In some embodiments, plants and/or plant parts having increased yield and/or increased yield stability are provided, as well as methods and compositions for identifying, selecting and/or producing plants and/or plant parts having increased yield and/or increased yield stability.

In some embodiments, the present invention provides a method of increasing yield, increasing yield stability, and/or enhancing drought stress tolerance in a plant and/or plant part, the method comprising expressing, in the plant and/or plant part, an exogenous nucleic acid comprising one or more of the nucleotide sequences set forth in any one of SEQ ID NOs: 1 to 11, 29 to 30, 32 to 34, one or more nucleotide sequences that encodes a polypeptide comprising the amino acid sequence of any one of SEQ ID NOs: 12 to 16, 31, one or more nucleotide sequences that is at least 70%, 75%, 80%, 85%, 90%, 91%, 92%, 93%, 94%, 95%, 96%, 97%, 98%, 99%, 99.5% or more identical to the nucleotide sequence set forth in any one of SEQ ID NOs: 1 to 11, 29 to 30, 32 to 34, one or more nucleotide sequences that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence of any one of SEQ ID NOs: 12 to 16, 31, one or more nucleotide sequences that is complementary to one of the aforementioned nucleotide sequences, one or more nucleotide sequences that specifically hybridize to one of the aforementioned nucleotide sequences under stringent hybridization conditions, and/or a functional fragment of one of the aforementioned nucleotide sequences. In some embodiments, the method further comprises introducing the exogenous nucleic acid into the plant and/or plant part. In some embodiments, the exogenous nucleic acid is operably linked to a promoter which is a tissue-specific promoter and/or a drought inducible promoter. Examples of such promoters include the MADS promoter, the OsMADS promoters, OsMADS6 promoters, OsMADS7 promoters, SWEET13 promoters, SWEET14 promoters or SWEET15 promoters.

In some embodiments, the present invention provides a nonnaturally occurring or exogenous nucleic acid that encodes one or more sugar (e.g., sucrose) transporters and/or one or more proteins capable of increasing the expression, stability and/or activity of one or more sugar (e.g., sucrose) transporters and/or capable of decreasing the expression and/or concentration of trehalose-6-phosphate (T6P) in a plant and/or plant part. In some embodiments, the present invention provides a nonnaturally occurring or exogenous nucleic acid comprising a nucleic acid capable of driving transcription in a plant selected from the group of SEQ ID NOs: 32, 33, or 34, a nucleic acid that is at least 70% identical to a nucleic acid selected from the group of SEQ ID NOs: 32, 33, or 34, one or more nucleotide sequences that specifically hybridize to one of the aforementioned nucleotide sequences under stringent hybridization conditions, and/or a functional fragment of one or more of the aforementioned nucleotide sequences.

In some embodiments, the present invention provides an expression cassette, vector, transgenic bacterium, virus, fungal cell, plant and/or plant part that comprises a nonnaturally occurring or exogenous nucleic acid comprising one or more of the nucleotide sequences set forth in any one of SEQ ID NOs: 1 to 11, 29 to 30, 32 to 34, one or more of the nucleotide sequences that encode a polypeptide comprising the amino acid sequence of any one of SEQ ID NOs: 12 to 16, 31 one or more nucleotide sequences that is at least 70% identical to the nucleotide sequence set forth in any one of SEQ ID NOs: 1 to 11, 29 to 30, 32 to 34, one or more nucleotide sequences that encode a polypeptide comprising an amino acid sequence that is at least 70% identical to the amino acid sequence of any one of SEQ ID NOs: 12 to 16, 31 one or more nucleotide sequences that is complementary to one of the aforementioned nucleotide sequences, one or more nucleotide sequences that specifically hybridize to any one of the aforementioned nucleotide sequences under stringent hybridization conditions, and/or a functional fragment of one or more of the aforementioned nucleotide sequences further comprises a trehalose-6-phosphate phosphatase. In some embodiments, the trehalose-6-phosphate phosphatase comprises one or more of the nucleotide sequences set forth in SEQ ID NOs: 17 to 20 and/or one or more of the nucleotide sequences that encode a polypeptide comprising the amino acid sequence of any one of SEQ ID NOs: 21 to 24.

The foregoing and other objects and aspects of the present invention are explained in detail in the specification set forth below.

### DETAILED DESCRIPTION

The present invention provides compositions and methods for identifying, selecting and/or producing plants and/or plant parts having enhanced abiotic stress tolerance, as well as plants and/or plant parts identified, selected and/or produced using compositions and methods of the present invention. Some embodiments provide compositions and methods for identifying, selecting and/or producing plants and/or plant parts having increased yield, increased yield stability, and/or enhanced drought stress tolerant, as well as plants and/or plant parts identified, selected and/or produced using compositions and methods of the present invention.

Although the following terms are believed to be well understood by one of ordinary skill in the art, the following definitions are set forth to facilitate understanding of the presently disclosed subject matter.

All technical and scientific terms used herein, unless otherwise defined below, are intended to have the same meaning as commonly understood by one of ordinary skill in the art.

References to techniques employed herein are intended to refer to the techniques as commonly understood in the art, including variations on those techniques or substitutions of equivalent techniques that would be apparent to one of skill in the art.

All patents, patent publications, non-patent publications referenced herein are incorporated by reference in their entireties for the teachings relevant to the sentence or paragraph in which the reference is presented. In case of a conflict in terminology, the present specification is controlling.

As used herein, the terms “a” or “an” or “the” may refer to one or more than one, unless the context clearly and unequivocally indicates otherwise. For example, “an” endogenous nucleic acid can mean one endogenous nucleic acid or a plurality of endogenous nucleic acids.

As used herein, the term “and/or” refers to and encompasses any and all possible combinations of one or more of the associated listed items, as well as the lack of combinations when interpreted in the alternative (“or”).

As used herein, the term “about,” when used in reference to a measurable value such as an amount of mass, dose, time, temperature, and the like, refers to a variation of ±0.1%, 0.25%, 0.5%, 0.75%, 1%, 2%, 3%, 4%, 5%, 6%, 7%, 8%, 9%, 10%, 15% or even 20% of the specified value as well as the specified value. Thus, if a given composition is described as comprising “about 50% X,” it is to be understood that, in some embodiments, the composition comprises 50% X whilst in other embodiments it may comprise anywhere from 40% to 60% X (i.e., 50%±10%).

As used herein, the terms “abiotic stress” and “abiotic stress conditions” refer to non-living factors that negatively affect a plant's ability to grow, reproduce and/or survive (e.g., drought, flooding, extreme temperatures, extreme light conditions, extreme osmotic pressures, extreme salt concentrations, high winds, natural disasters and poor edaphic conditions (e.g., extreme soil pH, nutrient-deficient soil, compacted soil, etc.)).

As used herein, the terms “abiotic stress tolerance” and “abiotic stress tolerant” refer to a plant's ability to endure and/or thrive under abiotic stress conditions (e.g., drought stress conditions, osmotic stress conditions, salt stress conditions and/or temperature stress conditions). When used in reference to a plant part, the terms refer to the ability of a plant that arises from that plant part to endure and/or thrive under abiotic stress conditions.

As used herein, the terms “backcross” and “backcrossing” refer to the process whereby a progeny plant is repeatedly crossed back to one of its parents. In a backcrossing scheme, the “donor” parent refers to the parental plant with the desired allele or locus to be introgressed. The “recipient” parent (used one or more times) or “recurrent” parent (used two or more times) refers to the parental plant into which the gene or locus is being introgressed. The initial cross gives rise to the F1 generation. The term “BC1” refers to the second use of the recurrent parent, “BC2” refers to the third use of the recurrent parent, and so on.

As used herein, the transitional phrase “consisting essentially of” is to be interpreted as encompassing the recited materials or steps and those that do not materially affect the basic and novel characteristic(s) of the claimed invention.

As used herein, the terms “cross,” “crossing” and “crossed” refer to the fusion of gametes to produce progeny (e.g., cells, seeds or plants). The term encompasses both sexual crosses (e.g., the pollination of one plant by another or the combination of protoplasts from two distinct plants via protoplast fusion) and selfing (e.g., self-pollination wherein the pollen and ovule are from the same plant).

As used herein, the terms “cultivar” and “variety” refer to a group of similar plants that by structural or genetic features and/or performance can be distinguished from other cultivars/varieties within the same species.

As used herein, the terms “decrease,” “decreases,” “decreasing” and similar terms refer to a reduction of at least about 5%, 10%, 15%, 20%, 25%, 30%, 35%, 40%, 45%, 50%, 55%, 60%, 65%, 70%, 75%, 80%, 85%, 90%, 95%, 96%, 97%, 98%, 99%, 99.5% or more. In some embodiments, the reduction results in no or essentially no activity (i.e., an insignificant or undetectable amount of activity).

As used herein, the term “enhanced abiotic stress tolerance” and grammatical variations thereof refers to an improvement in the ability of a plant and/or plant part to grow, reproduce and/or survive under abiotic stress conditions, as compared to one or more controls (e.g., a native plant/plant part of the same species). “Enhanced” may refer to any improvement in a plant's or plant part's ability to thrive and/or endure when grown under stress conditions, including, but not limited to, enhanced drought stress tolerance, osmotic stress tolerance, salt stress tolerance and/or temperature stress tolerance. In some embodiments, enhanced abiotic stress tolerance is evidenced by decreased water loss, decreased accumulation of one or more reactive oxygen species, decreased accumulation of one or more salts, increased salt excretion, increased accumulation of one or more dehydrins, improved root architecture, improved osmotic pressure regulation, increased accumulation of one or more late embryogenesis abundant proteins, increased survival rate, increased growth rate, increased height, increased chlorophyll content, increased sugar concentration and/or availability, increased yield stability, and/or increased yield (e.g., increased biomass, increased seed yield, increased grain sugar content (GSC), increased grain yield at standard moisture percentage (YGSMN), increased grain moisture at harvest (GMSTP), increased grain weight per plot (GWTPN), increased percent yield recovery (PYREC), decreased yield reduction (YRED), and/or decreased percent barren (PB)) when grown under abiotic stress conditions. A plant or plant part that exhibits enhanced abiotic stress tolerance may be designated as “abiotic stress tolerant.”

As used herein, the term “enhanced drought tolerance” refers to an improvement in one or more water optimization traits and/or drought stress tolerant phenotypes as compared to one or more controls (e.g., a native plant/plant part of the same species). A plant or plant part that exhibits decreased water loss, decreased accumulation of one or more reactive oxygen species, decreased accumulation of one or more salts, increased salt excretion, increased accumulation of one or more dehydrins, improved root architecture, improved osmotic pressure regulation, increased accumulation of one or more late embryogenesis abundant proteins, increased survival rate, increased growth rate, increased height, increased chlorophyll content, increased sugar concentration and/or availability, increased yield stability, and/or increased yield (e.g., increased biomass, increased seed yield, increased GSC, increased YGSMN, increased GMSTP, increased GWTPN, increased PYREC, decreased YRED, and/or decreased PB) as compared to a control plant (e.g., one or both of its parents) when each is grown under the same or substantially the same drought stress conditions displays enhanced drought tolerance and may be designated as “drought tolerant.”

In some embodiments, the plant and/or plant part exhibits an increased survival rate after being subjected to polyethylene glycol (PEG)-simulated drought stress conditions (e.g., incubation in a 200 g/L PEG6000 solution). In some embodiments, the plant and/or plant part exhibits an increased yield (e.g., increased seed yield and/or biomass) after being subjected to PEG-simulated drought stress conditions (e.g., incubation in a 200 g/L PEG6000 solution). In some embodiments, the plant and/or plant part exhibits an increased carbon (e.g., sugar, such as, sucrose) concentration and/or availability after being subjected to PEG-simulated drought stress conditions (e.g., incubation in a 200 g/L PEG6000 solution). The increased carbon concentration and/or availability in the plant and/or plant part may be in a particular plant tissue, such as, for example, a reproductive and/or sink tissue (e.g., a flowering tissue and/or seed). In some embodiments, the increased carbon concentration and/or availability in the plant and/or plant part may be present in a particular plant tissue that is developing (e.g., the increased carbon concentration and/or availability may be present in a plant tissue during the growth and/or developmental stage of the tissue).

In some embodiments, the plant and/or plant part exhibits an increased survival rate after being subjected to a managed stress environment (MSE) in which water supply is controlled to impose a water deficit during a given time interval for the plant and/or plant part (e.g., 1, 2, 3, 4, or more weeks). The MSE may maintain the plant and/or plant part under water deficit conditions (e.g., may maintain the water level at a given value or within a given range) prior to, during, and/or after a particular stage of growth and development of the plant and/or plant part. In some embodiments, the MSE may maintain the plant and/or plant part under water deficit conditions prior to, during, and/or after the flowering period of the plant and/or plant part. For example, the MSE may maintain the plant and/or plant part under water deficit conditions throughout the entire flowering period or during 1, 2, 3, 4, or more weeks of the flowering period of the plant and/or plant part. In some embodiments, the plant and/or plant part exhibits an increased yield (e.g., increased seed yield and/or biomass) after being subjected to a MSE in which water supply is controlled to impose a water deficit during a given time interval for the plant and/or plant part (e.g., during part or all of the flowering period). In some embodiments, the plant and/or plant part exhibits an increased carbon (e.g., sugar, such as, sucrose) concentration and/or availability after being subjected to a MSE in which water supply is controlled to impose a water deficit during a given time interval for the plant and/or plant part (e.g., during part or all of the flowering period). The increased carbon concentration and/or availability in the plant and/or plant part may be in a particular plant tissue, such as, for example, a reproductive and/or sink tissue (e.g., a flowering tissue and/or seed). In some embodiments, the increased carbon concentration and/or availability in the plant and/or plant part may be present in a particular plant tissue that is developing (e.g., the increased carbon concentration and/or availability may be present in a plant tissue during the growth and/or developmental stage of the tissue).

As used herein, the term “water optimization trait” refers to any trait that can be shown to influence the growth and/or development of a plant under different sets of growth conditions related to water availability (e.g., drought stress conditions).

It is to be understood that a “drought tolerant” and/or “drought stress tolerant” plant and/or plant part may also be referred to as an “abiotic stress tolerant” plant and/or plant part because drought stress is an abiotic stress.

As used herein, the term “expression cassette” refers to a nucleic acid capable of directing expression of a particular nucleotide sequence in a host cell. The expression cassette may be chimeric, meaning that at least one of its components is heterologous with respect to at least one of its other components. The expression cassette may also be one that is naturally occurring but has been obtained in a recombinant form useful for heterologous expression. Typically, the expression cassette is heterologous with respect to the host (i.e., one or more of the nucleic acid sequences in the expression cassette do(es) not occur naturally in the host cell and must have been introduced into the host cell or an ancestor of the host cell by a transformation event).

As used herein, with respect to nucleic acids, the term “exogenous” refers to a nucleic acid that is not in the natural genetic background of the cell/organism in which it resides. Thus, an exogenous nucleic acid may also be referred to as a nonnaturally occurring nucleic acid. In some embodiments, the exogenous nucleic acid comprises one or more nucleic acid sequences that are not found in the natural genetic background of the cell/organism. In some embodiments, the exogenous nucleic acid comprises one or more additional copies of a nucleic acid that is endogenous to the cell/organism.

As used herein with respect to nucleotide sequences, the terms “express” and “expression” refer to transcription and/or translation of the sequences.

As used herein with respect to nucleic acids, the term “fragment” refers to a nucleic acid that is reduced in length relative to a reference nucleic acid and that comprises, consists essentially of and/or consists of a nucleotide sequence of contiguous nucleotides identical or almost identical (e.g., 90%, 91%, 92%, 93%, 94%, 95%, 96%, 97%, 98%, 99% identical) to a corresponding portion of the reference nucleic acid. Such a nucleic acid fragment may be, where appropriate, included in a larger polynucleotide of which it is a constituent. In some embodiments, the nucleic acid fragment comprises, consists essentially of or consists of at least about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450, 500, or more consecutive nucleotides. In some embodiments, the nucleic acid fragment comprises, consists essentially of or consists of less than about 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, 350, 400, 450 or 500 consecutive nucleotides.

As used herein with respect to polypeptides, the term “fragment” refers to a polypeptide that is reduced in length relative to a reference polypeptide and that comprises, consists essentially of and/or consists of an amino acid sequence of contiguous amino acids identical or almost identical (e.g., 90%, 91%, 92%, 93%, 94%, 95%, 96%, 97%, 98%, 99% identical) to a corresponding portion of the reference polypeptide. Such a polypeptide fragment may be, where appropriate, included in a larger polypeptide of which it is a constituent. In some embodiments, the polypeptide fragment comprises, consists essentially of or consists of at least about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, 300, or more consecutive amino acids. In some embodiments, the polypeptide fragment comprises, consists essentially of or consists of less than about 2, 3, 4, 5, 6, 7, 8, 9, 10, 11, 12, 13, 14, 15, 20, 25, 30, 35, 40, 45, 50, 55, 60, 65, 70, 75, 80, 85, 90, 95, 100, 125, 150, 175, 200, 225, 250, or 300 consecutive amino acids.

As used herein with respect to nucleic acids, the term “functional fragment” refers to nucleic acid that encodes a functional fragment of a polypeptide.

As used herein with respect to polypeptides, the term “functional fragment” refers to polypeptide fragment that retains at least about 20%, 25%, 30%, 35%, 40%, 45%, 50%, 55%, 60%, 65%, 70%, 75%, 80%, 85%, 90%, 95%, 96%, 97%, 98%, 99%, 99.5% or more of at least one biological activity of the full-length polypeptide (e.g., enzymatic activity). In some embodiments, the functional fragment actually has a higher level of at least one biological activity of the full-length polypeptide.

As used herein, the term “germplasm” refers to genetic material of or from an individual plant, a group of plants (e.g., a plant line, variety or family), or a clone derived from a plant line, variety, species, or culture. The genetic material can be part of a cell, tissue or organism, or can be isolated from a cell, tissue or organism.

As used herein, the term “heterologous” refers to a nucleotide/polypeptide that originates from a foreign species, or, if from the same species, is substantially modified from its native form in composition and/or genomic locus by deliberate human intervention.

As used herein, the terms “increase,” “increases,” “increasing” and similar terms refer to an elevation of at least about 20%, 25%, 30%, 35%, 40%, 45%, 50%, 75%, 100%, 125%, 150%, 175%, 200%, 350%, 300%, 350%, 400%, 450%, 500% or more.

As used herein, the term “informative fragment” refers to a nucleotide sequence comprising a fragment of a larger nucleotide sequence, wherein the fragment allows for the identification of one or more alleles within the larger nucleotide sequence. For example, an informative fragment of the nucleotide sequence of SEQ ID NO:1 comprises a fragment of the nucleotide sequence of SEQ ID NO:1 and allows for the identification of one or more alleles located within the portion of the nucleotide sequence corresponding to that fragment of SEQ ID NO:1.

As used herein with respect to nucleic acids, polynucleotides and polypeptides, the term “isolated” refers to a nucleic acid, polynucleotide or polypeptide that, by the hand of man, exists apart from its native environment and is therefore not a product of nature. In some embodiments, the nucleic acid, polynucleotide or polypeptide exists in a purified form that is substantially free of cellular material, viral material, culture medium (when produced by recombinant DNA techniques), or chemical precursors or other chemicals (when chemically synthesized). An “isolated fragment” is a fragment of a polynucleotide or polypeptide that is not naturally occurring as a fragment and would not be found in the natural state. “Isolated” does not mean that the preparation is technically pure (homogeneous), but rather that it is sufficiently pure to provide the polynucleotide or polypeptide in a form in which it can be used for the intended purpose. In certain embodiments, the composition comprising the polynucleotide or polypeptide is at least about 50%, 55%, 60%, 65%, 70%, 75%, 80%, 85%, 90%, 95%, 96%, 97%, 98%, or 99% or more pure.

As used herein with respect to cells, the term “isolated” refers to a cell that, by the hand of man, exists apart from its native environment and is therefore not a product of nature. In some embodiments, the cell is separated from other components with which it is normally associated in its natural state. For example, an isolated plant cell may be a plant cell in culture medium and/or a plant cell in a suitable carrier. “Isolated” does not mean that the preparation is technically pure (homogeneous), but rather that it is sufficiently pure to provide the cell in a form in which it can be used for the intended purpose. In certain embodiments, the composition comprising the cell is at least about 50%, 55%, 60%, 65%, 70%, 75%, 80%, 85%, 90%, 95%, 96%, 97%, 98%, or 99% or more pure.

As used herein with respect to nucleic acids, the term “nonfunctional fragment” refers to nucleic acid that encodes a nonfunctional fragment of a polypeptide.

As used herein with respect to polypeptides, the term “nonfunctional fragment” refers to polypeptide fragment that exhibits none or essentially none (i.e., less than about 10%, 9%, 8%, 7%, 6%, 5%, 4%, 3%, 2%, 1% or less) of the biological activities of the full-length polypeptide. As used herein with respect to nucleic acids, proteins, plants, plant parts, bacteria, viruses and fungi, the term “nonnaturally occurring” refers to nucleic acids, proteins, plants, plant parts, bacteria, viruses or fungi that do not naturally exist in nature. In some embodiments, a nonnaturally occurring nucleic acid does not naturally exist in nature in that it is not in the natural genetic background of the cell/organism in which it resides. Thus, a plant, plant part, bacteria, virus and/or fungi of the present invention comprising the nonnaturally occurring nucleic acid may also be nonnaturally occurring and/or may express a nonnaturally occurring protein. In some embodiments, a nonnaturally occurring nucleic acid, protein, plant, plant part, bacteria, virus, and/or fungi of the present invention may comprise any suitable variation(s) from their closest naturally occurring counterparts. For example, nonnaturally occurring or exogenous nucleic acids of the present invention may comprise an otherwise naturally occurring nucleotide sequence having one or more point mutations, insertions or deletions relative to the naturally occurring nucleotide sequence, the nucleic acid could be modified through codon optimized to improve expression, a copy of the otherwise naturally occurring nucleic acid is introduced into a new chromosomal position or locus, or the introns of the naturally occurring nucleic acid have been removed to create a cDNA nucleic acid. In some embodiments, nonnaturally occurring nucleic acids of the present invention comprise a naturally occurring nucleotide sequence and one or more heterologous nucleotide sequences (e.g., one or more heterologous promoter sequences, intron sequences and/or termination sequences). Likewise, nonnaturally occurring proteins of the present invention may comprise an otherwise naturally occurring protein that comprises one or more mutations, insertions, additions or deletions relative to the naturally occurring protein (e.g., one or more epitope tags). Similarly, nonnaturally occurring plants, plant parts, bacteria, viruses and fungi of the present invention may comprise one more exogenous nucleotide sequences and/or one or more nonnaturally occurring copies of a naturally occurring nucleotide sequence (i.e., extraneous copies of a gene that naturally occurs in that species). Nonnaturally occurring plants and plant parts may be produced by any suitable method, including, but not limited to, transforming/transfecting/transducing a plant or plant part with an exogenous nucleic acid and crossing a naturally occurring plant or plant part with a nonnaturally occurring plant or plant part. It is to be understood that all nucleic acids, proteins, plants, plant parts, bacteria, viruses and fungi claimed herein are nonnaturally occurring.

Also as used herein, the terms “nucleic acid,” “nucleic acid molecule,” “nucleotide sequence” and “polynucleotide” can be used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. The term “nucleic acid,” unless otherwise limited, encompasses analogues having the essential nature of natural nucleotide sequences in that they hybridize to single-stranded nucleic acids in a manner similar to naturally occurring nucleotides (e.g., peptide nucleic acids).

Where single-stranded, the nucleic acid can be a sense strand or an antisense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention.

Nucleic acid molecules and/or nucleotide sequences provided herein are presented herein in the 5′ to 3′ direction, from left to right and are represented using the standard code for representing the nucleotide characters as set forth in the U.S. sequence rules, 37 CFR §§1.821-1.825 and the World Intellectual Property Organization (WIPO) Standard ST.25.

Different nucleic acids or proteins having homology are referred to herein as “homologues.” The term homologue includes homologous sequences from the same and other species and orthologous sequences from the same and other species.

As used herein, the term “nucleotide” refers to a monomeric unit from which DNA or RNA polymers are constructed and which consists of a purine or pyrimidine base, a pentose, and a phosphoric acid group. Nucleotides (usually found in their 5′-monophosphate form) are referred to by their single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

The term “homology” in the context of the invention refers to the level of similarity between nucleic acid or amino acid sequences in terms of nucleotide or amino acid identity or similarity, respectively, i.e., sequence similarity or identity. Homology, homologue, and homologous also refers to the concept of similar functional properties among different nucleic acids or proteins. Homologues include genes that are orthologous and paralogous. Homologues can be determined by using the coding sequence for a gene, disclosed herein or found in appropriate database (such as that at NCBI or others) in one or more of the following ways. For an amino acid sequence, the sequences should be compared using algorithms (for instance see section on “identity” and “substantial identity”). For nucleotide sequences the sequence of one DNA molecule can be compared to the sequence of a known or putative homologue in much the same way. Homologues are at least 20% identical, or at least 30% identical, or at least 40% identical, or at least 50% identical, or at least 60% identical, or at least 70% identical, or at least 80% identical, or at least 88% identical, or at least 90% identical, or at least 92% identical, or at least 95% identical, across any substantial region of the molecule (DNA, RNA, or protein molecule).

In some embodiments, a homologue of this invention can have a substantial sequence similarity or identity (e.g., 70%, 75%, 80%, 81%, 82%, 83%, 84%, 85%, 86%, 87%, 88%, 89%, 90%, 91%, 92%, 93%, 94%, 95%, 96%, 97%, 98%, 99%, and/or 100%) to the nucleotide or polypeptide sequences of the invention.

“Identity” or “percent identity” refers to the degree of similarity between two nucleic acid or amino acid sequences. For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

“Identity” can be readily calculated by known methods including, but not limited to, those described in: Computational Molecular Biology (Lesk, A. M., ed.) Oxford University Press, New York (1988); Biocomputing: Informatics and Genome Projects (Smith, D. W., ed.) Academic Press, New York (1993); Computer Analysis of Sequence Data, Part I (Griffin, A. M., and Griffin, H. G., eds.) Humana Press, New Jersey (1994); Sequence Analysis in Molecular Biology (von Heinje, G., ed.) Academic Press (1987); and Sequence Analysis Primer (Gribskov, M. and Devereux, J., eds.) Stockton Press, New York (1991).

As used herein, the term “percent sequence identity” or “percent identity” refers to the percentage of identical nucleotides in a linear polynucleotide sequence of a reference (“query”) polynucleotide molecule (or its complementary strand) as compared to a test (“subject”) polynucleotide molecule (or its complementary strand) when the two sequences are optimally aligned. In some embodiments, “percent identity” can refer to the percentage of identical amino acids in an amino acid sequence.

Sequence comparison between two or more polynucleotides is generally performed by comparing portions of the two sequences over a comparison window to identify and compare local regions of sequence similarity. The “percentage of sequence identity” for polynucleotides, such as about 50, 55, 60, 65, 70, 75, 80, 85, 90, 95, 98, 99 or 100 percent sequence identity, can be determined by comparing two optimally aligned sequences over a comparison window, wherein the portion of the polynucleotide sequence in the comparison window can include additions or deletions (i.e., gaps) as compared to the reference sequence for optimal alignment of the two sequences. The percentage is calculated by: (a) determining the number of positions at which the identical nucleic acid base occurs in both sequences; (b) dividing the number of matched positions by the total number of positions in the window of comparison; and (c) multiplying the result by 100.

Optimal alignment of sequences for comparison can be conducted, e.g., by the local homology algorithm of Smith & Waterman, Adv. Appl. Math. 2: 482 (1981), by the homology alignment algorithm of Needleman & Wunsch, J. Mol. Biol. 48: 443 (1970), by the search for similarity method of Pearson & Lipman, Proc. Nat'l. Acad. Sci. USA 85: 2444 (1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally, Ausubel et al., infra).

One example of an algorithm that is suitable for determining percent sequence identity and sequence similarity is the BLAST algorithm, which is described in Altschul et al., J. Mol. Biol. 215: 403-410 (1990). Software for performing BLAST analyses is publicly available through the National Center for Biotechnology Information (http://www.ncbi.nlm nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., 1990). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always>0) and N (penalty score for mismatching residues; always<0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached. The BLAST algorithm parameters W, T, and X determine the sensitivity and speed of the alignment.

The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix (see Henikoff & Henikoff, Proc. Natl. Acad. Sci. USA 89: 10915 (1989)).

In addition to calculating percent sequence identity, the BLAST algorithm also performs a statistical analysis of the similarity between two sequences (see, e.g., Karlin & Altschul, Proc. Nat'l. Acad. Sci. USA 90: 5873-5787 (1993)). One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

Another widely used and accepted computer program for performing sequence alignments is CLUSTALW v1.6 (Thompson, et al. Nuc. Acids Res., 22: 4673-4680, 1994). The number of matching bases or amino acids is divided by the total number of bases or amino acids, and multiplied by 100 to obtain a percent identity. For example, if two 580 base pair sequences had 145 matched bases, they would be 25 percent identical. If the two compared sequences are of different lengths, the number of matches is divided by the shorter of the two lengths. For example, if there were 100 matched amino acids between a 200 and a 400 amino acid proteins, they are 50 percent identical with respect to the shorter sequence. If the shorter sequence is less than 150 bases or 50 amino acids in length, the number of matches are divided by 150 (for nucleic acid bases) or 50 (for amino acids), and multiplied by 100 to obtain a percent identity.

The phrase “substantially identical,” in the context of two nucleic acids or two amino acid sequences, refers to two or more sequences or subsequences that have at least about 50% nucleotide or amino acid residue identity when compared and aligned for maximum correspondence as measured using one of the following sequence comparison algorithms or by visual inspection. In certain embodiments, substantially identical sequences have at least about 60%, or at least about 70%, or at least about 80%, or even at least about 90% or 95% nucleotide or amino acid residue identity. In certain embodiments, substantial identity exists over a region of the sequences that is at least about 50 residues in length, or over a region of at least about 100 residues, or the sequences are substantially identical over at least about 150 residues. In further embodiments, the sequences are substantially identical when they are identical over the entire length of the coding regions.

Thus, in some embodiments of the invention, the substantial identity exists over a region of the sequences that is at least about 50, about 60, about 70, about 80, about 90, about 100, about 110, about 120, about 130, about 140, about 150, or more residues in length. In some particular embodiments, the sequences are substantially identical over at least about 150 residues. In representative embodiments, substantially identical nucleotide or protein sequences perform substantially the same function (e.g., conferring increased drought tolerance).

For sequence comparison, typically one sequence acts as a reference sequence to which test sequences are compared. When using a sequence comparison algorithm, test and reference sequences are entered into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

An “identity fraction” for aligned segments of a test sequence and a reference sequence is the number of identical components which are shared by the two aligned sequences divided by the total number of components in the reference sequence segment, i.e., the entire reference sequence or a smaller defined part of the reference sequence.

Two nucleotide sequences can also be considered to be substantially complementary when the two sequences hybridize to each other under stringent conditions. In some representative embodiments, two nucleotide sequences considered to be substantially complementary hybridize to each other under highly stringent conditions.

As used herein with respect to nucleic acids, the term “operably linked” refers to a functional linkage between two or more nucleic acids. For example, a promoter sequence may be described as being “operably linked” to a heterologous nucleic acid sequence because the promoter sequences initiates and/or mediates transcription of the heterologous nucleic acid sequence. In some embodiments, the operably linked nucleic acid sequences are contiguous and/or are in the same reading frame. In some embodiments, the operably linked nucleic acid sequences are not contiguous.

As used herein, the term “sugar transporter” refers to a protein that transports one or more sugars in a cell. A sugar transporter may import sugar into a cell and/or into an organelle within a cell and/or may export sugar from a cell and/or from an organelle within a cell. In some embodiments, a sugar transporter may transport a sugar, such as, for example, a monosaccharide (e.g., pentose, glucose, mannose, fructose, etc.), a disaccharide (e.g., sucrose, maltose, etc.), and/or an oligosaccharide. In some embodiments, a sugar transporter may be a sucrose transporter (i.e., a protein that transports sucrose). A sugar transporter, such as, for example, a sucrose transporter, may traverse a cell and/or organelle membrane one or more times, such as, but not limited to, 2, 3, 4, 5, 6, 7, 8, 9, or more times. In some embodiments, a sucrose transporter may traverse a cell and/or organelle membrane 5, 6, or 7 times. In some embodiments, a sugar transporter may form a pore. The pore may be formed by one or more transmembrane domains of the transporter and/or the sub-domains thereof, such as, for example, by a spherical arrangement of the one or more transmembrane domains of the transporter and/or the sub-domains thereof. In some embodiments, the pore may allow for the passage of a sugar through it. The pore may be selective for the passage of a sugar only. In some embodiments, the pore may have one or more selective point(s) that restrict the passage to certain sized or certain shaped molecules. In some embodiments, passage through the pore may be based on a concentration gradient. In some embodiments, the pore may be opened and/or closed based on the activity of a cofactor, such as, for example, the activity of an interacting protein, the binding of an ion, and/or the presence of a charge, such as a negative or positive charge. Example sugar transporters include, but are not limited to, those described in U.S. Patent Application Publication No. 2011/0209248 and International Publication No. WO 2013/086494, the contents of each of which are incorporated herein by reference in their entirety.

In some embodiments, the sugar transporter may be a sucrose transporter, such as a SWEET protein. Example SWEET proteins include, but are not limited to, SWEET 13 proteins (e.g., a SWEET 13a, SWEET 13b, SWEET 13c, and/or SWEET 13cδ protein), SWEET 14 proteins (e.g., a SWEET 14a and/or SWEET 14b) and SWEET 15 (e.g, SWEET 15a and SWEET 15b). SWEET 13, SWEET 14 and SWEET 15 are considered CLAD III sugar transporters and can be identified through a highly conserved domain as described in International Publication No. WO 2013/086494. V-M/F-Y/V-A-G-S/A-S/P/L-S-M/X/I-V-A/M-I-L-V/X/X/V/I-V/K-X/T-S/K-R-E/S/V-A/E-K-Q-A/Y-F/M/P/F/X/L-M/S (SEQ ID NO:25). The conserved domain may be between the fifth and sixth transmembrane domains of a seven transmembrane transporter. SWEET transporters from various species have been identified, for example, Arabidospsis thaliana, rice, corn, Citrus sinensi, Medicago trunculate, wheat, soybean, petunia, poplar, grape, barley, sorghum, spruce, lotus, tabocco and tomato.

In some embodiments, a SWEET 13 protein has an amino acid sequence that is at least about 70%, 75%, 80%, 85%, 90%, 91%, 92%, 93%, 94%, 95%, 96%, 97%, 98%, 99%, 99.5% or more identical to the amino acid sequence of any one of SEQ ID NOs: 12 or 14 to 16 and/or to a functional fragment thereof. In some embodiments, a SWEET 13 protein has an amino acid sequence that is substantially identical to the amino acid sequence of any one of SEQ ID NOs: 12 or 14 to 16 and/or to a functional fragment thereof. In some embodiments, a SWEET 13 protein comprises 1, 2, 3, 4, 5, 6, or 7 alpha-helical transmembrane domains.

In some embodiments, a SWEET 14 protein has an amino acid sequence that is at least about 70%, 75%, 80%, 85%, 90%, 91%, 92%, 93%, 94%, 95%, 96%, 97%, 98%, 99%, 99.5% or more identical to the amino acid sequence of SEQ ID NO:13 and/or to a functional fragment thereof. In some embodiments, a SWEET 14 protein has an amino acid sequence that is substantially identical to the amino acid sequence of SEQ ID NO:13 and/or a functional fragment thereof. In some embodiments, the SWEET 14 protein comprises 1, 2, 3, 4, 5, 6, or 7 alpha-helical transmembrane domains.

In some embodiments, a SWEET 15 protein has an amino acid sequence that is at least about 70%, 75%, 80%, 85%, 90%, 91%, 92%, 93%, 94%, 95%, 96%, 97%, 98%, 99%, 99.5% or more identical to the amino acid sequence of SEQ ID NO: 31 and/or to a functional fragment thereof. In some embodiments, a SWEET 15 protein has an amino acid sequence that is substantially identical to the amino acid sequence of SEQ ID NO: 31 and/or to a functional fragment thereof. In some embodiments, a SWEET 15 protein comprises 1, 2, 3, 4, 5, 6, or 7 alpha-helical transmembrane domains.

In some embodiments, a SWEET 13 protein, a SWEET 14 protein and/or SWEET 15 protein may comprise a domain having the sequence: V-M/F-Y/V-A-G-S/A-S/P/L-S-M/X/l-V-A/M-1-L-V/X/X/V/1-V/K-X/T-S/K-R-E/S/V-A/E-K-Q-A/Y-F/M/P/F/X/L-M/S (SEQ ID NO:25). In some embodiments, this domain may be between the fifth and sixth transmembrane domains of a SWEET 13 protein, a SWEET 14 protein and/or a SWEET 15 protein. In some embodiments, a SWEET 13 protein, a SWEET 14 protein and/or a SWEET 15 protein may comprise one or more of the following sequences: K-R-A/K-N-S/K/S-T/T-S-I-A/E-K-Q-G/G-S-C/F-Y/Q-S-E-H/S-A/I-L-V-T/P/Y/X/V-S-T-C/A-S-T/L/F-L-A/S/A-C-S-T/M-T-G-L/L/W-F-L/I-L-M-V/Y-F-L/Y/A-G/X/K-R-Q-S-T (SEQ ID NO:26), optionally in the second transmembrane domain or between the second and third transmembrane domains of a SWEET 13 protein, a SWEET 14 protein and/or a SWEET 15 protein; V-M/F/V-A/A-S/P/L/S-A-F-M-T/I-V/I-M-V/X/X/V/I-V-M/K-R-Q/T-S/K-R/S/V/E-A/Y-F/M-L/P/F-I/X/L/S (SEQ ID NO:27), optionally between the fifth and sixth transmembrane domains of a SWEET 13 protein, a SWEET 14 protein and/or a SWEET 15 protein; and/or P/N/V-I-G-T/L-G-V-I/G/F-L-A/X/F-L/G-S/X/X/Q/M/X/X/Y-F/X/X/Y-F (SEQ ID NO:28), optionally in the seventh transmembrane domain of a SWEET 13 protein, a SWEET 14 protein and/or a SWEET 15 protein.

As used herein, the term “T6PP protein” refers to a trehalose 6-phosphate phosphatase (T6PP) protein. Example T6PP proteins include, but are not limited to, those described in U.S. Patent Application Publication No. 2013/0019342, U.S. Patent Application Publication No. 2014/0143908, and International Publication No. WO 2005/102034, the contents of each of which are incorporated herein by reference in their entirety. In some embodiments, a nucleic acid that encodes a T6PP protein has a nucleotide sequence that is at least about 70%, 75%, 80%, 85%, 90%, 91%, 92%, 93%, 94%, 95%, 96%, 97%, 98%, 99%, 99.5% or more identical to the nucleotide sequence of any one of SEQ ID NOs: 17 to 20 and/or to a functional fragment thereof. In some embodiments, a nucleic acid that encodes a T6PP protein has a nucleotide sequence that is substantially identical to the nucleotide sequence of any one of SEQ ID NOs: 17 to 20 and/or a functional fragment thereof. In some embodiments, a T6PP protein has an amino acid sequence that is at least about 70%, 75%, 80%, 85%, 90%, 91%, 92%, 93%, 94%, 95%, 96%, 97%, 98%, 99%, 99.5% or more identical to the amino acid sequence of any one of SEQ ID NOs: 21 to 24 and/or to a functional fragment thereof. In some embodiments, the T6PP protein has an amino acid sequence that is substantially identical to the amino acid sequence of any one of SEQ ID NOs: 21 to 24 and/or a functional fragment thereof.

As used herein, the term “percent barren” (PB) refers to the percentage of plants in a given area (e.g., plot) with no grain. It is typically expressed in terms of the percentage of plants per plot and can be calculated as:

$$\frac{number of plants in the plot with no grain}{total number of plants in the plot} \times 100$$

As used herein, the term “percent yield recovery” (PYREC) refers to the effect a nucleotide sequence and/or combination of nucleotide sequences has on the yield of a plant grown under stress conditions (e.g., drought stress conditions) as compared to that of a control plant that is genetically identical except insofar as it lacks the nucleotide sequence and/or combination of nucleotide sequences. PYREC is calculated as:

$$1 - \frac{ \begin{matrix}\begin{matrix}\begin{matrix}yield under non \text{-} stress \\ ( w \text{/} nucleotide sequence ( s ) of interest ) -\end{matrix} \\ yield under stress conditions\end{matrix} \\ ( w \text{/} nucleotide sequence ( s ) of interest )\end{matrix}}{\begin{matrix}\begin{matrix}\begin{matrix}yield under non \text{-} stress \\ ( w \text{/} out nucleotide sequence ( s ) of interest ) -\end{matrix} \\ yield under stress conditions\end{matrix} \\ ( w \text{/} out nucleotide sequence ( s ) of interest )\end{matrix}} \times 100$$

By way of example and not limitation, if a control plant yields 200 bushels under full irrigation conditions, but yields only 100 bushels under drought stress conditions, then its percentage yield loss would be calculated at 50%. If an otherwise genetically identical hybrid that contains the nucleotide sequence(s) of interest yields 125 bushels under drought stress conditions and 200 bushels under full irrigation conditions, then the percentage yield loss would be calculated as 37.5% and the PYREC would be calculated as 25% [1.00−(200−125)/(200−100)×100)].

As used herein, the terms “phenotype,” “phenotypic trait” or “trait” refer to one or more traits of an organism. The phenotype can be observable to the naked eye, or by any other means of evaluation known in the art, e.g., microscopy, biochemical analysis, or an electromechanical assay. In some cases, a phenotype is directly controlled by a single gene or genetic locus, i.e., a “single gene trait.” In other cases, a phenotype is the result of several genes. It is noted that, as used herein, the term “water optimization phenotype” takes into account environmental conditions that might affect water optimization such that the water optimization effect is real and reproducible.

As used herein, the term “plant cell” refers to a cell existing in, taken from and/or derived from a plant (e.g., a cell derived from a plant cell/tissue culture). Thus, the term “plant cell” may refer to an isolated plant cell, a plant cell in a culture, a plant cell in an isolated tissue/organ and/or a plant cell in a whole plant.

As used herein, the term “plant part” refers to at least a fragment of a whole plant or to a cell culture or tissue culture derived from a plant. Thus, the term “plant part” may refer to a plant cell, a plant tissue and/or a plant organ, as well as to a cell/tissue culture derived from a plant cell, plant tissue or plant culture. Embodiments of the present invention may comprise and/or make use of any suitable plant part, including, but not limited to, anthers, branches, buds, calli, clumps, cobs, cotyledons, ears, embryos, filaments, flowers, fruits, husks, kernels, leaves, lodicules, ovaries, palea, panicles, pedicels, pods, pollen, protoplasts, roots, root tips, seeds, silks, stalks, stems, stigma, styles, and tassels. In some embodiments, the plant part is a plant germplasm.

As used herein, the term “polynucleotide” refers to a deoxyribopolynucleotide, ribopolynucleotide or analogs thereof that have the essential nature of a natural deoxyribopolynucleotide/ribonucleotide in that they hybridize, under stringent hybridization conditions, to substantially the same nucleotide sequence as naturally occurring nucleotides and/or allow translation into the same amino acid(s) as the naturally occurring nucleotide(s). A polynucleotide can be full-length or a subsequence of a native or heterologous structural or regulatory gene. Unless otherwise indicated, the term includes reference to the specified sequence as well as the complementary sequence thereof. Thus, DNAs or RNAs with backbones modified for stability or for other reasons are “polynucleotides” as that term is intended herein. Moreover, DNAs or RNAs comprising unusual bases, such as inosine or modified bases, such as tritylated bases, to name just two examples, are polynucleotides as the term is used herein. It will be appreciated that a great variety of modifications have been made to DNA and RNA that serve many useful purposes known to those of skill in the art. The term polynucleotide as it is employed herein embraces such chemically, enzymatically or metabolically modified forms of polynucleotides, as well as the chemical forms of DNA and RNA characteristic of viruses and cells, including inter alia, simple and complex cells.

As used herein, the terms “polypeptide,” “peptide” and “protein” refer to a polymer of amino acid residues. The terms encompass amino acid polymers in which one or more amino acid residue is an artificial chemical analogue of a corresponding naturally occurring amino acid, as well as to naturally occurring amino acid polymers.

As used herein, the terms “progeny” and “progeny plant” refer to a plant generated from a vegetative or sexual reproduction from one or more parent plants. A progeny plant may be obtained by cloning or selfing a single parent plant, or by crossing two parental plants.

As used herein, the terms “promoter” and “promoter sequence” refer to nucleic acid sequences involved in the regulation of transcription initiation. A “plant promoter” is a promoter capable of initiating transcription in plant cells. Exemplary plant promoters include, but are not limited to, those that are obtained from plants, from plant viruses and from bacteria that comprise genes expressed in plant cells such Agrobacterium or Rhizobium. A “tissue-specific promoter” is a promoter that preferentially initiates transcription in a certain tissue (or combination of tissues). A “stress-inducible promoter” is a promoter that preferentially initiates transcription under certain environmental conditions (or combination of environmental conditions). A “developmental stage-specific promoter” is a promoter that preferentially initiates transcription during certain developmental stages (or combination of developmental stages).

As used herein, the term “regulatory sequences” refers to nucleotide sequences located upstream (5′ non-coding sequences), within or downstream (3′ non-coding sequences) of a coding sequence, which influence the transcription, RNA processing or stability, or translation of the associated coding sequence. Regulatory sequences include, but are not limited to, promoters, enhancers, exons, introns, translation leader sequences, termination signals, and polyadenylation signal sequences. Regulatory sequences include natural and synthetic sequences as well as sequences that can be a combination of synthetic and natural sequences. An “enhancer” is a nucleotide sequence that can stimulate promoter activity and can be an innate element of the promoter or a heterologous element inserted to enhance the level or tissue specificity of a promoter. The coding sequence can be present on either strand of a double-stranded DNA molecule, and is capable of functioning even when placed either upstream or downstream from the promoter.

Where a clone comprising a promoter has been isolated in accordance with the instant invention, one may wish to delimit the essential promoter regions within the clone. One efficient, targeted means for preparing mutagenized promoters relies upon the identification of putative regulatory elements within the promoter sequence. This can be initiated by comparison with promoter sequences known to be expressed in similar tissue specific or developmentally unique patterns. Sequences which are shared among promoters with similar expression patterns are likely candidates for the binding of transcription factors and are thus likely elements which confer expression patterns. Confirmation of these putative regulatory elements can be achieved by deletion analysis of each putative regulatory sequence followed by functional analysis of each deletion construct by assay of a reporter gene which is functionally attached to each construct. As such, once a starting promoter sequence is provided, any of a number of different deletion mutants of the starting promoter could be readily prepared.

Functional fragments of SWEET 13, 14 or 15 promoters or regulatory sequence may be 50, 100, 150, 200, 250, 300, 350, 400, 450, 500, 550, 600, 650, 700, 750, 800, 850, 900, 950, 1000 or more base pairs. Narrowing the transcription regulating nucleic acid to its essential, transcription mediating elements can be realized in vitro by trial-and-error deletion mutations, or in silico using promoter element search routines. Regions essential for promoter activity often demonstrate clusters of certain, known promoter elements. Such analysis can be performed using available computer algorithms such as PLACE (“Plant Cis-acting Regulatory DNA Elements”; Higo Nucl. Acids Res. 27 (1): 297-300 (1999), the BIOBASE database “Transfac” Wingender Nucl. Acids Res. 29 (1): 281-283 (2001) or the database PlantCARE Lescot Nucl. Acids Res. 30 (1): 325-327 (2002).

For example, functional borders, genetic fine structure, and distance requirements of cis elements mediating light responsiveness of the parsley chalcone synthase promoter Proc Natl Acad Sci USA 87:5387-5391(1990); Terzaghi W B, Cashmore A R Light-regulated transcription Annu Rev Plant Physiol Plant Mol Biol 46:445-474 (1995); Nakashima K, Fujita Y, Katsura K, Maruyama K, Narusaka Y, Seki M, Shinozaki K, Yamaguchi-Shinozaki K. Transcriptional regulation of ABI3- and ABA-responsive genes including RD29B and RD29A in seeds, germinating embryos, and seedlings of Arabidopsis. Plant Mol Biol. 60: 51-68 (2006); Piechulla B, Merforth N, Rudolph B Identification of tomato Lhc promoter regions necessary for circadian expression Plant Mol Biol 38:655-662 (1998); Villain P, Mache R, Zhou D X The mechanism of GT element-mediated cell type-specific transcriptional control J Biol Chem 271:32593-32598 (1996); Le Gourrierec J, Li Y F, Zhou D X Transcriptional activation by Arabidopsis GT-1 may be through interaction with TFIIA-TBP-TATA complex Plant J 18:663-668 (1999); Buchel A S, Brederode F T, Bol J F, Linthorst H J M Mutation of GT-1 binding sites in the Pr-1A promoter influences the level of inducible gene expression in vivo Plant Mol Biol 40:387-396 (1999); Zhou D X Regulatory mechanism of plant gene transcription by GT-elements and GT-factors Trends in Plant Science 4:210-214 (1999); Giuliano G, Pichersky E, Malik V S, Timko M P, Scolnik P A, Cashmore A R An evolutionarily conserved protein binding sequence upstream of a plant light regulated gene. Proc Natl Acad Sci USA 85:7089-7093 (1988); Donald R G K, Cashmore A R Mutation of either G box or 1 box sequences profoundly affects expression from the Arabidopsis rbcS-1A promoter. EMBO J 9:1717-1726 (1990); Rose A, Meier I, Wienand U The tomato I-box binding factor LeMYBI is a member of a novel class of Myb-like proteins Plant J 20: 641-652 (1999); Martinez-Hernandez A, Lopez-Ochoa L, Arguello-Astorga, G, Herrera-Estrella L. Functional properties and regulatory complexity of a minimal RBCS light-responsive unit activated by phytochrome, cryptochrome, and plastid signals. Plant Physiol. 128:1223-1233 (2002); Nakamura M, Tsunoda T, Obokata J Photosynthesis nuclear genes generally lack TATA-boxes: a tobacco photosystem I gene responds to light through an initiator Plant J 29: 1-10 (2002); Castresana C, Garcia-Luque I, Alonso E, Malik V S, Cashmore A R Both positive and negative regulatory elements mediate expression of a photoregulated CAB gene from Nicotiana plumbaginifolia EMBO J 7:1929-1936 (1988); Hudson M E, Quail P H. Identification of promoter motifs involved in the network of phytochrome A-regulated gene expression by combined analysis of genomic sequence and microarray data. Plant Physiol. 133: 1605-1616 (2003); Jiao Y, Ma L, Strickland E, Deng X W. Conservation and Divergence of Light-Regulated Genome Expression Patterns during Seedling Development in Rice and Arabidopsis. Plant Cell. 17: 3239-3256 (2005)).

Promoter activity can be routinely confirmed by expression assays, for example, as described in the Examples section herewith. In addition, modification of promoter sequences without loss of activity is routine in the art. For example, the well-known CaMV 35S promoter has been shown to retain promoter activity when fragmented into two domains, with Domain A (−90 to +8) able to confer expression primarily in root tissues (Benfey et. al., (1989) EMBO J 8(8):2195-2202 and Domain B (−343 to −90) conferring expression in most cell types of leaf, stem and root vascular tissues. A CaMV promoter has been truncated to a −46 promoter and still retains, although reduced, correct promoter activity (Odell et. al., (1985) Nature 313:810-812).

Welsch et. al. describe the creation of multiple deletion fragments of an Arabidopsis thaliana phytoene synthase gene promoter (Welsch et. al. (2003) Planta 216:523-534). Using truncation studies, Welsch et. al. showed that as little as 11% of the promoter needed to be retained in order to observe some promoter activity. The deletion analysis of promoters from the cab 1A, cab 1B, cab 8 and cab 11 genes from the tomato light harvesting complex of genes determined which deletion would affect circadian expression (Piechulla, et. al. (1998) Plant Molecular Biology 38:655-662). A deletion of approximately 775 bp could be made from a 1058 bp plant promoter designated AtEXP18 without significantly reducing promoter activity (Cho and Cosgrove (2002) Plant Cell 14:3237-3253). In addition, the authors showed that numerous substitution mutations could be made in a fragment of AtEXP18, while retaining full promoter activity and in some cases increasing activity.

The invention disclosed herein provides polynucleotide molecules comprising regulatory element/promoter fragments that may be used in constructing novel chimeric regulatory elements. Novel combinations comprising fragments of these polynucleotide molecules and at least one other regulatory element or fragment can be constructed and tested in plants and are considered to be within the scope of this invention. Thus the design, construction, and use of chimeric regulatory elements may be one embodiment of this invention. Promoters of the present invention include homologues of cis elements known to effect gene regulation that show homology with the promoter sequences of the present invention. These cis elements include but are not limited to light regulatory elements.

Functional equivalent fragments of one of the transcription regulating nucleic acids described herein comprise at least 50, 100, 150, 200, 250, 300, 350, 400, 450, 500, 550, 600, 650, 700, 750, 800, 850, 900, 950, or 1000 base pairs of a transcription regulating nucleic acid as described by SEQ ID NOS. 1 to 15. Equivalent fragments of transcription regulating nucleic acids, which are obtained by deleting the region encoding the 5′-untranslated region of the mRNA, would then only provide the (untranscribed) promoter region. The 5′-untranslated region can be easily determined by methods known in the art (such as 5′-RACE analysis). Accordingly, some of the transcriptions regulating nucleic acids, as described herein, are equivalent fragments of other sequences.

As indicated above, deletion mutants of the promoter of the invention also could be randomly prepared and then assayed. Following this strategy, a series of constructs are prepared, each containing a different portion of the promoter (a subclone), and these constructs are then screened for activity. A suitable means for screening for activity is to attach a deleted promoter or intron construct which contains a deleted segment to a selectable or screenable marker, and to isolate only those cells expressing the marker gene. In this way, a number of different, deleted promoter constructs are identified which still retain the desired, or even enhanced, activity. The smallest segment which is required for activity is thereby identified through comparison of the selected constructs. This segment may then be used for the construction of vectors for the expression of exogenous genes. [00130] Furthermore, it is contemplated that promoters combining elements from more than one promoter may be useful. For example, U.S. Pat. No. 5,491,288 discloses combining a Cauliflower Mosaic Virus promoter with a histone promoter. Thus, the elements from the promoters disclosed herein may be combined with elements from other promoters. Promoters which are useful for plant transgene expression include those that are inducible, viral, synthetic, constitutive (Odell Nature 313: 810-812 (1985)), temporally regulated, spatially regulated, tissue specific, and spatial temporally regulated. Using the regulatory elements described herein, numerous agronomic genes can be expressed in transformed plants. More particularly, plants can be genetically engineered to express various phenotypes of agronomic interest. Exemplary genes implicated in this regard include, but are not limited to, those categorized below.

1. Pests or Disease Resistance Nucleic Acids, for Example:

(A) Plant disease resistance nucleic acids. Plant defenses are often activated by specific interaction between the product of a disease resistance gene (R) in the plant and the product of a corresponding avirulence (Avr) gene in the pathogen. A plant can be transformed with a cloned resistance gene to engineer plants that are resistant to specific pathogen strains. See, for example Jones et al., Science 266: 789 (1994) (cloning of the tomato Cf-9 gene for resistance to Cladosporium fulvum); Martin et al., Science 262: 1432 (1993) (tomato Pto gene for resistance to Pseudomonas syringae pv. tomato encodes a protein kinase); Mindrinos et al., Cell 78: 1089 (1994) (Arabidopsis RSP2 gene for resistance to Pseudomonas syringae). A developmental-arrestive protein produced in nature by a pathogen or a parasite. Thus, fungal endo .alpha.-1,4-D-polygalacturonases facilitate fungal colonization and plant nutrient release by solubilizing plant cell wall homo-.alpha.-1,4-D-galacturonase. See Lamb et al., Bio/Technology 10: 1436 (1992). The cloning and characterization of a gene which encodes a bean endopolygalacturonase-inhibiting protein is described by Toubart et al., Plant J. 2: 367 (1992). A molecule that stimulates signal transduction. For example, see the disclosure by Botella et al., Plant Molec. Biol. 24: 757 (1994), of nucleotide sequences for mung bean calmodulin cDNA clones, and Griess et al., Plant Physiol. 104: 1467 (1994), who provide the nucleotide sequence of a maize calmodulin cDNA clone. A hydrophobic moment peptide. See PCT application WO95/16776 (disclosure of peptide derivatives of Tachyplesin which inhibit fungal plant pathogens) and PCT application WO95/18855 (teaches synthetic antimicrobial peptides that confer disease resistance), the respective contents of which are hereby incorporated by reference. A membrane permease, a channel former or a channel blocker. For example, see the disclosure by Jaynes et al., Plant Sci. 89: 43 (1993), of heterologous expression of a cecropin-.beta. lytic peptide analog to render transgenic tobacco plants resistant to Pseudomonas solanacearum. A viral-invasive protein or a complex toxin derived therefrom. For example, the accumulation of viral coat proteins in transformed plant cells imparts resistance to viral infection and/or disease development effected by the virus from which the coat protein gene is derived, as well as by related viruses. See Beachy et al., Ann. Rev. Phytopathol. 28: 451 (1990). Coat protein-mediated resistance has been conferred upon transformed plants against alfalfa mosaic virus, cucumber mosaic virus, tobacco streak virus, potato virus X, potato virus Y, tobacco etch virus, tobacco rattle virus and tobacco mosaic virus. Id. An insect-specific antibody or an immunotoxin derived therefrom. Thus, an antibody targeted to a critical metabolic function in the insect gut would inactivate an affected enzyme, killing the insect. Cf. Taylor et al., Abstract #497, Seventh Int'l Symposium on Molecular Plant-Microbe Interactions (Edinburgh, Scotland, 1994) (enzymatic inactivation in transgenic tobacco via production of single-chain antibody fragments). A virus-specific antibody. See, for example, Tavladoraki et al., Nature 366: 469 (1993), who show that transgenic plants expressing recombinant antibody genes are protected from virus attack. A developmental-arrestive protein produced in nature by a plant. For example, Logemann et al., Bio/Technology 10: 305 (1992), have shown that transgenic plants expressing the barley ribosome-inactivating gene have an increased resistance to fungal disease.

(B) Pest Resistance Nucleic Acids. A Bacillus thuringiensis protein, a derivative thereof or a synthetic polypeptide modeled thereon. See, for example, Geiser et al., Gene 48: 109 (1986), who disclose the cloning and nucleotide sequence of a Bt .delta.-endotoxin gene. Moreover, DNA molecules encoding .delta.-endotoxin genes can be purchased from American Type Culture Collection (Rockville, Md.), for example, under ATCC Accession Nos. 40098, 67136, 31995 and 31998. A lectin. See, for example, the disclosure by Van Damme et al., Plant Molec. Biol. 24: 25 (1994), who disclose the nucleotide sequences of several Clivia miniata mannose-binding lectin genes. A vitamin-binding protein, such as avidin. See PCT application US93/06487 the contents of which are hereby incorporated by. The application teaches the use of avidin and avidin homologues as larvicides against insect pests. An enzyme inhibitor, for example, a protease inhibitor or an amylase inhibitor. See, for example, Abe et al., J. Biol. Chem. 262: 16793 (1987) (nucleotide sequence of rice cysteine proteinase inhibitor), Huub et al., Plant Molec. Biol. 21: 985 (1993) (nucleotide sequence of cDNA encoding tobacco proteinase inhibitor I), and Sumitani et al., Biosci. Biotech. Biochem. 57: 1243 (1993) (nucleotide sequence of Streptomyces nitrosporeus .alpha.-amylase inhibitor). An insect-specific hormone or pheromone such as an ecdysteroid and juvenile hormone, a variant thereof, a mimetic based thereon, or an antagonist or agonist thereof. See, for example, the disclosure by Hammock et al., Nature 344: 458 (1990), of baculovirus expression of cloned juvenile hormone esterase, an inactivator of juvenile hormone. An insect-specific peptide or neuropeptide which, upon expression, disrupts the physiology of the affected pest. For example, see the disclosures of Regan, J. Biol. Chem. 269: 9 (1994) (expression cloning yields DNA coding for insect diuretic hormone receptor), and Pratt et al., Biochem. Biophys. Res. Comm 163: 1243 (1989) (an allostatin is identified in Diploptera puntata). See also U.S. Pat. No. 5,266,317 to Tomalski et al., who disclose genes encoding insect-specific, paralytic neurotoxins. Insect-specific venom produced in nature by a snake, a wasp, etc. For example, see Pang et al., Gene 116: 165 (1992), for disclosure of heterologous expression in plants of a gene coding for a scorpion insect toxic peptide. An enzyme responsible for a hyperaccumulation of a monterpene, a sesquiterpene, a steroid, hydroxamic acid, a phenylpropanoid derivative or another non-protein molecule with insecticidal activity. An enzyme involved in the modification, including the post-translational modification, of a biologically active molecule; for example, a glycolytic enzyme, a proteolytic enzyme, a lipolytic enzyme, a nuclease, a cyclase, a transaminase, an esterase, a hydrolase, a phosphatase, a kinase, a phosphorylase, a polymerase, an elastase, a chitinase and a glucanase, whether natural or synthetic. See PCT application WO 93/02197 in the name of Scott et al., which discloses the nucleotide sequence of a callase gene. DNA molecules which contain chitinase-encoding sequences can be obtained, for example, from the ATCC under Accession Nos. 39637 and 67152. See also Kramer et al., Insect Biochem. Molec. Biol. 23: 691 (1993), who teach the nucleotide sequence of a cDNA encoding tobacco hookworm chitinase, and Kawalleck et al., Plant Mole. Biol. 21: 673 (1993), who provide the nucleotide sequence of the parsley ubi4-2 polyubiquitin gene.

2. Herbicide Resistance Nucleic Acids, for Example:

An herbicide that inhibits the growing point or meristem, such as an imidazalinone or a sulfonylurea. Exemplary genes in this category code for mutant ALS and AHAS enzyme as described, for example, by Lee et al., EMBO J. 7: 1241 (1988), and Miki et al., Theor Appl. Genet. 80: 449 (1990), respectively. Glyphosate (resistance imparted by mutant 5-enolpyruvl-3-phosphikimate synthase (EPSP) and aroA genes, respectively) and other phosphono compounds such as glufosinate (phosphinothricin acetyl transferase (PAT) and Streptomyces hygroscopicus phosphinothricin acetyl transferase (bar) genes), and pyridinoxy or phenoxy proprionic acids and cycloshexones (ACCase inhibitor-encoding genes). See, for example, U.S. Pat. No. 4,940,835 to Shah et al., which discloses the nucleotide sequence of a form of EPSP which can confer glyphosate resistance. A DNA molecule encoding a mutant aroA gene can be obtained under ATCC accession No. 39256, and the nucleotide sequence of the mutant gene is disclosed in U.S. Pat. No. 4,769,061 to Comai. European patent application No. 0 333 033 and U.S. Pat. No. 4,975,374 describe nucleotide sequences of glutamine synthetase genes which confer resistance to herbicides such as L-phosphinothricin. The nucleotide sequence of a phosphinothricin-acetyl-transferase gene is provided in European application No. 0 242 246; De Greef et al., Bio/Technology 7: 61 (1989), describe the production of transgenic plants that express chimeric bar genes coding for phosphinothricin acetyl transferase activity. Exemplary of genes conferring resistance to phenoxy proprionic acids and cycloshexones, such as sethoxydim and haloxyfop, are the Acc1-S1, Acc1-S2 and Acc1-S3 genes described by Marshall et al., Theor. Appl. Genet. 83: 435 (1992). An herbicide that inhibits photosynthesis, such as a triazine (psbA and gs+ genes) and a benzonitrile (nitrilase gene). Przibilla et al., Plant Cell 3: 169 (1991), describe the transformation of Chlamydomonas with plasmids encoding mutant psbA genes. Nucleotide sequences for nitrilase genes are disclosed in U.S. Pat. No. 4,810,648 to Stalker, and DNA molecules containing these genes are available under ATCC Accession Nos. 53435, 67441 and 67442. Cloning and expression of DNA coding for a glutathione S-transferase is described by Hayes et al., Biochem. J. 285: 173 (1992).

3. Value-Added Trait Nucleic Acids, for Example:

Modified fatty acid metabolism, for example, by transforming a plant with an antisense gene of stearoyl-ACP desaturase to increase stearic acid content of the plant. See Knultzon et al., Proc. Natl. Acad. Sci. USA 89: 2624 (1992). Introduction of a phytase-encoding gene would enhance breakdown of phytate, adding more free phosphate to the transformed plant. For example, see Van Hartingsveldt et al., Gene 127: 87 (1993), for a disclosure of the nucleotide sequence of an Aspergillus niger phytase gene. Modified carbohydrate composition effected, for example, by transforming plants with a gene coding for an enzyme that alters the branching pattern of starch. See Shiroza et al., J. Bacteriol. 170: 810 (1988) (nucleotide sequence of Streptococcus mutans fructosyltransferase gene), Steinmetz et al., Mol. Gen. Genet. 200: 220 (1985) (nucleotide sequence of Bacillus subtilis levansucrase gene), Pen et al., Bio/Technology 10: 292 (1992) (production of transgenic plants that express Bacillus licheniformis .alpha.-amylase), Elliot et al., Plant Molec. Biol. 21: 515 (1993) (nucleotide sequences of tomato invertase genes) and Fisher et al., Plant Physiol. 102: 1045 (1993) (maize endosperm starch branching enzyme II).

4. Photoassimilation Regulation Nucleic Acids, for Example:

Any of the enzymes or genes involved in the C3, C4 or CAM photosynthesis/photorespiration pathway may be operably linked to any of the regulatory nucleic acids described herein. Enzymes may include rubisco (ribulose bisphosphate carboxylase/oxygenase, EC 4.1.1.39), phosphoglycollate phosphatase (EC 3.1.3.18), (S)-2-hydroxy-acid oxidase (EC 1.1.3.15), glycine transaminase (EC 2.6.1.4), serine-glyoxylate aminotransferase (EC 2.6.1.45), glycerate dehydrogenase (EC 1.1.1.29), glycerate kinase (2.7.1.31); phosphoenolpyruvate carboxylase (PEPC, EC 4.1.1.31), NADP-dependent malic enzyme (NADPMD) or malate dehydrogenase (EC 1.1.1.40, EC 1.1.1.82), phosphoglycerate kinase (PGK, EC 2.7.2.3), sedoheptulose-1,7-bisphosphatase (SBP, EC 3.1.3.37), fructose-1, 6-bisphosphate phosphatase (FBPase, EC 3.1.3.11), phosphoribulokinase (PRK, EC 2.7.1.19), pyruvate, orthophosphate dikinase (PPDK, EC 2.7.9.1), and the like. Numerous examples of the photoassimilation regulation genes can be found in the literature. The BRENDA database (brenda.enzymes.org) can be queried for sequence information on many of the genes involved in the photosynthesis/photorespiration pathways. In particular, examples of PRK, SBP, PGK and NADPME from maize can be found in WO2012061585, which is hereby incorporated by reference. Typical C3 plants include wheat, rice, soybean and potato. Typical C4 plants are primarily monocotyledonous plants include maize, sugarcane, sorghum, amaranth, other grasses and sedges. Typical CAM plants are pineapple, epiphytes, succulent xerophytes, hemiepiphytes, lithophytes, terrestrial bromeliads, wetland plants, Mesembryanthemum crystallinum, Dodoneaea viscosa, and Sesuvium portulacastrum. It is possible to express photoassimilation regulation genes from one type of plant in another. For example, C4-cycle enzymes have been introduced into C3 plants. For a review, please see Hausler, et. al. (2002) J of Experimental Botany, Vol. 53, No. 369, pp. 591-607).

5. Yield Increasing or Stress Tolerant Nucleic Acids

There are a number of nucleic acids that may provide improved yield, such as, improved grain yield or biomass. In addition, there are a number of nucleic acids that improve a plants ability to yield under a number of abiotic stresses, such as, drought, salinity, heat, reduced nitrogen, shade tolerance and the like. For example, U.S. Pat. Nos. 7,030,294; 6,686,516; 6,566,511, 5,925,804; 6,833,490; 7,247,770 and US Patent Publication No. 2010/0205692, describe the use of genes of the trehalose pathway for increasing yield and improving stress tolerance. U.S. Pat. Nos. 7,109,033; 7,692,065; 7,732,667 and US Patent Publication Nos. 2003/303589; 2003/299859 describe a number of plant genes for improving a plant's response to stress. Additional genes capable of conferring stress tolerance include, LNT1 gene for improving NUE (WO 2010/031312); GMWRKY54 gene (WO 2009/057061); genes for inhibiting ammonia (US Patent Publication No. 2011/0030099); OsGATA for nitrogen use efficiency (U.S. Pat. No. 7,554,018) and the like.

The terms “stringent conditions” or “stringent hybridization conditions” include reference to conditions under which a nucleic acid molecule will selectively hybridize to a target sequence to a detectably greater degree than other sequences (e.g., at least 2-fold over a non-target sequence), and optionally may substantially exclude binding to non-target sequences. Stringent conditions are sequence-dependent and will vary under different circumstances. By controlling the stringency of the hybridization and/or washing conditions, target sequences can be identified that can be up to 100% complementary to the reference nucleotide sequence. Alternatively, conditions of moderate or even low stringency can be used to allow some mismatching in sequences so that lower degrees of sequence similarity are detected. For example, those skilled in the art will appreciate that to function as a primer or probe, a nucleotide sequence only needs to be sufficiently complementary to the target sequence to substantially bind thereto so as to form a stable double-stranded structure under the conditions employed. Thus, primers or probes can be used under conditions of high, moderate or even low stringency. Likewise, conditions of low or moderate stringency can be advantageous to detect homolog, ortholog and/or paralog sequences having lower degrees of sequence identity than would be identified under highly stringent conditions.

For DNA-DNA hybrids, the Tₘ can be approximated from the equation of Meinkoth and Wahl, Anal. Biochem., 138:267-84 (1984): Tₘ=81.5° C.+16.6 (log M)+0.41 (% GC)−0.61 (% formamide)−500/L; where M is the molarity of monovalent cations, % GC is the percentage of guanosine and cytosine nucleotides in the DNA, % formamide is the percentage of formamide in the hybridization solution, and L is the length of the hybrid in base pairs. The Tₘ is the temperature (under defined ionic strength and pH) at which 50% of a complementary target sequence hybridizes to a perfectly matched probe. Tₘ is reduced by about 1° C. for each 1% of mismatching; thus, Tₘ, hybridization and/or wash conditions can be adjusted to hybridize to sequences of the desired degree of identity. For example, if sequences with >90% identity are sought, the Tₘ can be decreased 10° C. Generally, stringent conditions are selected to be about 5° C. lower than the thermal melting point (Tₘ) for the specific sequence and its complement at a defined ionic strength and pH. However, highly stringent conditions can utilize a hybridization and/or wash at the thermal melting point (Tₘ) or 1, 2, 3 or 4° C. lower than the thermal melting point (Tₘ); moderately stringent conditions can utilize a hybridization and/or wash at 6, 7, 8, 9 or 10° C. lower than the thermal melting point (Tₘ); low stringency conditions can utilize a hybridization and/or wash at 11, 12, 13, 14, 15 or 20° C. lower than the thermal melting point (Tₘ). If the desired degree of mismatching results in a Tₘ of less than 45° C. (aqueous solution) or 32° C. (formamide solution), optionally the SSC concentration can be increased so that a higher temperature can be used. An extensive guide to the hybridization of nucleic acids is found in Tijssen, Laboratory Techniques in Biochemistry and Molecular Biology-Hybridization with Nucleic Acid Probes, part I, chapter 2, “Overview of principles of hybridization and the strategy of nucleic acid probe assays,” Elsevier, New York (1993); Current Protocols in Molecular Biology, chapter 2, Ausubel, et al., eds, Greene Publishing and Wiley-Interscience, New York (1995); and Green & Sambrook, In: Molecular Cloning, A Laboratory Manual, 4th Edition, Cold Spring Harbor Press, Cold Spring Harbor, N.Y. (2012).

Typically, stringent conditions are those in which the salt concentration is less than about 1.5 M Na ion, typically about 0.01 to 1.0 M Na ion concentration (or other salts) at about pH 7.0 to pH 8.3 and the temperature is at least about 30° C. for short probes (e.g., 10 to 50 nucleotides) and at least about 60° C. for longer probes (e.g., greater than 50 nucleotides). Stringent conditions may also be achieved with the addition of destabilizing agents such as formamide or Denhardt's (5 g Ficoll, 5 g polyvinylpyrrolidone, 5 g bovine serum albumin in 500 ml of water). Exemplary low stringency conditions include hybridization with a buffer solution of 30% to 35% formamide, 1 M NaCl, 1% SDS (sodium dodecyl sulfate) at 37° C. and a wash in 1× to 2×SSC (20×SSC=3.0 M NaCl/0.3 M trisodium citrate) at 50° C. to 55° C. Exemplary moderate stringency conditions include hybridization in 40% to 45% formamide, 1 M NaCl, 1% SDS at 37° C. and a wash in 0.5× to 1×SSC at 55° C. to 60° C. Exemplary high stringency conditions include hybridization in 50% formamide, 1 M NaCl, 1% SDS at 37° C. and a wash in 0.1×SSC at 60° C. to 65° C. A further non-limiting example of high stringency conditions include hybridization in 4×SSC, 5×Denhardt's, 0.1 mg/ml boiled salmon sperm DNA, and 25 mM Na phosphate at 65° C. and a wash in 0.1×SSC, 0.1% SDS at 65° C. Another illustration of high stringency hybridization conditions includes hybridization in 7% SDS, 0.5 M NaPO₄, 1 mM EDTA at 50° C. with washing in 2×SSC, 0.1% SDS at 50° C., alternatively with washing in 1×SSC, 0.1% SDS at 50° C., alternatively with washing in 0.5×SSC, 0.1% SDS at 50° C., or alternatively with washing in 0.1×SSC, 0.1% SDS at 50° C., or even with washing in 0.1×SSC, 0.1% SDS at 65° C. Those skilled in the art will appreciate that specificity is typically a function of post-hybridization washes, the relevant factors being the ionic strength and temperature of the final wash solution.

Nucleic acids that do not hybridize to each other under stringent conditions are still substantially identical if the proteins that they encode are substantially identical (e.g., due to the degeneracy of the genetic code).

A nucleic acid sequence is “isocoding with” a reference nucleic acid sequence when the nucleic acid sequence encodes a polypeptide having the same amino acid sequence as the polypeptide encoded by the reference nucleic acid sequence.

As used herein, the term “substantially complementary” (and similar terms) means that two nucleic acid sequences are at least about 50%, 60%, 70%, 75%, 80%, 85%, 90%, 95%, 96%, 97%, 98%, 99% or more complementary. Alternatively, the term “substantially complementary” (and similar terms) can mean that two nucleic acid sequences can hybridize together under high stringency conditions (as described herein).

In representative embodiments, “substantially complementary” means about 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% complementary, or any value or range therein, to a target nucleic acid sequence.

The phrase “hybridizing specifically to” (and similar terms) refers to the binding, duplexing, or hybridizing of a molecule to a particular nucleic acid target sequence under stringent conditions when that sequence is present in a complex mixture (e.g., total cellular DNA or RNA) to the substantial exclusion of non-target nucleic acids, or even with no detectable binding, duplexing or hybridizing to non-target sequences. Selectively hybridizing sequences typically are at least about 40% complementary and are optionally substantially complementary or even completely complementary (i.e., 100% identical) to a nucleic acid sequence.

The term “bind(s) substantially” (and similar terms) as used herein refers to complementary hybridization between a nucleic acid molecule and a target nucleic acid and embraces minor mismatches that can be accommodated by reducing the stringency of the hybridization media to achieve the desired detection of the target nucleic acid sequence.

As used herein, the terms “transformation”, “transfection” and “transduction” refer to the introduction of an exogenous/heterologous nucleic acid (RNA and/or DNA) into a host cell. A cell has been “transformed,” “transfected” or “transduced” with an exogenous/heterologous nucleic acid when such nucleic acid has been introduced or delivered into the cell.

As used herein, the terms “transgenic” and “recombinant” refer to an organism (e.g., a bacterium or plant) that comprises one or more exogenous nucleic acids. Generally, the exogenous nucleic acid is stably integrated within the genome such that at least a portion of the exogenous nucleic acid is passed on to successive generations. The exogenous nucleic acid may be integrated into the genome alone or as part of a recombinant expression cassette. “Transgenic” may be used to designate any organism the genotype of which has been altered by the presence of an exogenous nucleic acid, including those transgenics initially so altered and those created by sexual crosses or asexual propagation from the initial transgenic. As used herein, the term “transgenic” does not encompass the alteration of the genome (chromosomal or extra-chromosomal) by conventional breeding methods or by naturally occurring events such as random cross-fertilization, non-recombinant viral infection, non-recombinant bacterial transformation, non-recombinant transposition or spontaneous mutation.

As used herein, the term “vector” refers to a nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo (i.e., is capable of replication under its own control). The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini. Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. Examples of such markers are disclosed in Messing & Vierra., GENE 19: 259-268 (1982); Bevan et al., NATURE 304:184-187 (1983); White et al., NUCL. ACIDS RES. 18: 1062 (1990); Spencer et al., THEOR. APDL. GENET. 79: 625-631 (1990); Blochinger & Diggelmann, MOL. CELL BIOL. 4: 2929-2931 (1984); Bourouis et al., EMBO J. 2(7): 1099-1104 (1983); U.S. Pat. No. 4,940,935; U.S. Pat. No. 5,188,642; U.S. Pat. No. 5,767,378; and U.S. Pat. No. 5,994,629. A “recombinant” vector refers to a viral or non-viral vector that comprises one or more heterologous nucleotide sequences (i.e., transgenes). Vectors may be introduced into cells by any suitable method known in the art, including, but not limited to, transfection, electroporation, microinjection, transduction, cell fusion, DEAE dextran, calcium phosphate precipitation, lipofection (lysosome fusion), and use of a gene gun or nucleic acid vector transporter.

As used herein, the term “yield reduction” (YD) refers to the degree to which yield is reduced in plants grown under stress conditions. YD is calculated as:

$$\frac{\begin{matrix}yield under non \text{-} stress conditions - \\ yield under stress conditions\end{matrix}}{yield under non \text{-} stress conditions} \times 100$$

The present invention provides compositions and methods useful for increasing yield, increasing yield stability under drought stress conditions, and/or enhancing drought stress tolerance in a plant and/or plant part. Compositions useful for increasing yield, increasing yield stability under drought stress conditions, and/or enhancing drought stress tolerance in a plant and/or plant part may include nucleic acids of the present invention, proteins of the present invention, and/or plants and/or plant parts of the present invention. In some embodiments, a composition and/or method of the present invention may increase seed yield and/or increase harvest index of a plant and/or plant part, optionally when a plant and/or plant part is grown under drought stress conditions.

In some embodiments, a composition and/or method of the present invention may modulate trehalose signaling in a plant and/or plant part. “Modulate,” “modulating,” and grammatical variations thereof as used herein in reference to a trehalose signaling pathway refer to manipulating a component (e.g., a protein) and/or an interaction in the trehalose signaling pathway, such as, for example, increasing or decreasing the availability and/or concentration of a component in a trehalose signaling pathway in a plant and/or plant part. Modulating trehalose signaling in a plant and/or plant part may increase yield, increase yield stability under drought stress conditions, and/or enhance drought stress tolerance in the plant and/or plant part.

In some embodiments, a composition and/or method of the present invention may increase carbon concentration and/or availability, such as, for example, sugar concentration and/or availability (e.g., sucrose concentration and/or availability) in a plant and/or plant part. Some embodiments include increasing carbon concentration and/or availability by modulating trehalose signaling in the plant and/or plant part. In some embodiments, carbon concentration and/or availability may be increased in a particular plant tissue, such as, for example, a reproductive and/or sink tissue (e.g., a flowering tissue and/or seed). In some embodiments, a composition and/or method of the present invention may increase carbon concentration and/or availability in a plant tissue (e.g., a sink tissue) that is growing and/or developing (e.g., the increased carbon concentration and/or availability may be present in a plant tissue during the growth and/or developmental stage of the tissue). In some embodiments, a composition and/or method of the present invention may increase carbon concentration and/or availability in a plant tissue prior to and/or during an early phase of development. In some embodiments, by increasing carbon concentration and/or availability in a plant and/or plant part seed yield and/or harvest index may be increased.

In some embodiments, a composition and/or method of the present invention may be used to overexpress one or more SWEET proteins (e.g., a SWEET 13 protein, a SWEET 14 protein and/or SWEET 15 protein) in a plant and/or plant part. In some embodiments, a composition and/or method of the present invention may be used to overexpress one or more SWEET proteins (e.g., a SWEET 13 protein, a SWEET 14 protein and/or SWEET 15 protein) in a plant and/or plant part and overexpress one more T6PP proteins in the plant and/or plant part. Overexpressing one or more SWEET proteins and/or one or more T6PP proteins may modulate trehalose signaling in a plant and/or plant part and/or may increase carbon concentration and/or availability (e.g., sucrose concentration and/or availability) in a plant and/or plant part. In some embodiments, a composition and/or method of the present invention may be used to decrease the expression of trehalose-6-phosphate (T6P) in a plant and/or plant part.

Some embodiments include overexpressing one or more SWEET proteins, overexpressing one or more T6PP proteins, and/or decreasing the expression and/or concentration (e.g., level) of T6P in a reproductive tissue and/or a sink tissue (e.g., a flowering tissue and/or seed). In some embodiments, a method and/or composition of the present invention may be used to overexpress one or more SWEET proteins, overexpress one or more T6PP proteins, and/or decrease the expression and/or concentration of T6P in a tissue specific manner. For example, one or more SWEET proteins and/or one or more T6PP proteins may be operably linked to a tissue-specific promoter sequence, such as, for example, a flower- and/or seed-specific promoter sequence, to provide tissue-specific expression (e.g., flower- and/or seed-specific expression) of the one or more SWEET proteins and/or one or more T6PP proteins. In some embodiments, providing tissue-specific expression of one or more SWEET proteins and/or one or more T6PP proteins may increase yield, increase yield stability under drought stress conditions, and/or enhance drought stress tolerance in a plant and/or plant part in which said proteins are expressed.

In some embodiments, carbon concentration and/or availability may be increased in a plant tissue by decreasing the expression and/or concentration of T6P in the plant tissue. This may result in an increase in sugar allocation to a particular plant tissue, such as, for example, a reproductive and/or sink tissue.

In some embodiments, a method and/or composition of the present invention may be used to overexpress one or more SWEET proteins and/or one or more T6PP proteins in a specific tissue of a plant and/or plant part (e.g., a reproductive tissue and/or sink tissue) and at a specific stage of development (e.g., during the growth and/or flowering phases of development). In some embodiments, a method and/or composition of the present invention may be used to overexpress one or more SWEET proteins and/or one or more T6PP proteins during the early stage of flowering and/or seed development. In some embodiments, two or more different plant tissues may be targeted for overexpression of one or more SWEET proteins and/or one or more T6PP proteins at one or more stages of development that may be the same and/or different.

In some embodiments, under nondrought stress conditions (e.g., well-watered conditions), a method and/or composition of the present invention may be used to overexpress a SWEET protein, which may increase the sucrose supply in a plant tissue, and may be used to overexpress a T6PP protein and decrease the expression of T6P, which may up-regulate the transcription of a SWEET protein and thereby increase sucrose supply in a plant tissue. This may result in an increased allocation of sucrose to seeds and/or provide an increased yield, such as, for example, by providing an increased seed set and/or increased harvest index.

In some embodiments, under drought stress conditions (e.g., water deficit conditions), a method and/or composition of the present invention may be used to overexpress a SWEET protein, which may increase the sucrose supply in a plant tissue, and may be used to overexpress a T6PP protein and decrease the expression of T6P, which may up-regulate the transcription of a SWEET protein and thereby increase sucrose supply in a plant tissue. This may result in increased yield stability by providing, for example, increased sucrose in a plant tissue, which may support cell division and development and/or may prevent embryo abortion.

In some embodiments, a method and/or composition of the present invention may avoid unintended adverse phenotypes and/or pleotropic effects in a plant and/or plant part.

The present invention encompasses nonnaturally occurring nucleic acids useful for increasing yield, increasing yield stability under drought stress conditions, and/or enhancing drought stress tolerance in a plant and/or plant part.

Nucleic acids of the present invention may comprise, consist essentially of, or consist of a nucleotide sequence that encodes one or more sugar (e.g., sucrose) transporters and/or one or more proteins the expression of which increases the expression, stability and/or activity of one or more sugar (e.g., sucrose) transporters and/or decreases the expression and/or concentration of T6P in a plant tissue. In some embodiments, the nucleic acid comprises, consists essentially of, or consists of:

(a) a nucleotide sequence set forth in any one of SEQ ID NOs: 1 to 11, 29 to 30, 32 to 34 (e.g., SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9, SEQ ID NO:10, SEQ ID NO:11, SEQ ID NO: 20, SEQ ID NO: 30);

(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs: 1 to 11, 29 to 30, 32 to 34;

(c) a nucleotide sequence that encodes a polypeptide comprising the amino acid sequence set forth in any one of SEQ ID NOs: 12 to 16, 31 (e.g., SEQ ID NO:12, SEQ ID NO:13, SEQ ID NO:14, SEQ ID NO:15, SEQ ID NO:16);

(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs: 12 to 16, 31;

(e) a nucleotide sequence that is complementary to the nucleotide sequence of any one of (a) to (d) above;

(f) a nucleotide sequence that hybridizes to the nucleotide sequence of any one of (a) to (e) above under stringent hybridization conditions;

(g) a functional fragment of any one of (a) to (f) above, wherein the functional fragment encodes a sucrose transporter;

(h) a functional fragment of any one of (a) to (f) above, wherein the functional fragment encodes a polypeptide that comprises five alpha-helical transmembrane domains;

(i) a functional fragment of any one of (a) to (f) above, wherein the functional fragment encodes a polypeptide that comprises six alpha-helical transmembrane domains;

(j) a functional fragment of any one of (a) to (f) above, wherein the functional fragment encodes a polypeptide that comprises seven alpha-helical transmembrane domains, and any combination thereof.

In some embodiments, a nonnaturally occurring nucleic acid of the invention may encode two or more SWEET proteins (e.g., a SWEET 13 protein, a SWEET 14 protein and/or SWEET 15 protein). In some embodiments, the nonnaturally occurring nucleic acid may encode the same protein (e.g., two copies of a SWEET 13 protein) and/or may encode two different proteins (e.g., two different SWEET 13 proteins). For example, in some embodiments, a nucleic acid may comprise at least two nucleotide sequences that are at least 70% identical to a nucleotide sequence of any one of SEQ ID NOs: 1 to 11, 29 to 30, 32 to 34, a nucleotide sequence encoding a polypeptide comprising an amino acid sequence that is at least 70% identical to the amino acid sequence set forth in any one of SEQ ID NOs: 12 to 16, 31, or any combination thereof. In some embodiments, the nucleic acid comprises a nucleotide sequence that is at least 70% identical to the nucleotide sequence of any one of SEQ ID NOs: 1 to 2 or 6 to 11 or encodes a polypeptide comprising an amino acid sequence that is at least 70% identical to the amino acid sequence set forth in any one of SEQ ID NOs: 12 or 14 to 16, and a nucleotide sequence that is at least 70% identical to the nucleotide sequence of any one of SEQ ID NOs: 3 to 5 or encodes a polypeptide comprising an amino acid sequence that is at least 70% identical to the amino acid sequence set forth in SEQ ID NO:13.

In some embodiments, a nonnaturally occurring nucleic acid encoding two or more SWEET proteins (e.g., a SWEET 13 protein, a SWEET 14 protein and/or SWEET 15 protein) may allow for the sugar concentration and/or availability to be modified (e.g., increased) in two or more different tissues in a plant and/or plant part expressing the nonnaturally occurring nucleic acid compared to a plant and/or plant part that does not express the nonnaturally occurring nucleic acid. For example, a SWEET 13 protein (e.g., SWEET 13a) may affect and/or modify the sugar concentration and/or availability in at least one tissue different than a different SWEET 13 protein (e.g., SWEET 13c), oa SWEET 14 protein (e.g., SWEET 14b) or a SWEET 15 protein (e.g., SWEET15b). In some embodiments, a nonnaturally occurring nucleic acid encoding two or more different SWEET proteins may provide an increase in sugar concentration and/or availability in a plant or plant tissue expressing the nonnaturally occurring nucleic acid by overexpressing the two or more sugar transporters as compared to the sugar concentration and/or availability in a plant or plant tissue due to the overexpression of one SWEET protein. In some embodiments, the increase in sugar concentration and/or availability in a plant tissue may be due to modulating trehalose signaling in the plant tissue by expressing the nonnaturally occurring nucleic acid encoding two or more SWEET proteins.

Some embodiments include that a nonnaturally occurring nucleic acid may encode a T6PP protein and/or a functional fragment thereof. In some embodiments, the nucleic acid comprises a nucleotide sequence that is at least 70%, 75%, 80%, 85%, 90%, 91%, 92%, 93%, 94%, 95%, 96%, 97%, 98%, 99%, 99.5% or more identical to the nucleotide sequence of one of SEQ ID NOs: 17 to 20, a nucleotide sequence that encodes a polypeptide comprising the amino acid sequence of any one of SEQ ID NOs: 21 to 24, and/or a functional fragment thereof. Thus, in some embodiments, a nonnaturally occurring nucleic acid may encode a T6PP protein and at least one SWEET 13 protein, SWEET 14 protein and/or SWEET 15 protein.

Some embodiments include a nonnaturally occurring nucleic acid comprising a promoter sequence. In some embodiments, the nucleic acid comprises a nucleotide sequence that is at least 70%, 75%, 80%, 85%, 90%, 91%, 92%, 93%, 94%, 95%, 96%, 97%, 98%, 99%, 99.5% or more identical to the nucleotide sequence of one of SEQ ID NOs: 32 to 34 and/or a functional fragment thereof.

Nucleic acids of the present invention may comprise any suitable promoter sequence(s), including, but not limited to, constitutive promoters, tissue-specific promoters, chemically inducible promoters, wound-inducible promoters, stress-inducible promoters and developmental stage-specific promoters. In some embodiments, a nucleic acid of the present invention may be operably linked to a promoter that is the same as or substantially identical to a native promoter, such as, for example, a promoter endogenous to the plant and/or plant part the nucleic acid is to be expressed in or is endogenous to the polynucleotide to be expressed. Some embodiments include that a native promoter is the same as or substantially identical to the promoter operably linked to an endogenous nucleic acid encoding a protein substantially identical to the protein encoded by a nucleic acid of the present invention. For example, a nucleic acid of the present invention encoding a SWEET protein may be operably linked to a promoter that is the same as or substantially the same as a promoter that is operably linked to an endogenous SWEET gene (e.g., that encodes the same or a different SWEET protein). In some embodiments, a nucleic acid of the present invention encoding a SWEET protein (e.g., SWEET 14b) may be operably linked to a SWEET 14 promoter (e.g. SWEET 14b promoter).

In some embodiments, the nucleic acid comprises one or more constitutive promoter sequences. For example, the nucleic acid may comprise one or more CaMV 19S, CaMV 35S, Arabidopsis At6669, maize H3 histone, rice actin 1, actin 2, rice cyclophilin, nos, Adh, sucrose synthase, pEMU, GOS2, constitutive root tip CT2, and/or ubiquitin (e.g., maize Ubi) promoter sequences. Examples of suitable promoters are disclosed in U.S. Pat. Nos. 5,352,605, 5,641,876, 5,604,121, 6,040,504 and 7,166,770; WO 93/07278; WO 01/73087; EP 0342926; Binet et al., PLANT Sci. 79:87-94 (1991); Christensen et al., PLANT MOLEC. BIOL. 12: 619-632 (1989); Ebert et al., PROC. NATL. ACAD. SCI USA 84:5745-5749 (1987); Norris et al., PLANT MOLEC. BIOL. 21:895-906 (1993); Walker et al., PROC. NATL. ACAD. SCI. USA 84:6624-6629 (1987); Wang et al., MOL. CELL. BIOL. 12:3399-3406 (1992); and Yang & Russell, PROC. NATL. ACAD. SCI. USA 87:4144-4148 (1990). Thus, in some embodiments, the nucleic acid comprises one or more of the nucleotide sequences described in (a) to (j) above operably linked to one or more constitutive promoters.

In some embodiments, the nucleic acid comprises one or more tissue-specific promoter sequences. For example, the nucleic acid may comprise one or more flower-, leaf-, ligule-, node-, internode-, panicle-, root-, seed-, sheath-, stem-, and/or vascular bundle-specific promoter sequences. Examples of suitable promoters are disclosed in U.S. Pat. Nos. 5,459,252, 5,604,121, 5,625,136, 6,040,504 and 7,579,516; EP 0452269; WO 93/07278; Czako et al., MOL. GEN. GENET. 235:33-40 (1992); Hudspeth & Grula, PLANT MOLEC. BIOL. 12:579-589 (1989); de Framond, FEBS 290:103-106 (1991); Jeong et al. PLANT PHYSIOL. 153:185-197 (2010); and KIM ET AL. PLANT CELL 18:2958-2970 (2006). Thus, in some embodiments, the nucleic acid comprises one or more of the nucleotide sequences described in (a) to (j) above operably linked to one or more tissue-specific promoters.

In some embodiments, a nucleic acid of the present invention may comprise, consist essentially of, or consist of a tissue-specific promoter sequence, such as, for example, a flower-, seed-, endosperm-, embryo-, panicle-, and/or node-specific promoter sequence. This may provide for the nucleic acid to be expressed in a flower, seed, endosperm, embryo, panicle, and/or node of the plant or plant part and/or may provide for an increase in sugar (e.g., sucrose) concentration and/or availability in a flower, seed, endosperm, embryo, panicle, and/or node of the plant or plant part expressing the nucleic acid. In some embodiments, the tissue-specific promoter sequence may be an OsMADS promoter (e.g., an OsMADS6 promoter or an OsMADS7 promoter). MADS is a class of transcriptional regulator genes defined by founding members MCM1, AGAMOUS, DEFICIENS and Serum Response Factor. Expression control by OsMADS6 promoter provides for expression in reproductive and/or sink tissues, such as, for example, in corn in ear nodes, ear vasculature and spikelet tissues. OsMADS7 promoter provides for significant expression in ovule and developing maize kernel. In contrast, the OsMADS7 promoter does not drive significant expression in non-flowering tissues, such as, ear node, tassel, leaf or silk. Example OsMADS promoters include, but are not limited to, those described in International Publication No. WO 2005/102034, the contents of which are incorporated herein by reference in its entirety. In some embodiments, the tissue-specific promoter sequence may be a SWEET promoter operably linked to a SWEET gene; for example a SWEET13 a promoter (SEQ ID NO: 32); a SWEET 14b promoter (SEQ ID NO: 33) or a SWEET 15b promoter (SEQ ID NO: 34). In some embodiments the promoter is a drought inducible embryo specific promoter. Examples of drought inducible embryo specific promoters are promoters driving the gt1—grassy tillers1 homeobox-transcription factor GRMZM2G005624; NAC-transcription factor 25 GRMZM2G27379 and AP2-EREBP-transcription factor 162; APETALA2-EREBP GRMZM2G059939.

In some embodiments, the nucleic acid comprises one or more chemically inducible promoter sequences. Examples of suitable promoters are disclosed in U.S. Pat. Nos. 5,614,395, 5,789,156 and 5,814,618; EP 0332104; WO 97/06269; WO 97/06268; Aoyama et al., PLANT J. 11:605-612 (1997); De Cosa et al. NAT. BIOTECHNOL. 19:71-74 (2001); Daniell et al. BMC BIOTECHNOL. 9:33 (2009); Gatz et al. MOL. GEN. GENET. 227, 229-237 (1991); Gatz, CURRENT OPINION BIOTECHNOL. 7:168-172 (1996); Gatz, ANN. REV. PLANT PHYSIOL. PLANT MOL. BIOL. 48:89-108 (1997); Li et al., GENE 403:132-142 (2007); Li et al., MoL BIOL. REP. 37:1143-1154 (2010); McNellis et al. PLANT J. 14, 247-257 (1998); Muto et al. BMC BIOTECHNOL. 9:26 (2009); Schena et al. PROC. NATL. ACAD. SCI. USA 88, 10421-10425 (1991); Surzycki et al. BIOLOGICALS 37:133-138 (2009); and Walker et al. PLANT CELL REP. 23:727-735 (2005). Thus, in some embodiments, the nucleic acid comprises one or more of the nucleotide sequences described in (a) to (j) above operably linked to one or more chemically inducible promoters.

In some embodiments, the nucleic acid comprises one or more wound-inducible promoter sequences. Examples of suitable promoters are disclosed in Stanford et al., MOL. GEN. GENET. 215:200-208 (1989); Xu et al., PLANT MOLEC. BIOL. 22:573-588 (1993); Logemann et al., PLANT CELL 1:151-158 (1989); Rohrmeier & Lehle, PLANT MOLEC. BIOL. 22:783-792 (1993); Firek et al., PLANT MOLEC. BIOL. 22:129-142 (1993); and Warner et al., PLANT J. 3:191-201 (1993). Thus, in some embodiments, the nucleic acid comprises one or more of the nucleotide sequences described in (a) to (j) above operably linked to one or more wound-inducible promoters.

In some embodiments, the nucleic acid comprises one or more stress-inducible promoter sequences. For example, the nucleic acid may comprise one or more drought stress-inducible, salt stress-inducible, heat stress-inducible, light stress-inducible and/or osmotic stress-inducible promoter sequences. Thus, in some embodiments, the nucleic acid comprises one or more of the nucleotide sequences described in (a) to (j) above operably linked to one or more stress-inducible promoters. In some embodiments, the nucleic acid comprises a drought stress-inducible promoter sequence.

In some embodiments, the nucleic acid comprises one or more developmental stage-specific promoter sequences. For example, the nucleic acid may comprise a promoter sequence that drives expression prior to and/or during the seedling, tillering, panicle initiation, panicle differentiation, reproductive (e.g., flowering, pollination, fertilization), and/or grain filling stage(s) of development. Thus, in some embodiments, the nucleic acid comprises one or more of the nucleotide sequences described in (a) to (j) above operably linked to one or more developmental-stage specific promoters. In some embodiments, the nucleic acid comprises a promoter sequence that drives expression prior to and/or during the seedling and/or reproductive stage(s) of development.

In some embodiments, the nucleic acid comprises one or more promoters useful for expression in bacteria and/or yeast. For example, the nucleic acid may comprise one or more yeast promoters associated with phosphoglycerate kinase (PGK), glyceraldehyde-3-phosphate dehydrogenase (GAP), triose phosphate isomerase (TPI), galactose-regulon (GAL1, GAL10), alcohol dehydrogenase (ADH1, ADH2), phosphatase (PHO5), copper-activated metallothionine (CUP1), MFα1, PGK/α2 operator, TPI/α2 operator, GAP/GAL, PGK/GAL, GAP/ADH2, GAP/PHOS, iso-1-cytochrome c/glucocorticoid response element (CYC/GRE), phosphoglycerate kinase/angrogen response element (PGK/ARE), transcription elongation factor EF-1α (TEF1), triose phosphate dehydrogenase (TDH3), phosphoglycerate kinase 1 (PGK1), pyruvate kinase 1 (PYK1), and/or hexose transporter (HXT7). Likewise, the nucleic acid may comprise any bacterial L-arabinose inducible (araBAD, P_{BAD}) promoter, lac promoter, L-rhamnose inducible (rhaP_{BAD}) promoter, T7 RNA polymerase promoter, trc promoter, we promoter, lambda phage promoter (p_{L}, p_{L}-9G-50), anydrotetracycline-inducible (tetA) promoter, trp, lpp, phoA, recA, proU, cst-1, cadA, nar, lpp-lac, cspA, T7-lac operator, T3-lac operator, T4 gene 32, T5-lac operator, nprM-lac operator, Vhb, Protein A, corynebacterial-E. coli like promoters, thr, horn, diphtheria toxin promoter, sig A, sig B, nusG, SoxS, katb, α-amylase (Parry), Ptms, P43 (comprised of two overlapping RNA polymerase G factor recognition sites, GA, GB), Ptms, P43, rplK-rplA, ferredoxin promoter, and/or xylose promoter. Examples of suitable promoters are disclosed in Hannig et al. TRENDS BIOTECHNOL. 16:54-60 (1998); Partow et al. YEAST 27:955-964 (2010); Romanos et al. YEAST 8:423-488 (1992); Srivastava et al., PROTEIN EXPR. PURIF. 40:221-229 (2005); Terpe, APPL. MICROBIOL, BIOTECHNOL. 72:211-222 (2006). Thus, in some embodiments, the nucleic acid comprises one or more of the nucleotide sequences described in (a) to (j) above operably linked to one or more yeast and/or bacterial promoters.

Nucleic acids of the present invention may comprise any suitable termination sequence(s). For example, the nucleic acid may comprise a termination sequence comprising a stop signal for RNA polymerase and a polyadenylation signal for polyadenylase. Thus, the nucleic acid comprises one or more of the nucleotide sequences described in (a) to (j) above operably linked to one or more termination sequences.

Nucleic acids of the present invention may comprise any suitable expression-enhancing sequence(s). For example, the nucleic acid may comprise one or more intron sequences (e.g., Adhl and/or bronzel) and/or viral leader sequences (from tobacco mosaic virus (TMV), tobacco etch virus (TEV), maize chlorotic mottle virus (MCMV), maize dwarf mottle virus (MDMV) or alfalfa mosaic virus (AMV), for example) that enhance expression of associated nucleotide sequences. Examples of suitable sequences are disclosed in Allison et al. VIROLOGY 154:9-20 (1986); Della-Cioppa et al. PLANT PHYSIOL. 84:965-968 (1987); Elroy-Stein et al. PROC. NATL. ACAD. SCI. USA 86:6126-6130 (1989); Gallie et al., GENE 165:233-238 (1995); Gallie et al. NUCLEIC ACIDS RES. 15:8693-8711 (1987); Gallie et al. NUCLEIC ACIDS RES. 15:3257-3273 (1987); Gallie et al. NUCLEIC ACIDS RES. 16:883-893 (1988); Gallie et al. NUCLEIC ACIDS RES. 20:4631-4638 (1992); Jobling et al. NATURE 325:622-625 (1987); Lommel et al. VIROLOGY 81:382-385 (1991); Skuzeski et al., PLANT MOLEC. BIOL. 15:65-79 (1990). Thus, the nucleic acid comprises one or more of the nucleotide sequences described in (a) to (j) above operably linked to one or more expression-enhancing sequences.

Nucleic acids of the present invention may comprise any suitable transgene(s), including, but not limited to, transgenes that encode gene products that provide enhanced abiotic stress tolerance (e.g., enhanced drought stress tolerance, enhanced osmotic stress tolerance, enhanced salt stress tolerance and/or enhanced temperature stress tolerance), herbicide-resistance (e.g., enhanced glyphosate-, Sulfonylurea-, imidazolinione-, dicamba-, glufisinate-, phenoxy proprionic acid-, cycloshexome-, traizine-, benzonitrile-, and/or broxynil-resistance), pest-resistance and/or disease-resistance.

Nucleic acids of the present invention may encode any suitable epitope tag, including, but not limited to, poly-Arg tags (e.g., RRRRR and RRRRRR) and poly-His tags (e.g., HHHHHH).

In some embodiments, the nucleic acid comprises a nucleotide sequence encoding a poly-Arg tag, a poly-His tag, a FLAG tag (i.e., DYKDDDDK), a Strep-tag II™ (GE Healthcare, Pittsburgh, Pa., USA) (i.e., WSHPQFEK), and/or a c-myc tag (i.e., EQKLISEEDL).

Nucleic acids of the present invention may comprise any suitable number of nucleotides. In some embodiments, the nucleic acid is 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000 or more nucleotides in length. In some embodiments, the nucleic acid is less than about 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000 nucleotides in length. In some embodiments, the nucleic acid is about 1500, 1550, 1600, 1650, 1700, 1750, 1800, 1850, 1900, 1950, 2000, 2050, 2100, 2150, 2200, 2250, 2300, 2350, 2400, 2450, 2500, 2550, 2600, 2650, 2700, 2750, 2800, 2850, 2900, 2950, 3000, 3050, 3100, 3150, 3200, 3250, 3300, 3350, 3400, 3450, 3500, 3550, 3600, 3650, 3700, 3750, 3800, 3850, 3900, 3950, 4000 nucleotides in length.

A nucleic acid of the present invention may be codon optimized. In some embodiments, a nucleic acid of the present invention may be codon optimized for expression in bacteria, viruses, fungi and/or plants. Codon optimization is well known in the art and involves modification of a nucleotide sequence for codon usage bias using species-specific codon usage tables. The codon usage tables are generated based on a sequence analysis of the most highly expressed genes for the species of interest. When the nucleotide sequences are to be expressed in the nucleus, the codon usage tables are generated based on a sequence analysis of highly expressed nuclear genes for the species of interest. The modifications of the nucleotide sequences are determined by comparing the species specific codon usage table with the codons present in the native polynucleotide sequences. As is understood in the art, codon optimization of a nucleotide sequence results in a nucleotide sequence having less than 100% identity (e.g., 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, and the like) to 30 the native nucleotide sequence but which still encodes a polypeptide having the same function as that encoded by the original, native nucleotide sequence. Thus, in some embodiments of the present invention, the nucleic acid molecule may be codon optimized for expression in a particular species of interest (e.g., a plant such as maize, soybean, sugar cane, sugar beet, rice or wheat).

Because expression levels may also be dependent on GC content, nucleic acids of the present invention may also be GC-optimized. That is, the nucleotide sequences of nucleic acids of the present invention may be selectively altered to optimize their GC content for increased expression in the desired organism. For example, because microbial nucleotide sequences that have low GC contents may express poorly in plants due to the existence of ATTTA motifs that may destabilize messages and/or AATAAA motifs that may cause inappropriate polyadenylation, expression in plants may be enhanced by increasing GC content to at least about 35%, 40%, 45%, 50%, 55%, 60%, 65%, 70%, 75% or more.

In some embodiments, nucleic acids of the present invention are isolated nucleic acids.

The present invention also encompasses expression cassettes comprising one or more nucleic acid(s) of the present invention. In some embodiments, the expression cassette comprises a nucleic acid that confers at least one property (e.g., resistance to a selection agent) that can be used to detect, identify or select transformed plant cells and tissues.

An expression cassette of the present invention may also include nucleotide sequences that encode other desired traits. Such desired traits can be other nucleotide sequences which confer other agriculturally desirable traits. Such nucleotide sequences can be stacked with any combination of nucleotide sequences to create plants, plant parts or plant cells having the desired phenotype. Stacked combinations can be created by any method including, but not limited to, cross breeding plants by any conventional methodology, or by genetic transformation. If stacked by genetically transforming the plants, nucleotide sequences encoding additional desired traits can be combined at any time and in any order. For example, a transgenic plant comprising one or more desired traits can be used as the target to introduce further traits by subsequent transformation. The additional nucleotide sequences can be introduced simultaneously in a co-transformation protocol with a nucleotide sequence, nucleic acid molecule, nucleic acid construct, and/or composition of the invention, provided by any combination of expression cassettes. For example, if two nucleotide sequences will be introduced, they can be incorporated in separate cassettes (trans) or can be incorporated on the same cassette (cis). Expression of the nucleotide sequences can be driven by the same promoter or by different promoters. It is further recognized that nucleotide sequences can be stacked at a desired genomic location using a site-specific recombination system. See, e.g., Int'l Patent Application Publication Nos. WO 99/25821; WO 99/25854; WO 99/25840; WO 99/25855 and WO 99/25853. In representative embodiments, a nucleic acid molecule, expression cassette or vector of the invention can comprise a transgene that confers resistance to one or more herbicides, optionally glyphosate-, sulfonylurea-, imidazolinione-, dicamba-, glufisinate-, phenoxy proprionic acid-, cycloshexome-, traizine-, benzonitrile-, and/or broxynil-resistance; a transgene that confers resistance to one or more pests, optionally bacterial-, fungal, gastropod-, insect-, nematode-, oomycete-, phytoplasma-, protozoa-, and/or viral-resistance, and/or a transgene that confers resistance to one or more diseases. In some embodiments, a nucleic acid, expression cassette and/or vector of the present invention may comprise one or more transgenes that confer tolerance to one or more additional abiotic stresses. Thus, for example, transgenes that confer an additional abiotic stress tolerance may confer tolerance to an abiotic stress including, but not limited to, cold temperatures (e.g., freezing and/or chilling temperatures), heat or high temperatures, drought, flooding, high light intensity, low light intensity, extreme osmotic pressures, extreme salt concentrations, high winds, ozone, poor edaphic conditions (e.g., extreme soil pH, nutrient-deficient soil, compacted soil, etc.), and/or combinations thereof.

The present invention also encompasses vectors comprising one or more nucleic acid(s) and/or expression cassette(s) of the present invention. In some embodiments, the vector is a pSTK, pROKI, pBin438, pCAMBIA (e.g., pCAMBIA1302, pCAMBIA2301, pCAMBIA1301, pCAMBIA1391-Xa, pCAMBIA1391-Xb) (CAMBIA Co., Brisbane, Australia) or pBI121 vector.

In some embodiments, an expression cassette and/or vector may comprise a nucleotide sequence that encodes a SWEET protein (e.g., a SWEET 13 protein, a SWEET 14 protein and/or SWEET 15 protein). In some embodiments, the nucleotide sequence may comprise:
(a) a nucleotide sequence set forth in any one of SEQ ID NOs:1 to 11, 29 to 30, 32 to 34, 32-34;
(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs:1 to 11, 29 to 30, 32 to 34, 32-34;
(c) a nucleotide sequence that encodes a polypeptide comprising the amino acid sequence set forth in any one of SEQ ID NOs:12 to 16, 31;
(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs:12 to 16, 31;
(e) a nucleotide sequence that is complementary to the nucleotide sequence of any one of (a) to (d) above;
(f) a nucleotide sequence that hybridizes to the nucleotide sequence of any one of (a) to (e) above under stringent hybridization conditions;
(g) a functional fragment of any one of (a) to (f) above, wherein the functional fragment encodes a sucrose transporter;
(h) a functional fragment of any one of (a) to (f) above, wherein the functional fragment encodes a polypeptide that comprises five alpha-helical transmembrane domains;
(i) a functional fragment of any one of (a) to (f) above, wherein the functional fragment encodes a polypeptide that comprises six alpha-helical transmembrane domains;
(j) a functional fragment of any one of (a) to (f) above, wherein the functional fragment encodes a polypeptide that comprises seven alpha-helical transmembrane domains; and any combination thereof. The nucleotide sequence may be operably linked to a promoter. In some embodiments, the promoter may comprise a tissue-specific promoter sequence, such as, for example, a flower-, seed-, endosperm-, embryo-, panicle-, and/or node-specific promoter sequence.

In some embodiments, an expression cassette and/or vector may comprise two or more nucleotide sequences that encode the same and/or different SWEET proteins (e.g., a SWEET 13 protein, a SWEET 14 protein and/or SWEET 15 protein). The two or more nucleotide sequences may be operably linked to the same promoter, separate promoters, or any combination thereof.

When separate promoters are used for the two or more nucleotides, the same and/or different promoters may be used.

Some embodiments include that an expression cassette and/or vector comprises at least two nucleotide sequences that are each independently selected from the group consisting of:
(a) a nucleotide sequence set forth in any one of SEQ ID NOs:1 to 11, 29 to 30, 32 to 34;
(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs:1 to 11, 29 to 30, 32 to 34;
(c) a nucleotide sequence that encodes a polypeptide comprising the amino acid sequence set forth in any one of SEQ ID NOs:12 to 16, 31;
(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs:12 to 16, 31;
(e) a nucleotide sequence that is complementary to the nucleotide sequence of any one of (a) to (d) above;
(f) a nucleotide sequence that hybridizes to the nucleotide sequence of any one of (a) to (e) above under stringent hybridization conditions;
(g) a functional fragment of any one of (a) to (f) above, wherein the functional fragment encodes a sucrose transporter;
(h) a functional fragment of any one of (a) to (f) above, wherein the functional fragment encodes a polypeptide that comprises five alpha-helical transmembrane domains;
(i) a functional fragment of any one of (a) to (f) above, wherein the functional fragment encodes a polypeptide that comprises six alpha-helical transmembrane domains; and
(j) a functional fragment of any one of (a) to (f) above, wherein the functional fragment encodes a polypeptide that comprises seven alpha-helical transmembrane domains.

In some embodiments, an expression cassette and/or vector may comprise at least one nucleotide sequence that is at least 70% identical to the nucleotide sequence of any one of SEQ ID NOs:1 to 2 or 6 to 11 or that encodes a polypeptide comprising an amino acid sequence that is at least 70% identical to the amino acid sequence set forth in any one of SEQ ID NOs:12 or 14 to 16, and at least one nucleotide sequence that is at least 70% identical to the nucleotide sequence of any one of SEQ ID NOs:3 to 5 or that encodes a polypeptide comprising an amino acid sequence that is at least 70% identical to the amino acid sequence set forth in SEQ ID NO: 13.

Some embodiments include that an expression cassette and/or vector may comprise a nucleotide sequence that encodes a SWEET protein (e.g., a a SWEET 13 protein, a SWEET 14 protein and/or a SWEET 15 protein) and a nucleotide sequence that encodes a T6PP protein.

The expression cassette and/or vector may comprise a nucleotide sequence that encode one or more T6PP proteins. In some embodiments, an expression cassette and/or vector may comprise a nucleotide sequence that is at least 70% identical to the nucleotide sequence of any one of SEQ ID NOs: 17-20 and/or to the nucleotide sequence of one or more of the nucleotide sequences that encode a polypeptide comprising the amino acid sequence of any one of SEQ ID NOs:21 to 24. The nucleotide sequence encoding a T6PP protein may be operably linked to a promoter. In some embodiments, the nucleotide sequence encoding a T6PP protein and the nucleotide sequence encoding a SWEET protein may be operably linked to the same or separate promoters. When a separate promoter is used for the nucleotide sequence encoding a T6PP protein, the same promoter and/or a different promoter may be used as that for a nucleotide sequence encoding a SWEET protein.

The present invention also encompasses transgenic cells/organisms comprising one or more nucleic acids, expression cassettes, and/or vectors of the present invention. In some embodiments, the transgenic organism is a bacteria, virus, fungus, plant, or plant part. In some embodiments, the transgenic cell is a fungal spore or fungal gamete. In some embodiments, the transgenic cell is a propagating plant cell, such as an egg cell or sperm cell. In some embodiments, the transgenic cell is a non-propagating plant cell.

The present invention also encompasses nonnaturally occurring proteins useful for increasing yield, increasing yield stability under drought conditions, and/or enhancing drought stress tolerance in a plant or plant part.

Proteins of the present invention may comprise an amino acid sequence the expression of which increases yield, increasing yield stability (such as, for example, under drought conditions), and/or enhances drought stress tolerance in a plant or plant part. In some embodiments, the protein is a sugar (e.g., sucrose) transporter protein, such as, for example, a SWEET 13 protein, a SWEET 14 protein and/or a SWEET 15 protein as described herein. In some embodiments, the protein is a protein capable of increasing the expression, stability and/or activity of one or more sugar (e.g., sucrose) transporters and/or decreasing the expression and/or concentration of T6P in a plant and/or plant part. In some embodiments, the protein capable of increasing the expression, stability and/or activity of one or more sugar (e.g., sucrose) transporters and/or decreasing the expression and/or concentration of T6P in a plant and/or plant part may be a T6PP protein as described herein. In some embodiments, the expression of a protein of the present invention in a plant and/or plant part may modulate trehalose signaling and/or increase sugar concentration and/or availability in a plant and/or plant part.

In some embodiments, the protein is an isolated protein.

Polypeptides and fragments of the invention can be modified for in vivo use by the addition, at the amino- and/or carboxyl-terminal ends, of a blocking agent to facilitate survival of the relevant polypeptide in vivo. This can be useful in those situations in which the peptide termini tend to be degraded by proteases prior to cellular uptake. Such blocking agents can include, without limitation, additional related or unrelated peptide sequences that can be attached to the amino and/or carboxyl terminal residues of the peptide to be administered. For example, one or more non-naturally occurring amino acids, such as D-alanine, can be added to the termini. Alternatively, blocking agents such as pyroglutamic acid or other molecules known in the art can be attached to the amino and/or carboxyl terminal residues, or the amino group at the amino terminus or carboxyl group at the carboxyl terminus can be replaced with a different moiety. Additionally, the peptide terminus can be modified, e.g., by acetylation of the N-terminus and/or amidation of the C-terminus. Likewise, the peptides can be covalently or noncovalently coupled to pharmaceutically acceptable “carrier” proteins prior to administration.

A protein of the present invention may comprise any suitable epitope tag, including, but not limited to, poly-Arg tags (e.g., RRRRR and RRRRRR) and poly-His tags (e.g., HHHHHH).

In some embodiments, the nucleic acid comprises a nucleotide sequence encoding a poly-Arg tag, a poly-His tag, a FLAG tag (i.e., DYKDDDDK), a Strep-tag II™ (GE Healthcare, Pittsburgh, Pa., USA) (i.e., WSHPQFEK), and/or a c-myc tag (i.e., EQKLISEEDL).

A protein of the present invention may comprise any suitable number of amino acids. In some embodiments, the protein is 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 450, 500, 550, 600, 650, 700, 750, 800, 850, 900, 950, 1000, 1050, 1100, 1150, 1200, 1200, 1250, 1300, 1350, 1400, 1450, 1500 or more amino acids in length. In some embodiments, the protein is less than about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 450, 500, 550, 600, 650, 700, 750, 800, 850, 900, 950, 1000, 1050, 1100, 1150, 1200, 1200, 1250, 1300, 1350, 1400, 1450, or 1500 amino acids in length. In some embodiments, the protein is about 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, 400, 450, 500, 550, 600, 650, 700, 750, 800, 850, 900, 950, 1000, 1050, 1100, 1150, 1200, 1200, 1250, 1300, 1350, 1400, 1450, or 1500 amino acids in length.

A protein of the present invention may be produced using any suitable means, including, but not limited to, expression of nucleic acids of the present invention in a transgenic organism.

In some embodiments, a protein of the present invention may be produced using a transgenic bacterium/fungus expressing one or more nucleic acids of the present invention under the control of one or more heterologous regulatory elements (e.g., the nucleotide sequence of SEQ ID NO: 1 under the control of a constitutive promoter suitable for use in Bt).

A protein of the present invention may possess any suitable activity in increasing and/or decreasing the amount of a sugar present and/or available in a plant and/or plant part. In some embodiments, a protein of the present invention may be overexpressed and may increase the amount of a sugar (e.g., sucrose) present and/or available for use (such as, for example, for use as an energy source) in a plant tissue, such as, for example, a flowering tissue. Some embodiments of the present invention involve overexpressing a SWEET protein (e.g., a SWEET 13 protein, a SWEET 14 protein and/or a SWEET 15 protein).

Nucleic acids and proteins of the present invention may be expressed in any suitable cell/organism, including, but not limited to, plants, bacteria, viruses and fungi. In some embodiments, the nucleic acid/protein is expressed in a monocot plant or plant part (e.g., in rice, maize, wheat, barley, oats, rye, millet, sorghum, fonio, sugar cane, bamboo, durum, kamut, triticale, secale, einkorn, spelt, emmer, teff, milo, flax, banana, ginger, onion, lily, daffodil, iris, amaryllis, orchid, canna, bluebell, tulip, garlic, gramma grass, Tripsacum sp., or teosinte). In some embodiments, the nucleic acid/protein is expressed in a dicot plant or plant part (e.g., in buckwheat, cotton, potato, quinoa, soybean, sugar beet, sunflower, tobacco or tomato).

Once a nucleotide sequence has been introduced into a particular cell/organism, it may be propagated in that species using traditional methods. Furthermore, once the nucleotide sequence has been introduced into a particular plant variety, it may be moved into other varieties (including commercial varieties) of the same species.

In some embodiments, the present invention provides a method of identifying a plant and/or plant part having increased yield, increased yield stability, and/or enhanced drought stress tolerance, the method comprising detecting, in a plant and/or plant part, one or more nucleic acids that comprises one or more of the nucleotide sequences set forth in any one of SEQ ID NOs:1 to 11, 29 to 30, 32 to 34, one or more nucleotide sequences that encodes a polypeptide comprising the amino acid sequence of any one of SEQ ID NOs:12 to 16, 31, one or more nucleotide sequences that is at least 70%, 75%, 80%, 85%, 90%, 91%, 92%, 93%, 94%, 95%, 96%, 97%, 98%, 99%, 99.5% or more identical to the nucleotide sequence set forth in any one of SEQ ID NOs:1 to 11, 29 to 30, 32 to 34, one or more nucleotide sequences that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence of any one of SEQ ID NOs:12 to 16, 31, one or more nucleotide sequences that is complementary to one of the aforementioned nucleotide sequences, one or more nucleotide sequences that specifically hybridize to one of the aforementioned nucleotide sequences under stringent hybridization conditions, and/or a functional fragment of one of the aforementioned nucleotide sequences. In some embodiments, the present invention provides a method of producing a plant having increased yield, increased yield stability, and/or enhanced drought stress tolerance, the method comprising detecting, in a plant part, one or more nucleic acids comprising one or more of the nucleotide sequences set forth in any one of SEQ ID NOs:1 to 11, 29 to 30, 32 to 34, one or more nucleotide sequences that encodes a polypeptide comprising the amino acid sequence of any one of SEQ ID NOs:12 to 16, 31, one or more nucleotide sequences that is at least 70%, 75%, 80%, 85%, 90%, 91%, 92%, 93%, 94%, 95%, 96%, 97%, 98%, 99%, 99.5% or more identical to the nucleotide sequence set forth in any one of SEQ ID NOs:1 to 11, 29 to 30, 32 to 34, one or more nucleotide sequences that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence of any one of SEQ ID NOs:12 to 16, 31, one or more nucleotide sequences that is complementary to one of the aforementioned nucleotide sequences, one or more nucleotide sequences that specifically hybridize to one of the aforementioned nucleotide sequences under stringent hybridization conditions, and/or a functional fragment of one of the aforementioned nucleotide sequences; and producing a plant from the plant part.

In some embodiments, the present invention provides a method of producing a plant having increased yield, increased yield stability, and/or enhanced drought stress tolerance, the method comprising introducing, into a plant part, one or more nucleic acids comprising one or more of the nucleotide sequences set forth in any one of SEQ ID NOs:1 to 11, 29 to 30, 32 to 34, one or more nucleotide sequences that encodes a polypeptide comprising the amino acid sequence of any one of SEQ ID NOs:12 to 16, 31, one or more nucleotide sequences that is at least 70%, 75%, 80%, 85%, 90%, 91%, 92%, 93%, 94%, 95%, 96%, 97%, 98%, 99%, 99.5% or more identical to the nucleotide sequence set forth in any one of SEQ ID NOs:1 to 11, 29 to 30, 32 to 34, one or more nucleotide sequences that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence of any one of SEQ ID NOs:12 to 16, 31, one or more nucleotide sequences that is complementary to one of the aforementioned nucleotide sequences, one or more nucleotide sequences that specifically hybridize to one of the aforementioned nucleotide sequences under stringent hybridization conditions, and/or a functional fragment of one of the aforementioned nucleotide sequences; and producing a plant from the plant part.

In some embodiments, the present invention provides a method of producing a plant having increased yield, increased yield stability, and/or enhanced drought stress tolerance, the method comprising crossing a first parent plant and/or plant part with a second parent plant and/or plant part, wherein the first parent plant and/or plant part comprises within its genome one or more exogenous nucleic acids comprising one or more of the nucleotide sequences set forth in any one of SEQ ID NOs:1 to 11, 29 to 30, 32 to 34, one or more nucleotide sequences that encodes a polypeptide comprising the amino acid sequence of any one of SEQ ID NOs:12 to 16, 31, one or more nucleotide sequences that is at least 70%, 75%, 80%, 85%, 90%, 91%, 92%, 93%, 94%, 95%, 96%, 97%, 98%, 99%, 99.5% or more identical to the nucleotide sequence set forth in any one of SEQ ID NOs:1 to 11, 29 to 30, 32 to 34, one or more nucleotide sequences that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence of any one of SEQ ID NOs:12 to 16, 31, one or more nucleotide sequences that is complementary to one of the aforementioned nucleotide sequences, one or more nucleotide sequences that specifically hybridize to one of the aforementioned nucleotide sequences under stringent hybridization conditions, and/or a functional fragment of one of the aforementioned nucleotide sequences.

In some embodiments, the drought stress tolerance of a plant or plant part expressing a nucleic acid/protein of the present invention is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part. A “control plant and/or plant part” as used herein, including grammatical variations thereof, can include a plant and/or plant part of the same species (e.g., a parent plant) optionally grown under the same or substantially the same environmental conditions. For example, the drought stress tolerance of a plant and/or plant part expressing a nucleic acid encoding one or more SWEET 13 protein, SWEET 14 protein and/or SWEET 15 protein, each as described herein, may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part, optionally grown under the same (or substantially the same) drought stress conditions. Co-expression of one or more sugar (e.g., sucrose) 10 transporters and one or more T6PP proteins may likewise enhance the drought stress tolerance of a plant and/or plant part. In some embodiments, the drought stress tolerance of a plant and/or plant part expressing one or more SWEET 13 protein, SWEET 14 protein and/or SWEET 15 protein (e.g., one or more of SEQ ID NOs:12-16) as well as one or more T6PP proteins (e.g., one or more of SEQ ID NOs:21-24) may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300%, 350%, 400%, 450%, 500%, 550%, 600% or more as compared to a control plant and/or plant part, optionally grown under the same (or substantially the same) drought stress conditions.

Plants and plant parts expressing nucleic acids/proteins of the present invention may exhibit a variety of drought stress tolerant phenotypes, including, but not limited to, increased carbon (e.g., sucrose) concentration and/or availability, increased seed yield, increased harvest index, decreased embryo and/or kernel abortion, increased biomass, increased grain yield at standard moisture percentage (YGSMN), increased grain moisture at harvest (GMSTP), increased grain weight per plot (GWTPN), increased percent yield recovery (PYREC), decreased yield reduction (YRED), and/or decreased percent barren (PB) when grown under drought stress conditions. In some embodiments, one or more drought stress tolerant phenotypes is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300%, or more as compared to a control plant and/or plant part. In some embodiments, one or more drought stress tolerant phenotypes is decreased by at least about 5%, 10%, 15%, 20%, 25%, 30%, 35%, 40%, 45%, 50%, 55%, 60%, 65%, 75%, 80%, 85%, 90%, 95%, or more as compared to a control plant and/or plant part.

In some embodiments, the yield (e.g., seed yield, biomass, harvest index, GWTPN, PYREC and/or YGSMN) of a plant and/or plant part expressing a nucleic acid/protein of the present invention is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part. For example, the seed yield, biomass, and/or harvest index of a plant and/or plant part expressing one or more of SEQ ID NOs:12 to 16, 31 and optionally expressing one or more of SEQ IDs:21-24 may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part. In some embodiments, the yield of a plant and/or plant part expressing a nucleic acid/protein of the present invention may be increased when grown under drought stress conditions, as compared to a control plant and/or plant part grown under the same or substantially the same drought stress conditions.

Some embodiments include that the yield stability of a plant and/or plant part expressing a nucleic acid/protein of the present invention and grown under drought stress conditions is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part. For example, the yield stability of a plant and/or plant part under drought stress conditions that expresses one or more of SEQ ID NOs:12 to 16, 31 and optionally expresses one or more of SEQ IDs:21-24 may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part. In some embodiments, the yield stability of a plant and/or plant part expressing a nucleic acid/protein of the present invention may be increased under drought stress conditions.

In some embodiments, yield stability may refer to the ability of a plant and/or plant part expressing a nucleic acid/protein of the present invention to preserve the yield under drought stress conditions compared to a control plant and/or plant part under the same or substantially the same drought stress conditions. In some embodiments, an increase in yield stability may be determined by comparing the yield of plant and/or plant part expressing a nucleic acid/protein of the present invention obtained under both non-drought and drought stress conditions with the yield of a control plant and/or plant part obtained under both non-drought and drought stress conditions.

In some embodiments, the expression, stability and/or activity of one or more sugar (e.g., sucrose) transporters in a plant or plant part expressing a nucleic acid/protein of the present invention is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part. For example, the expression, stability and/or activity of one or more SWEET 13 proteins, SWEET 14 proteins, SWEET 15 proteins and/or T6PP proteins may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part.

In some embodiments, it may be preferable to target expression of nucleic acids of the present invention to different cellular localizations in the plant. In some cases, localization in the cytosol may be desirable, whereas in other cases, localization in some subcellular organelle may be preferred. Subcellular localization of transgene-encoded enzymes is undertaken using techniques well known in the art. Typically, a nucleotide sequence encoding a target peptide from a known organelle-targeted gene product is manipulated and fused upstream of the nucleotide sequence. Many such target sequences are known for the chloroplast and their functioning in heterologous constructions has been shown. The expression of the nucleotide sequences of the present invention may also be targeted to the endoplasmic reticulum or to the vacuoles of the host cells. Techniques to achieve this are well known in the art.

In some embodiments, it may be desirable to target proteins of the present invention to particular parts of a cell such as the chloroplast, the cell wall, the mitochondria, and the like. A nucleotide sequence encoding a signal peptide may be operably linked at the 5′- or 3′-terminus of a heterologous nucleotide sequence or nucleic acid molecule.

Various mechanisms for targeting gene products are known to exist in plants and the sequences controlling the functioning of these mechanisms have been characterized in some detail. For example, the targeting of gene products to the chloroplast is controlled by a signal sequence found at the amino terminal end of various proteins, which is cleaved during chloroplast import to yield the mature protein (see, e.g., Comai et al., J. BIOL. CHEM. 263:15104-15109 (1988). These signal sequences may be fused to heterologous gene products to effect the import of heterologous products into the chloroplast (see, e.g., van den Broeck et al., NATURE 313:358-363(1985)). DNA encoding appropriate signal sequences may be isolated from the 5′ end of the cDNAs encoding the RUBISCO protein, the CAB protein, the EPSP synthase enzyme, the GS2 protein and many other proteins that are known to be chloroplast localized.

The above-described targeting sequences may be utilized not only in conjunction with their endogenous promoters, but also in conjunction with heterologous promoters. Use of promoters that are heterologous to the targeting sequence not only provides the ability to target the sequence but also can provide an expression pattern that is different from that of the promoter from which the targeting signal is originally derived.

Signal peptides (and the targeting nucleotide sequences encoding them) are well known in the art and can be found in public databases such as the “Signal Peptide Website: An Information Platform for Signal Sequences and Signal Peptides.” (www.signalpeptide.de); the “Signal Peptide Database” (proline.bic.nus.edu.sg/spdb/index.html) (Choo et al., BMC BIOINFORMATICS 6:249 (2005)(available on www.biomedcentral.com/1471-2105/6/249/abstract); ChloroP (www.cbs.dtu.dk/services/ChloroP/; predicts the presence of chloroplast transit peptides (cTP) in protein sequences and the location of potential cTP cleavage sites); LipoP (www.cbs.dtu.dk/services/LipoP/; predicts lipoproteins and signal peptides in Gram negative bacteria); MITOPROT (ihg2.helmholtz-muenchen.de/ihg/mitoprot.html; predicts mitochondrial targeting sequences); PlasMit (gecco.org.chemie.uni-frankfurt.de/plasmit/index.html; predicts mitochondrial transit peptides in Plasmodium falciparum); Predotar (urgi.versailles.inra.fr/predotar/predotar.html; predicts mitochondrial and plastid targeting sequences); PTS1 (mendel.imp.ac.at/mendeljsp/sat/pts1/PTS1predictor.jsp; predicts peroxisomal targeting signal 1 containing proteins); SignalP (www.cbs.dtu.dk/services/SignalP/; predicts the presence and location of signal peptide cleavage sites in amino acid sequences from different organisms: Gram-positive prokaryotes, Gram-negative prokaryotes, and eukaryotes).

Thus, for example, to localize to a plastid, a transit peptide from plastidic Ferredoxin: NADP+ oxidoreductase (FNR) of spinach, which is disclosed in Jansen et al., CURRENT GENETICS 13:517-522 (1988), may be employed. In particular, the sequence ranging from the nucleotides −171 to 165 of the cDNA sequence disclosed therein may be used, which comprises the 5′ non-translated region as well as the sequence encoding the transit peptide. Another example of a transit peptide is that of the waxy protein of maize including the first 34 amino acid residues of the mature waxy protein (Klosgen et al. MOL. GEN. GENET. 217:155-161 (1989)). It is also possible to use this transit peptide without the first 34 amino acids of the mature protein. Furthermore, the signal peptides of the ribulose bisposphate carboxylase small subunit (Wolter et al. PROC. NATL. ACAD. SCI. USA 85:846-850 (1988); Nawrath et al. PROC. NATL. ACAD. SCI. USA 91:12760-12764 (1994)), of NADP malate dehydrogenase (Galiardo et al. PLANTA 197:324-332 (1995)), of glutathione reductase (Creissen et al. PLANT J. 8:167-175(1995)) and/or of the R1 protein (Lorberth et al. NATURE BIOTECHNOLOGY 16:473-477 (1998)) may be used.

The present invention also encompasses amplification primers (and pairs of amplification primers) useful for isolating, amplifying, and/or identifying SWEET 13 proteins, SWEET 14 proteins, SWEET 15 proteins and/or T6PP proteins.

The present invention extends to uses of nucleic acids, expression cassettes, vectors, bacteria, viruses, fungi, proteins, and/or amplification primers of the present invention, including, but not limited to, uses for increasing yield, uses for increasing yield stability under drought stress conditions, uses for enhancing drought stress tolerance in a plant and/or plant part, and/or uses for identifying, selecting and/or producing such a plant and/or plant part.

In some embodiments, the use comprises introducing a nucleic acid of the present invention into a plant cell, growing the transgenic plant cell into a transgenic plant and/or plant part, and, optionally, selecting the transgenic plant and/or plant part based upon increased yield, increased yield stability under drought stress conditions, and/or enhanced drought stress tolerance. Such uses may comprise transforming the plant cell with a transgenic bacterium/virus of the present invention.

In some embodiments, the use comprises culturing a transgenic bacterium/fungus comprising a nucleic acid of the present invention in/on a culture medium; isolating, from the culture medium, a protein encoded by the nucleic acid; and applying the protein to a plant and/or plant part.

In some embodiments, the use comprises infecting a plant and/or plant part with a transgenic virus comprising a nucleic acid of the present invention.

In some embodiments, the use comprises applying a protein of the present invention to a plant and/or plant part.

The present invention also provides nonnaturally occurring plants and plant parts having increased yield, increased yield stability under drought stress conditions, and/or enhanced drought stress tolerance.

A plant and/or plant part of the present invention may comprise any suitable exogenous nucleic acid(s). In some embodiments, the plant and/or plant part comprises at least one exogenous nucleic acid that encodes one or more proteins of the present invention and/or comprises, consists essentially of, or consists of one or more nucleic acids of the present invention.

Water deficit during the transition to reproductive development disrupts sucrose supply to developing ears and greatly impacts yield. Maize transgenic plants expressing a rice trehalose-6-phosphate phosphatase (T6PP) have demonstrated drought yield preservation. The T6PP transgenic plants consistently show increased levels of sucrose in leaf and floret tissue.

Molecular profiling experiments of T6PP maize transgenic plants have identified an increase in sucrose due to up-regulation of the transcripts of ZmSWEET13a, ZmSWEET14b and ZmSWEET15b transporter. Based on this observation, it is predicted that targeted over expression of these SWEET genes will generate an increase in the allocation of sucrose in the developing ear. Although not to be limited by theory, this change in sucrose allocation should have a positive impact on yield. Overexpression of SWEET13, SWEET14 and/or SWEET15, alone or in combination in specific tissues of a plant, such as maternal tissues of a developing ear, should provide a ready supply of carbon and energy during the critical stage of reproductive development. This increase in carbon and energy is predicted to help the plant cope with a decreased sugar supply when the plant is under an abiotic stress, such as a drought stress. In addition, an increase in SWEET13, SWEET14 and/or SWEET15 expression targeted to the leaf may increase phloem loading resulting in increased sucrose content in the stem and/or translocation to developing kernels.

In some embodiments, a plant and/or plant part comprises within its genome an exogenous nucleic acid that comprises, consists essentially of, or consists of:
(a) a nucleotide sequence set forth in any one of SEQ ID NOs:1 to 11, 29 to 30, 32 to 34;
(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs:1 to 11, 29 to 30, 32 to 34;
(c) a nucleotide sequence that encodes a polypeptide comprising the amino acid sequence set forth in any one of SEQ ID NOs:12 to 16, 31;
(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs:12 to 16, 31;
(e) a nucleotide sequence that is complementary to the nucleotide sequence of any one of (a) to (d) above;
(f) a nucleotide sequence that hybridizes to the nucleotide sequence of any one of (a) to (e) above under stringent hybridization conditions;
(g) a functional fragment of any one of (a) to (f) above, wherein the functional fragment encodes a sucrose transporter;
(h) a functional fragment of any one of (a) to (f) above, wherein the functional fragment encodes a polypeptide that comprises five alpha-helical transmembrane domains;
(i) a functional fragment of any one of (a) to (f) above, wherein the functional fragment encodes a polypeptide that comprises six alpha-helical transmembrane domains;
(j) a functional fragment of any one of (a) to (f) above, wherein the functional fragment encodes a polypeptide that comprises seven alpha-helical transmembrane domains, and any combination thereof.

In some embodiments, a plant and/or plant part may comprise two or more nucleotide sequences that encode the same or different SWEET proteins (e.g., the same or different SWEET 13, SWEET 14 and/or SWEET 15 proteins). The two or more nucleotide sequences may be operably linked to the same promoter, separate promoters, or any combination thereof. When separate promoters are used for the two or more nucleotides, the same or different promoters may be used. In some embodiments, a tissue-specific promoter sequence may be used, such as, for example, a flower-, seed-, endosperm-, embryo-, panicle-, and/or node-specific promoter sequence.

Some embodiments include that a plant and/or plant part comprises at least two nucleotide sequences that are each independently selected from the group consisting of:
(a) a nucleotide sequence set forth in any one of SEQ ID NOs:1 to 11, 29 to 30, 32 to 34;
(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs:1 to 11, 29 to 30, 32 to 34;
(c) a nucleotide sequence that encodes a polypeptide comprising the amino acid sequence set forth in any one of SEQ ID NOs:12 to 16, 31;
(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs:12 to 16, 31;
(e) a nucleotide sequence that is complementary to the nucleotide sequence of any one of (a) to (d) above;
(f) a nucleotide sequence that hybridizes to the nucleotide sequence of any one of (a) to (e) above under stringent hybridization conditions;
(g) a functional fragment of any one of (a) to (f) above, wherein the functional fragment encodes a sucrose transporter;
(h) a functional fragment of any one of (a) to (f) above, wherein the functional fragment encodes a polypeptide that comprises five alpha-helical transmembrane domains;
(i) a functional fragment of any one of (a) to (f) above, wherein the functional fragment encodes a polypeptide that comprises six alpha-helical transmembrane domains; and
(j) a functional fragment of any one of (a) to (f) above, wherein the functional fragment encodes a polypeptide that comprises seven alpha-helical transmembrane domains.

In some embodiments, a plant and/or plant part may comprise at least one nucleotide sequence that is at least 70% identical to the nucleotide sequence of any one of SEQ ID NOs:1 to 2 or 6 to 11 or that encodes a polypeptide comprising an amino acid sequence that is at least 70% identical to the amino acid sequence set forth in any one of SEQ ID NOs: 12 or 14 to 16, and at least one nucleotide sequence that is at least 70% identical to the nucleotide sequence of any one of SEQ ID NOs:3 to 5 or that encodes a polypeptide comprising an amino acid sequence that is at least 70% identical to the amino acid sequence set forth in SEQ ID NO:13.

Some embodiments include that a plant and/or plant part may comprise a nucleotide sequence that encodes a SWEET protein (e.g., a SWEET 13, SWEET 14 protein and/or SWEET 15) and a nucleotide sequence that encodes a T6PP protein. In some embodiments, the nucleotide sequence encoding a T6PP protein may be at least 70% identical to the nucleotide sequence of any one of SEQ ID NOs:17-20 and/or at least 70% identical to a nucleotide sequence that encodes a polypeptide comprising the amino acid sequence of any one of SEQ ID NOs:21 to 24. The nucleotide sequence encoding a T6PP protein may be operably linked to a promoter. The promoter for the nucleotide sequence encoding a T6PP protein may be operably linked to the same promoter and/or a separate promoter from a promoter operably linked to a nucleotide sequence encoding a SWEET protein (e.g., a SWEET 13, SWEET 14 protein and/or SWEET 15). When a separate promoter is used for the nucleotide sequence encoding a T6PP protein, the same promoter and/or a different promoter may be used as that for a nucleotide sequence encoding a SWEET protein.

A plant and/or plant part of the present invention may exhibit increased yield compared to a control plant and/or plant part. In some embodiments, a plant and/or plant part of the present invention may exhibit increased yield under non-abiotic stress conditions and/or abiotic stress conditions (e.g., drought stress conditions). In some embodiments, the yield (e.g., seed yield, biomass, harvest index, GWTPN, PYREC and/or YGSMN) of the plant and/or plant part is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part. For example, the seed yield, harvest index, and/or biomass of the plant and/or plant part may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part.

Some embodiments include that a plant and/or plant part of the present invention may exhibit increased yield stability under drought stress conditions compared to a control plant and/or plant part. In some embodiments, yield stability of the plant and/or plant part may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more.

In some embodiments, a plant and/or plant part of the present invention may exhibit enhanced drought stress tolerance compared to a control plant and/or plant part. The plant and/or plant part may exhibit a variety of drought stress tolerant phenotypes, including, but not limited to, increased carbon (e.g., sucrose) concentration and/or availability, decreased embryo and/or kernel abortion, increased survival rate, and/or increased yield (e.g., increased biomass, increased seed yield, increased harvest index, increased GSC, increased YGSMN, increased GMSTP, increased GWTPN, increased PYREC, decreased YRED, and/or decreased PB when grown under drought stress conditions. In some embodiments, one or more drought stress tolerant phenotypes is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300%, or more as compared to a control plant and/or plant part. In some embodiments, one or more drought stress tolerant phenotypes is decreased by at least about 5%, 10%, 15%, 20%, 25%, 30%, 35%, 40%, 45%, 50%, 55%, 60%, 65%, 75%, 80%, 85%, 90%, 95%, or 100% as compared to a control plant and/or plant part.

In some embodiments, the expression, stability and/or activity of one or more sucrose 10 transporters (e.g., one or more SWEET proteins) and/or one or more T6PP proteins in a plant and/or plant part is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part. For example, the expression, stability and/or activity of one or more SWEET 13 proteins, one or more SWEET 14 proteins, one or SWEET 15 proteins and/or one or more T6PP proteins may be increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part. In some embodiments, the control plant and/or plant part may not comprise the exogenous nucleic acid(s) of the present invention (e.g., one or more SWEET 13 proteins, SWEET 14 proteins, one or more SWEET 15 proteins and/or T6PP proteins), but may comprise endogenous SWEET 13 proteins, SWEET 14 proteins, SWEET 15 proteins and/or T6PP proteins.

In some embodiments, the drought stress tolerance of a plant and/or plant part of the present invention is increased by at least about 5%, 10%, 15%, 20%, 25%, 30%, 35%, 40%, 45%, 50%, 55%, 60%, 65%, 75%, 80%, 85%, 90%, 95%, 100%, 125%, 150%, 175%, 200%, 250%, 300% or more as compared to a control plant and/or plant part.

A plant and/or plant part of the present invention may be of any suitable plant type, including, but not limited to, plants belonging to the superfamily Viridiplantae. In some embodiments the plant or plant part is a fodder crop, a food crop, an ornamental plant, a tree or a shrub. A plant and/or plant part of the present invention may be produced using any suitable method, including, but not limited to, methods of the present invention.

The present invention also encompasses methods of increasing yield, increasing yield stability under drought stress conditions, and/or enhancing drought stress tolerance in a plant and/or plant part.

Increasing yield, increasing yield stability under drought stress conditions, and/or enhancing drought stress tolerance may be carried out by increasing the expression, stability and/or activity of one or more SWEET 13 proteins, one or more SWEET 14 proteins, one or more SWEET 15 proteins and/or one or more T6PP proteins. Thus, methods of increasing yield, increasing yield stability under drought stress conditions, and/or enhancing drought stress tolerance in a plant and/or plant part may comprise, consist essentially of, or consist of increasing the expression, stability and/or activity of one or more SWEET 13 proteins, one or more SWEET 14 proteins, one or more SWEET 15 proteins and/or one or more T6PP proteins in the plant or plant part. In some embodiments, one or more SWEET 13 proteins, one or more SWEET 14 proteins, one or more SWEET 15 proteins and/or one or more T6PP proteins are overexpressed. Overexpression may be determined by comparing the expression of the one or more SWEET 13 proteins, one or more SWEET 14 proteins, one or more SWEET 15 proteins and/or one or more T6PP proteins to the expression of the same SWEET 13 protein, SWEET 14 protein, SWEET 15 protein and T6PP protein in a control plant and/or plant part.

Thus, in some embodiments, yield may be increased, yield stability under drought stress conditions may be increased, and/or drought stress tolerance may be enhanced by introducing/expressing an exogenous nucleic acid comprising:
(a) a nucleotide sequence set forth in any one of SEQ ID NOs:1 to 11, 29 to 30, 32 to 34;
(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs:1 to 11, 29 to 30, 32 to 34;
(c) a nucleotide sequence that encodes a polypeptide comprising the amino acid sequence set forth in any one of SEQ ID NOs:12 to 16, 31;
(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs:12 to 16, 31;
(e) a nucleotide sequence that is complementary to the nucleotide sequence of any one of (a) to (d) above;
(f) a nucleotide sequence that hybridizes to the nucleotide sequence of any one of (a) to (e) above under stringent hybridization conditions;
(g) a functional fragment of any one of (a) to (f) above, wherein the functional fragment encodes a sucrose transporter;
(h) a functional fragment of any one of (a) to (f) above, wherein the functional fragment encodes a polypeptide that comprises five alpha-helical transmembrane domains;
(i) a functional fragment of any one of (a) to (f) above, wherein the functional fragment encodes a polypeptide that comprises six alpha-helical transmembrane domains;
(j) a functional fragment of any one of (a) to (f) above, wherein the functional fragment encodes a polypeptide that comprises seven alpha-helical transmembrane domains, and any combination thereof.

The present invention also encompasses methods of identifying, selecting and/or producing a plant and/or plant part having increased yield, increased yield stability under drought stress conditions, and/or enhanced drought stress tolerance.

Methods of identifying a plant and/or plant part having increased yield, increased yield stability under drought stress conditions, and/or enhanced drought stress tolerance may comprise, consist essentially of, or consist of detecting, in the plant and/or plant part, a nucleic acid (e.g., an exogenous nucleic acid) comprising:
(a) a nucleotide sequence set forth in any one of SEQ ID NOs:1 to 11, 29 to 30, 32 to 34;
(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs:1 to 11, 29 to 30, 32 to 34;
(c) a nucleotide sequence that encodes a polypeptide comprising the amino acid sequence set forth in any one of SEQ ID NOs:12 to 16, 31;
(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs:12 to 16, 31;
(e) a nucleotide sequence that is complementary to the nucleotide sequence of any one of (a) to (d) above;
(f) a nucleotide sequence that hybridizes to the nucleotide sequence of any one of (a) to (e) above under stringent hybridization conditions;
(g) a functional fragment of any one of (a) to (f) above, wherein the functional fragment encodes a sucrose transporter;
(h) a functional fragment of any one of (a) to (f) above, wherein the functional fragment encodes a polypeptide that comprises five alpha-helical transmembrane domains;
(i) a functional fragment of any one of (a) to (f) above, wherein the functional fragment encodes a polypeptide that comprises six alpha-helical transmembrane domains;
(j) a functional fragment of any one of (a) to (f) above, wherein the functional fragment encodes a polypeptide that comprises seven alpha-helical transmembrane domains, and any combination thereof.

Methods of producing a plant or plant part having increased yield, increased yield stability under drought stress conditions, and/or enhanced drought stress tolerance may comprise, consist essentially of, or consist of:
(a) detecting, in a plant part, the presence of an exogenous nucleic acid encoding one or more SWEET 13 proteins, one or more SWEET 14 proteins, one or more SWEET 15 proteins and/or one or more T6PP proteins, and producing a plant from the plant part;
(b) introducing, into a plant part, an exogenous nucleic acid encoding one or more SWEET 13 proteins, one or more SWEET 14 proteins, one or more SWEET 15 proteins and/or one or more T6PP proteins, and growing the plant part into a plant; such methods may further comprise detecting the exogenous nucleic acid in the plant part and/or in the plant produced from the plant part;
(c) introducing, into a plant part, an exogenous nucleic acid encoding one or more SWEET 13 proteins, one or more SWEET 14 proteins, one or more SWEET 15 and/or one or more T6PP proteins, detecting the presence of the exogenous nucleic acid in the plant part, and growing the plant part into a plant;
(d) crossing a first parent plant or plant part with a second parent plant or plant part, wherein the first parent plant or plant part comprises within its genome a nucleic acid (e.g., an exogenous nucleic acid) encoding one or more SWEET 13 proteins, one or more SWEET 14 proteins, one or more SWEET 15 proteins and/or one or more T6PP proteins; and/or
(e) introgressing an exogenous nucleic acid encoding one or more SWEET 13 proteins, one or more SWEET 14 proteins, one or more SWEET 15 proteins and/or one or more T6PP proteins into a plant or plant part lacking the exogenous nucleic acid.

In some embodiments, a method of producing a plant or plant part having increased yield, increased yield stability under drought stress conditions, and/or enhanced drought stress tolerance comprises, consists essentially of or consists of detecting, in a plant and/or plant part, the presence of a nucleic acid (e.g., an exogenous nucleic acid) comprising, consisting essentially of or consisting of:
(a) a nucleotide sequence set forth in any one of SEQ ID NOs:1 to 11, 29 to 30, 32 to 34;
(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs:1 to 11, 29 to 30, 32 to 34;
(c) a nucleotide sequence that encodes a polypeptide comprising the amino acid sequence set forth in any one of SEQ ID NOs:12 to 16, 31;
(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs:12 to 16, 31;
(e) a nucleotide sequence that is complementary to the nucleotide sequence of any one of (a) to (d) above;
(f) a nucleotide sequence that hybridizes to the nucleotide sequence of any one of (a) to (e) above under stringent hybridization conditions;
(g) a functional fragment of any one of (a) to (f) above, wherein the functional fragment encodes a sucrose transporter;
(h) a functional fragment of any one of (a) to (f) above, wherein the functional fragment encodes a polypeptide that comprises five alpha-helical transmembrane domains;
(i) a functional fragment of any one of (a) to (f) above, wherein the functional fragment encodes a polypeptide that comprises six alpha-helical transmembrane domains; and/or
(j) a functional fragment of any one of (a) to (f) above, wherein the functional fragment encodes a polypeptide that comprises seven alpha-helical transmembrane domains, and producing a plant from the plant or plant part, thereby producing a plant having increased yield, increased yield stability under drought stress conditions, and/or enhanced drought stress tolerance as compared to a control plant.

In some embodiments, a method of producing a plant or plant part having increased yield, increased yield stability under drought stress conditions, and/or enhanced drought stress tolerance comprises, consists essentially of, or consists of introducing, into a plant and/or plant part, an exogenous nucleic acid comprising, consisting essentially of or consisting of:
(a) a nucleotide sequence set forth in any one of SEQ ID NOs:1 to 11, 29 to 30, 32 to 34;
(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs:1 to 11, 29 to 30, 32 to 34;
(c) a nucleotide sequence that encodes a polypeptide comprising the amino acid sequence set forth in any one of SEQ ID NOs:12 to 16, 31;
(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs:12 to 16, 31;
(e) a nucleotide sequence that is complementary to the nucleotide sequence of any one of (a) to (d) above;
(f) a nucleotide sequence that hybridizes to the nucleotide sequence of any one of (a) to (e) above under stringent hybridization conditions;
(g) a functional fragment of any one of (a) to (f) above, wherein the functional fragment encodes a sucrose transporter;
(h) a functional fragment of any one of (a) to (f) above, wherein the functional fragment encodes a polypeptide that comprises five alpha-helical transmembrane domains;
(i) a functional fragment of any one of (a) to (f) above, wherein the functional fragment encodes a polypeptide that comprises six alpha-helical transmembrane domains; and/or
(j) a functional fragment of any one of (a) to (f) above, wherein the functional fragment encodes a polypeptide that comprises seven alpha-helical transmembrane domains, and producing a plant from the plant or plant part, thereby producing a plant having increased yield, increased yield stability under drought stress conditions, and/or enhanced drought stress tolerance as compared to a control plant.

In some embodiments, a method of producing a plant having increased yield, increased yield stability under drought stress conditions, and/or enhanced drought stress tolerance comprises, consists essentially of or consists of crossing a first parent plant or plant part with a second parent plant or plant part, wherein the first parent plant or plant part comprises within its genome a nucleic acid (e.g., an exogenous nucleic acid) comprising, consisting essentially of or consisting of:
(a) a nucleotide sequence set forth in any one of SEQ ID NOs:1 to 11, 29 to 30, 32 to 34;
(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs:1 to 11, 29 to 30, 32 to 34;
(c) a nucleotide sequence that encodes a polypeptide comprising the amino acid sequence set forth in any one of SEQ ID NOs:12 to 16, 31;
(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs:12 to 16, 31;
(e) a nucleotide sequence that is complementary to the nucleotide sequence of any one of (a) to (d) above;
(f) a nucleotide sequence that hybridizes to the nucleotide sequence of any one of (a) to (e) above under stringent hybridization conditions;
(g) a functional fragment of any one of (a) to (f) above, wherein the functional fragment encodes a sucrose transporter;
(h) a functional fragment of any one of (a) to (f) above, wherein the functional fragment encodes a polypeptide that comprises five alpha-helical transmembrane domains;
(i) a functional fragment of any one of (a) to (f) above, wherein the functional fragment encodes a polypeptide that comprises six alpha-helical transmembrane domains; and/or
(j) a functional fragment of any one of (a) to (f) above, wherein the functional fragment encodes a polypeptide that comprises seven alpha-helical transmembrane domains, thereby producing a progeny generation that comprises at least one plant that comprises the nucleic acid (or a functional fragment thereof) and that exhibits increased yield, increased yield stability under drought stress conditions, and/or enhanced drought stress tolerance as compared to a control plant. Such methods may further comprise selecting a progeny plant and/or plant part that comprises the nucleic acid (or a functional fragment thereof) within its genome and that exhibits increased yield, increased yield stability under drought stress conditions, and/or enhanced drought stress tolerance as compared to a control plant. Such selections may be made based upon the detection of the nucleic acid (or a functional fragment thereof) in the plant and/or plant part and/or the increased yield, increased yield stability under drought stress conditions, and/or enhanced drought stress tolerance of the progeny plant or part.

In some embodiments, a method of producing a plant having increased yield, increased yield stability under drought stress conditions, and/or enhanced drought stress tolerance comprises, consists essentially of or consists of crossing a first plant or plant part that comprises a nucleic acid (e.g., an exogenous nucleic acid) encoding one or more SWEET 13 proteins, one or more SWEET 14 proteins, one or more SWEET 15 proteins and/or one or more T6PP proteins with a second plant or plant part that lacks the nucleic acid and repeatedly backcrossing progeny plants comprising the nucleic acid (or a functional fragment thereof) with the second plant or plant part to produce an introgressed plant or plant part that comprises the nucleic acid (or a functional fragment thereof) and that exhibits increased yield, increased yield stability under drought stress conditions, and/or enhanced drought stress tolerance as compared to a control plant. In some embodiments, the method further comprises selecting the introgressed plant or plant part based upon the presence of the nucleic acid (or a functional fragment thereof) in the plant and/or plant part and/or its increased yield, increased yield stability under drought stress conditions, and/or enhanced drought stress tolerance. In some embodiments, the method further comprises selecting the introgressed plant or plant part (for inclusion in a breeding program, for example).

In some embodiments, a method of producing a plant and/or plant part having increased yield, increased yield stability under drought stress conditions, and/or enhanced drought stress tolerance comprises, consists essentially of or consists of crossing a first plant or plant part that comprises a nucleic acid (e.g., an exogenous nucleic acid) with a second plant or plant part that lacks the nucleic acid and repeatedly backcrossing progeny plants comprising the nucleic acid (or a functional fragment thereof) with the second plant or plant part to produce an introgressed plant or plant part that comprises the nucleic acid (or a functional fragment thereof) and that exhibits increased yield, increased yield stability under drought stress conditions, and/or enhanced drought stress tolerance as compared to a control plant, wherein the exogenous nucleic acid comprises, consists essentially of or consists of:
(a) a nucleotide sequence set forth in any one of SEQ ID NOs:1 to 11, 29 to 30, 32 to 34;
(b) a nucleotide sequence that is at least 70%, 75%, 80%, 85%, 90%, 91%, 92%, 93%, 94%, 95%, 96%, 97%, 98%, 99%, 99.5% or more identical to the nucleotide sequence of any one of SEQ ID NOs:1 to 11, 29 to 30, 32 to 34;
(c) a nucleotide sequence that encodes a polypeptide comprising the amino acid sequence set forth in any one of SEQ ID NOs:12 to 16, 31;
(d) a nucleotide sequence that encodes a polypeptide comprising an amino acid sequence that is at least 70%, 75%, 80%, 85%, 90%, 91%, 92%, 93%, 94%, 95%, 96%, 97%, 98%, 99%, 99.5% or more identical to the amino acid sequence set forth in any one of SEQ ID NOs:12 to 16, 31;
(e) a nucleotide sequence that is complementary to the nucleotide sequence of any one of (a) to (d) above;
(f) a nucleotide sequence that hybridizes to the nucleotide sequence of any one of (a) to (e) above under stringent hybridization conditions;
(g) a functional fragment of any one of (a) to (f) above, wherein the functional fragment encodes a sucrose transporter;
(h) a functional fragment of any one of (a) to (f) above, wherein the functional fragment encodes a polypeptide that comprises five alpha-helical transmembrane domains;
(i) a functional fragment of any one of (a) to (f) above, wherein the functional fragment encodes a polypeptide that comprises six alpha-helical transmembrane domains; and/or
(j) a functional fragment of any one of (a) to (f) above, wherein the functional fragment encodes a polypeptide that comprises seven alpha-helical transmembrane domains.

In some embodiments, the method further comprises selecting the introgressed plant or plant part based upon the presence of the nucleic acid (or a functional fragment thereof) and/or its increased yield, increased yield stability under drought stress conditions, and/or enhanced drought stress tolerance. In some embodiments, the method further comprises selecting the introgressed plant or plant part (for inclusion in a breeding program, for example).

Any suitable nucleic acid may be detected in or introduced into a plant and/or plant part, including, but not limited to, any nucleic acid of the present invention. In some embodiments, the nucleic acid detected in or introduced into the plant or plant part is a nucleic acid encoding one or more SWEET 13 proteins, one or more SWEET 14 proteins, one or more SWEET 15 proteins and/or one or more T6PP proteins.

Exogenous nucleic acids may be introduced into a plant and/or plant part via any suitable method, including, but not limited to, microparticle bombardment, liposome-mediated transfection, receptor-mediated delivery, bacteria-mediated delivery (e.g., Agrobacterium-mediated transformation and/or whiskers-mediated transformation). In some embodiments, the exogenous nucleic acid is introduced into a plant part by crossing a first plant or plant part comprising the exogenous nucleic acid with a second plant or plant part that lacks the exogenous nucleic acid.

“Introducing,” in the context of a nucleotide sequence of interest (e.g., a nucleotide sequence encoding a synthetic miRNA precursor molecule of the invention), means presenting the nucleotide sequence of interest to the plant, plant part, and/or plant cell in such a manner that the nucleotide sequence gains access to the interior of a cell. Where more than one nucleotide sequence is to be introduced these nucleotide sequences can be assembled as part of a single polynucleotide or nucleic acid construct, or as separate polynucleotide or nucleic acid constructs, and can be located on the same or different transformation vectors. Accordingly, these polynucleotides can be introduced into plant cells in a single transformation event, in separate transformation events, or, e.g., as part of a breeding protocol. Thus, for example, “introducing” can encompass transformation of an ancestor plant with a nucleotide sequence of interest followed by conventional breeding process to produce progeny comprising said nucleotide sequence of interest.

Transformation of a cell may be stable or transient. Thus, in some embodiments, a plant cell of the invention is stably transformed with a nucleotide sequence encoding a synthetic miRNA precursor molecule of the invention. In other embodiments, a plant of the invention is transiently transformed with a nucleotide sequence encoding a synthetic miRNA precursor molecule of the invention.

“Transient transformation” in the context of a polynucleotide means that a polynucleotide is introduced into the cell and does not integrate into the genome of the cell.

“Stable transformation” or “stably transformed,” “stably introducing,” or “stably introduced” as used herein means that a nucleic acid is introduced into a cell and integrates into the genome of the cell. As such, the integrated nucleic acid is capable of being inherited by the progeny thereof, more particularly, by the progeny of multiple successive generations. “Genome” as used herein also includes the nuclear and the plastid genome, and therefore includes integration of the nucleic acid into, for example, the chloroplast genome. Stable transformation as used herein can also refer to a transgene that is maintained extrachromasomally, for example, as a minichromosome.

Transient transformation may be detected by, for example, an enzyme-linked immunosorbent assay (ELISA) or Western blot, which can detect the presence of a peptide or polypeptide encoded by one or more transgene introduced into an organism. Stable transformation of a cell can be detected by, for example, a Southern blot hybridization assay of genomic DNA of the cell with nucleic acid sequences which specifically hybridize with a nucleotide sequence of a transgene introduced into an organism (e.g., a plant). Stable transformation of a cell can be detected by, for example, a Northern blot hybridization assay of RNA of the cell with nucleic acid sequences which specifically hybridize with a nucleotide sequence of a transgene introduced into a plant or other organism. Stable transformation of a cell can also be detected by, e.g., a polymerase chain reaction (PCR) or other amplification reactions as are well known in the art, employing specific primer sequences that hybridize with target sequence(s) of a transgene, resulting in amplification of the transgene sequence, which can be detected according to standard methods Transformation can also be detected by direct sequencing and/or hybridization protocols well known in the art.

Methods of introducing a nucleic acid into a plant can also comprise in vivo modification of nucleic acids, methods for which are known in the art. For example, in vivo modification can be used to insert a nucleic acid comprising, e.g., a promoter sequence into the plant genome. In a further non-limiting example, in vivo modification can be used to modify the endogenous nucleic acid itself and/or a endogenous transcription and/or translation factor associated with the endogenous nucleic acid, such that the transcription and/or translation of said endogenous nucleic acid is altered, thereby altering the expression said endogenous nucleic acid and/or in the case of nucleic acids encoding polypeptides, the production of said polypeptide.

Exemplary methods of in vivo modification include zinc finger nuclease, CRISPR-Cas, TALEN, TILLING (Targeted Induced Local Lesions IN Genomes) and/or engineered meganuclease technology.

For example, suitable methods for in vivo modification include the techniques described in Urnov et al. Nature Reviews 11:636-646 (2010)); Gao et. al., Plant J. 61, 176 (2010); Li et al., Nucleic Acids Res. 39, 359 (2011); Miller et al. 29, 143-148 (2011); Christian et al. Genetics 186, 757-761 (2010)); Jiang et al. Nat. Biotechnol. 31, 233-239 (2013)); U.S. Pat. Nos. 7,897,372 and 8,021,867; U.S. Patent Publication No. 2011/0145940 and in International Patent Publication Nos. WO 2009/114321, WO 2009/134714 and WO 2010/079430; U.S. Pat. Nos. 8,795,965 and 8,771,945 For example, one or more transcription affector-like nucleases (TALEN) and/or one or more meganucleases may be used to incorporate an isolated nucleic acid comprising a promoter sequence of the invention into the plant genome. In representative embodiments, the method comprises cleaving the plant genome at a target site with a TALEN and/or a meganuclease and providing a nucleic acid that is homologous to at least a portion of the target site and further comprises a promoter sequence of the invention (optionally in operable association with a heterologous nucleotide sequence of interest), such that homologous recombination occurs and results in the insertion of the promoter sequence of the invention into the genome. Alternatively, in some embodiments, a CRISPR-Cas system can be used to specifically edit the plant genome so as to alter the expression of endogenous nucleic acids described herein. In some embodiments, a genetic modification may also be introduced using the technique of TILLING, which combines high-density mutagenesis with high-throughput screening methods. Methods for TILLING are well known in the art (McCallum, Nature Biotechnol. 18, 455-457, 2000, Stemple, Nature Rev. Genet. 5, 145-150, 2004).

As would be understood by the skilled artisan, the polynucleotides of the invention can be modified in vivo using the above described methods as well as any other method of in vivo modification known or later developed.

Nucleic acids encoding SWEET proteins (e.g., SWEET 13 proteins, SWEET 14 proteins and/or SWEET 15) and/or T6PP proteins may be detected using any suitable method, including, but not limited to, DNA sequencing, mass spectrometry and capillary electrophoresis. In some embodiments, the nucleic acid (or an informative fragment thereof) is detected in one or more amplification products from a nucleic acid sample from the plant or plant part. In some such embodiments, the amplification product(s) comprise(s) the nucleotide sequence of any one of SEQ ID NOs:1-11, 29-30, the reverse complement thereof, an informative fragment thereof, or an informative fragment of the reverse complement thereof.

Nucleic acids encoding SWEET 13 proteins, SWEET 14 proteins, SWEET 15 proteins and/or T6PP proteins may be detected using any suitable probe. In some embodiments, the nucleic acid (or an informative fragment thereof) is detected using a probe comprising the nucleotide sequence of any one of SEQ ID NOs:1-11, the reverse complement thereof, an informative fragment thereof, or an informative fragment of the reverse complement thereof. In some embodiments, the probe comprises one or more detectable moieties, such as digoxigenin, fluorescein, acridine-ester, biotin, alkaline phosphatase, horseradish peroxidase, β-glucuronidase, β-galactosidase, luciferase, ferritin or a radioactive isotope.

Methods of the present invention may be used to identify, select and/or produce plants and/or plant parts that exhibit increased yield, increased yield stability under drought stress conditions, and/or enhanced drought stress tolerance.

Methods of the present invention may be used to identify, select, produce and/or protect plants and/or plant parts of any suitable plant type, including, but not limited to, plants belonging to the superfamily Viridiplantae. In some embodiments the plant or plant part is a fodder crop, a food crop, an ornamental plant, a tree or a shrub.

A plant and/or plant part of the present invention and/or suitable for use with the present invention may be of any plant type, including, but not limited to, plants belonging to the superfamily Viridiplantae and thus includes spermatophytes (e.g., angiosperms and gymnosperms) and embryophytes (e.g., bryophytes, ferns and fern allies). In some embodiments, a plant or plant part useful with this invention includes any monocot and/or any dicot plant or plant part. In some embodiments the plant or plant part is a fodder crop, a food crop, an ornamental plant, a tree or a shrub. For example, in some embodiments, the plant or plant part is a variety of Acer spp., Actinidia spp., Abelmoschus spp., Agropyron spp., Allium spp., Amaranthus spp., Ananas comosus, Annona spp., Apium graveolens, Arachis spp, Artocarpus spp., Asparagus officinalis, Avena spp. (e.g. Avena sativa, Avena fatua, Avena byzantina, Avena fatua var. sativa, Avena hybrida), Averrhoa carambola, Benincasa hispida, Bertholletia excelsea, Beta vulgaris, Brassica spp. (e.g. Brassica napus, Brassica rapa ssp. [canola, oilseed rape, turnip rape]), Cadaba farinosa, Camellia sinensis, Canna indica, Capsicum spp., Carex elata, Carica papaya, Carissa macrocarpa, Carya spp., Carthamus tinctorius, Castanea spp., Cichorium endivia, Cinnamomum spp., Citrullus lanatus, Citrus spp., Cocos spp., Coffea spp., Colocasia esculenta, Cola spp., Coriandrum sativum, Corylus spp., Crataegus spp., Crocus sativus, Cucurbita spp., Cucumis spp., Cynara spp., Daucus carota, Desmodium spp., Dimocarpus longan, Dioscorea spp., Diospyros spp., Echinochloa spp., Elaeis (e.g. Elaeis guineensis, Elaeis oleifera), Eleusine coracana, Eriobotrya japonica, Eugenia uniflora, Fagopyrum spp., Fagus spp., Ficus carica, Fortunella spp., Fragaria spp., Ginkgo biloba, Glycine spp. (e.g. Glycine max, Soja hispida or Soja max), Gossypium hirsutum, Helianthus spp. (e.g. Helianthus annuus), Hemerocallis fulva, Hibiscus spp., Hordeum spp. (e.g. Hordeum vulgare), Ipomoea batatas, Juglans spp., Lactuca sativa, Lathyrus spp., Lens culinaris, Linum usitatissimum, Litchi chinensis, Lotus spp., Luffa acutangula, Lupinus spp., Luzula sylvatica, Lycopersicon spp. (e.g. Lycopersicon esculentum, Lycopersicon lycopersicum, Lycopersicon pyriforme), Macrotyloma spp., Malus spp., Malpighia emarginata, Mammea americana, Mangifera indica, Manihot spp., Manilkara zapota, Medicago sativa, Melilotus spp., Mentha spp., Miscanthus spp., Momordica spp., Morus nigra, Musa spp., Nicotiana spp., Olea spp., Opuntia spp., Ornithopus spp., Oryza spp. (e.g. Oryza sativa, Oryza latifolia), Panicum miliaceum, Passiflora edulis, Pastinaca sativa, Persea spp., Petroselinum crispum, Phaseolus spp., Phoenix spp., Physalis spp., Pinus spp., Pistacia vera, Pisum spp., Poa spp., Populus spp., Prosopis spp., Prunus spp., Psidium spp., Punica granatum, Pyrus communis, Quercus spp., Raphanus sativus, Rheum rhabarbarum, Ribes spp., Ricinus communis, Rubus spp., Saccharum spp., Sambucus spp., Secale cereale, Sesamum spp., Sinapis sp., Solanum spp. (e.g. Solanum tuberosum, Solanum integrifolium or Solanum lycopersicum), Sorghum bicolor, Spinacia spp., Syzygium spp., Tagetes spp., Tamarindus indica, Theobroma cacao, Trifolium spp., Triticosecale rimpaui, Triticum spp. (e.g. Triticum aestivum, Triticum durum, Triticum turgidum, Triticum hybernum, Triticum macha, Triticum sativum or Triticum vulgare), Tropaeolum minus, Tropaeolum majus, Vaccinium spp., Vicia spp., Vigna spp., Viola odorata, Vitis spp., Zea mays, Zizania palustris or Ziziphus spp., amongst others.

In some embodiments, the plant and/or plant part is a rice, maize, wheat, barley, sorghum, millet, oat, triticale, rye, buckwheat, fonio, quinoa, sugar cane, bamboo, banana, ginger, onion, lily, daffodil, iris, amaryllis, orchid, canna, bluebell, tulip, garlic, secale, einkorn, spelt, emmer, durum, kamut, grass (e.g., gramma grass), teff, milo, flax, Tripsacum sp., or teosinte plant or plant part. In some embodiments, the plant or plant part is a blackberry, raspberry, strawberry, barberry, bearberry, blueberry, coffee berry, cranberry, crowberry, currant, elderberry, gooseberry, goji berry, honeyberry, lemon, lime, lingonberry, mangosteen, orange, pepper, persimmon, pomegranate, prune, cotton, clover, acai, plum, peach, nectarin, cherry, guava, almond, pecan, walnut, apple, amaranth, sweet pea, pear, potato, soybean, sugar beet, sunflower, sweet potato, tamarind, tea, tobacco or tomato plant or plant part.

The present invention extends to products harvested from plants and/or plant parts produced according to methods of the present invention. A harvested product can be a whole plant or any plant part, as described herein, wherein said harvested product comprises a recombinant nucleic acid molecule/nucleotide sequence of the invention. Thus, in some embodiments, non-limiting examples of a harvested product include a seed, a fruit, a flower or part thereof (e.g., an anther, a stigma, and the like), a leaf, a stem, and the like. In other embodiments, a post-harvested product includes, but is not limited to, a flour, meal, oil, starch, cereal, and the like produced from a harvested seed of the invention, wherein said seed comprises in its genome a recombinant nucleic acid molecule/nucleotide sequence of the invention.

Some embodiments include that the harvested product is a plant part capable of producing a plant and/or plant part that expresses one or more nonnaturally occurring proteins of the present invention. In some embodiments, the harvested product is a plant part capable of producing a plant and/or plant part that expresses one or more nonnaturally occurring SWEET 13 proteins, one or more nonnaturally occurring SWEET 14 proteins, one or more nonnaturally occurring SWEET 15 proteins and/or one or more nonnaturally occurring T6PP proteins. In some embodiments, the harvested product is a plant part capable of producing a plant and/or plant part that exhibits increased yield, increased yield stability (such as, for example, under drought conditions), and/or enhanced drought stress tolerance. In some embodiments, the harvested product is a plant part capable of producing a plant and/or plant part that exhibits increased carbon (e.g., sucrose) concentration and/or availability, decreased embryo and/or kernel abortion, increased survival rate, and/or increased yield (e.g., increased seed yield, increased harvest index, increased biomass, increased GSC, increased YGSMN, increased GMSTP, increased GWTPN, increased PYREC, decreased YRED, and/or decreased PB, optionally when grown under drought stress conditions.

The present invention also extends to products harvested from plants produced according to methods of the present invention, including, but not limited to, dry pellets and powders, oils, fats, fatty acids, starches and proteins.

In some embodiments, the invention further provides a plant crop comprising a plurality of transgenic plants of the invention planted together in, for example, an agricultural field, a golf course, a residential lawn, a road side, an athletic field, and/or a recreational field.

In some embodiments, a method of increasing the yield, increasing the yield stability under drought stress conditions, and/or enhancing the drought stress tolerance of a plant crop is provided, the method comprising cultivating a plurality of plants of the invention as the plant crop, wherein the plurality of plants of said plant crop have increased yield, increased yield stability under drought stress conditions, and/or enhanced drought stress tolerance, thereby increasing the yield, increasing the yield stability under drought stress conditions, and/or enhancing the drought stress tolerance of said plant crop as compared to a control plant crop, wherein the control plant crop is produced from a plurality of plants lacking said recombinant nucleic acid molecule grown under the same environmental conditions. In some particular embodiments of the invention, the plant crop can be a maize crop, a rice crop, or a wheat crop.

The invention will now be described with reference to the following examples. It should be appreciated that these examples are not intended to limit the scope of the claims to the invention, but are rather intended to be exemplary of certain embodiments. Any variations in the exemplified methods that occur to the skilled artisan are intended to fall within the scope of the invention.

### Examples

The following examples are not intended to be a detailed catalog of all the different ways in which the present invention may be implemented or of all the features that may be added to the present invention. Persons skilled in the art will appreciate that numerous variations and additions to the various embodiments may be made without departing from the present invention. Hence, the following descriptions are intended to illustrate some particular embodiments of the invention, and not to exhaustively specify all permutations, combinations and variations thereof.

### Example 1—Identification of Maize SWEET Genes Involved in Abiotic Stress

Transgenic maize plants expressing a rice trehalose-6-phosphate phosphatase (T6PP) polynucleotide under the control of an OsMADS6 promoter and showing significant abiotic stress tolerance under limiting water were analysed for sugar content and used for a transcript profiling study.

Sucrose accumulated in transgenic OsMADS6:OsT6PP maize plants, especially in leaf and floret. Please see the table below. The increase in sucrose accumulation indicates an increase in sink strength created by a pull on sucrose synthesis.

| | Event 1 versus Wild Type | | | | Event 2 versus Wild type | | | |
|---|---|---|---|---|---|---|---|---|
| | Time | Time | Time | Time | Time | Time | Time | Time |
| Sucrose | point 1 | point 2 | point 3 | point 4 | point 1 | point 2 | point 3 | point 4 |
| Leaf | 1.22 | 1.19 | 1.17 | 1.19 | 1.2 | 1.18 | 1.23 | 1.09 |
| Node | 1.03 | 1.24 | 1.29 | 1.12 | 0.96 | 1.29 | 1.11 | 1.03 |
| Pith | 1.17 | 0.71 | 0.65 | 0.88 | 1.18 | 1.32 | 0.46 | 0.77 |
| Shank | 0.96 | 0.7 | 1.12 | 1.11 | 0.92 | 0.74 | 1.07 | 1.11 |
| Floret | 1.15 | 1.11 | 0.93 | 1.08 | 1.1 | 1.16 | 0.92 | 1.22 |

Time point 1 = at silking/5 days before pollination
Time point 2 = at pollination
Time point 3 = 5 days after pollination
Time point 4 = 10 days after pollination

To identify the genes altered in transgenic OsMADS6: OsT6PP plants a transcript profiling study was initiated. ZmSWEET14b and ZmSWEET13b were significantly upregulated in abiotic stress tolerant transgenic maize expressing T6PP.

| | | | | |
|---|---|---|---|---|
| Fold increase | | | | |
| in expression | Log2FC | absFC | PValue | FDR |

T6PP Event 1

| | | | | | |
|---|---|---|---|---|---|
| ZmSWEET14b | 1.6133 | 0.690613 | 0.690613 | 4.99E−30 | 1.13E−27 |
| ZmSWEET13cδ | 1.8921 | 0.920767 | 0.920767 | 3.25E−21 | 3.99E−19 |

T6PP Event 2

| | | | | | |
|---|---|---|---|---|---|
| ZmSWEET14b | 1.4228 | 0.508759 | 0.508759 | 4.67E−17 | 8.24E−15 |
| ZmSWEET13cδ | 2.0272 | 1.019476 | 1.019476 | 1.21E−25 | 4.02E−23 |

In combination with the OsMADS6:T6PP transcript profiling study, a developmental study transcript profiling study was performed to understand the level of expression of different maize genes during development. Interestingly, ZmSWEET15b was identified as a primary embryo expressed and drought suppressed SWEET gene.

During drought stress at flowering, ZmSWEET15b is suppressed and contributes to less sugar being made during seed fill of the maize ear. Low sugar during seed fill has been shown to decrease seed yield in maize. Although not to be limited by theory, overexpression of ZmSWEET15b under an ovule/developing embryo specific promoter or a drought inducible embryo specific promoter could maintain an uninterrupted flow of sugar into the developing embryo and prevent drought induce embryo abortion.

### Example 2—Identification of Maize SWEET Gene Promoters

The tissue-specific expression of ZmSWEET14b, ZmSWEET13a and ZmSWEET15b were determined by an RNA sequencing strategy to comprehensively profile the mRNA populations in different maize tissues at different stages of development.

Below is a table with the counts which indicate the level of expression for each promoter in various maize tissues.

| DevStage.Tissue | ZmSWEET13a | ZmSWEET14b | ZmSWEET15b |
|---|---|---|---|
| V4.Nodal root | 1663.49 | 776.05 | 74.54 |
| V4.Seminal root | 1552.09 | 183.55 | 36.00 |
| V4.YFML - tip third | 16384.00 | 4.50 | 61.39 |
| V4.YFML - mid third | 16384.00 | 3.76 | 68.12 |
| V4.Shoot apical meristem | 263.20 | 93.70 | 455.09 |
| V7.YFML - tip third | 16384.00 | 0.50 | 17.51 |
| V7.YFML - mid third | 16384.00 | 6.23 | 23.75 |
| V7.YFML - base third | 15286.81 | 7.52 | 52.35 |
| V7.(YFML + 3) - tip | 16384.00 | 2.25 | 37.79 |
| V7.(YFML + 3) - middle | 5404.70 | 32.45 | 245.57 |
| V7.Primary Ear Shoot | 512.00 | 14.52 | 588.13 |
| V7.Elongating internode | 433.53 | 75.06 | 588.13 |
| V7.Tassel base/nodes | 146.02 | 153.28 | 227.54 |
| V7.Tassel | 59.30 | 22.94 | 256.00 |
| V9.Nodal root | 3326.99 | 776.05 | 48.84 |
| V9.Seminal root | 2521.38 | 132.51 | 19.29 |
| V9.Primary ear - whole | 195.36 | 87.43 | 724.08 |
| V9.Spikelets | 41.64 | 158.68 | 675.59 |
| V9.Anthers | 6.73 | 29.45 | 1097.50 |
| V11.YFML - tip third | 18820.27 | 1.49 | 21.71 |
| V11.YFML - mid third | 14263.10 | 3.51 | 20.25 |
| V11.YFML - base third | 9410.14 | 8.00 | 89.88 |
| V11.(YFML + 3) - tip | 16384.00 | 5.24 | 44.94 |
| V11.(YFML + 3) - middle | 10809.41 | 2.75 | 91.77 |
| V11.Primary ear - whole | 83.87 | 8.00 | 630.35 |
| V11.Tassel base/nodes | 385.34 | 464.65 | 388.02 |
| V11.Spikelets | 216.77 | 151.17 | 3326.99 |
| V11.Anthers | 4.50 | 4.99 | 5042.77 |
| V14.YFML - mid third | 17559.94 | 6.23 | 47.18 |
| V14.Ear Leaf - tip third | 17559.94 | 2.99 | 7.73 |
| V14.Ear Leaf - mid third | 18820.27 | 10.48 | 14.03 |
| V14.Ear Leaf - base third | 17559.94 | 4.26 | 26.17 |
| V14.Stem internode | 1663.49 | 166.57 | 47.50 |
| V14.Primary ear - shank | 315.17 | 588.13 | 354.59 |
| V14.Primary ear - whole | 66.26 | 103.97 | 233.94 |
| V14.Spikelets | 548.75 | 53.82 | 2194.99 |
| V14.Anthers | 28.05 | 24.93 | 5042.77 |
| VT.Nodal root | 1176.27 | 955.43 | 29.45 |
| VT.YFML - mid third | 17559.94 | 2.99 | 74.54 |
| VT.Ear Leaf - tip third | 20171.07 | 4.76 | 4.99 |
| VT.Ear Leaf - mid third | 18820.27 | 7.73 | 6.23 |
| VT.Ear Leaf - base third | 17559.94 | 4.00 | 13.27 |
| VT.Primary ear - shank | 508.46 | 776.05 | 259.57 |
| VT.Tassel base/nodes | 6653.97 | 173.65 | 98.36 |
| VT.Spikelets | 1351.18 | 83.87 | 4389.98 |
| VT.Pollen | 109.14 | 18.25 | 588.13 |
| R1-5d.Ear Leaf - mid third | 18820.27 | 9.25 | 4.99 |
| R1-5d.Primary ear node (pith) | 891.44 | 203.66 | 390.72 |
| R1-5d.Primary ear - shank | 413.00 | 891.44 | 266.87 |
| R1-5d.Primary ear - whole | 72.00 | 99.73 | 243.88 |
| middle and tip with silks | | | |
| R1-2d.Ear Leaf - mid third | 18820.27 | 29.45 | 11.47 |
| R1-2d.Primary ear - shank tip | 487.75 | 512.00 | 198.09 |
| R1-2d.Primary ear - shank middle | 776.05 | 1260.69 | 171.25 |
| R1-2d.Primary ear - shank base | 455.09 | 630.35 | 150.12 |
| R1-2d.Cob - primary ear tip | 218.27 | 675.59 | 491.14 |
| R1-2d.Cob - primary ear - middle | 272.48 | 891.44 | 501.46 |
| R1-2d.Primary ear - Ovules - ear tip | 101.13 | 630.35 | 315.17 |
| R1-2d.Primary ear - Ovules - mid-ear | 112.21 | 1260.69 | 436.55 |
| R1-2d.Primary ear - Silks | 128.89 | 13.00 | 548.75 |
| R1.Ear Leaf - mid third | 18820.27 | 12.47 | 5.50 |
| R1.Stem internode | 2352.53 | 186.11 | 22.78 |
| R1.Primary ear - shank tip | 1351.18 | 1097.50 | 254.23 |
| R1.Primary ear - shank middle | 1024.00 | 831.75 | 181.02 |
| R1.Primary ear - shank base | 675.59 | 776.05 | 163.14 |
| R1.Cob - primary ear tip | 675.59 | 1663.49 | 1260.69 |
| R1.Cob - primary ear - middle | 1097.50 | 4096.00 | 1176.27 |
| R1.Primary ear - Ovules - ear tip | 224.41 | 1552.09 | 1097.50 |
| R1.Primary ear - Ovules - mid-ear | 306.55 | 2352.53 | 1663.49 |
| R1.Primary ear - Silks | 155.42 | 4.26 | 1351.18 |

The ZmSWEET14b promoter (SEQ ID NO: 33) drives significant expression of a gene in the sink tissues, specifically in the ear and ovules.

The ZmSWEET13a promoter (SEQ ID NO: 32) drives significant expression of a gene in vegetative and reproductive tissues and at all developmental stages. The promoter drives high expression in bundle sheath and vascular tissues.

The ZmSWEET15b promoter (SEQ ID NO: 34) drives expression in the tissues of a developing maize kernel, in particular expression of a gene in the embryo In addition, the expression pattern of ZmSWEET15b suggests that in addition to high expression in the embryo, the promoter is drought suppressed.

### Example 3—Example Constructs

Constructs including at least one SWEET 13 protein, SWEET 14 protein, SWEET 15 and/or T6PP polynucleotide operably linked to a promoter, such as an OsMAD6 or OsMADS7 promoter or a Zea mays native promoter (e.g., a Zm SWEET 13, 14 or 15 native promoter), will be prepared. Some constructs may include two SWEET proteins (e.g., two SWEET 13 proteins, two SWEET 14 proteins, two SWEET 15 proteins, or one SWEET 13 protein, one SWEET 14, and one SWEET15 protein or any combination thereof). Some constructs may include at least one SWEET protein (e.g., a SWEET 13 protein, SWEET 14 protein or SWEET 15 protein) and at least one T6PP protein. Example constructs are provided below.

Promoter+SWEET 13a

Promoter+SWEET 13c

Promoter+SWEET 13c6

Promoter+SWEET 14b

Promoter+SWEET 15b

Promoter+SWEET 13a & Promoter+SWEET 13c

Promoter+SWEET 13a & Promoter+SWEET 13c6

Promoter+SWEET 13a & Promoter+SWEET 14b

Promoter+SWEET 13c & Promoter+SWEET 13c6

Promoter+SWEET 13c & Promoter+SWEET 14b

Promoter+SWEET 13c6 & Promoter+SWEET 14b

Promoter+SWEET 13c & Promoter+SWEET 15b

Promoter+SWEET 13c6 & Promoter+SWEET 15b

Promoter+SWEET 14b & Promoter+SWEET 15b

Promoter+T6PP & Promoter+SWEET 13a

Promoter+T6PP & Promoter+SWEET 13c

Promoter+T6PP & Promoter+SWEET 13c6

Promoter+T6PP & Promoter+SWEET 14b

Promoter+T6PP & Promoter+SWEET 15b

Promoter+T6PP & Promoter+SWEET 13a & Promoter+SWEET 13c

Promoter+T6PP & Promoter+SWEET 13a & Promoter+SWEET 13c6

Promoter+T6PP & Promoter+SWEET 13a & Promoter+SWEET 14b

Promoter+T6PP & Promoter+SWEET 13c & Promoter+SWEET 13c6

Promoter+T6PP & Promoter+SWEET 13c & Promoter+SWEET 14b

Promoter+T6PP & Promoter+SWEET 13c6 & Promoter+SWEET 14b

Specific example constructs for overexpression in maize include:

OsMADS6 promoter: ZmSWEET14b
OsMADS6 promoter: ZmSWEET13a
OsMADS6 promoter: ZmSWEET13c6
OsMADS6 promoter: ZmSWEET13c
OsMADS6 promoter: ZmSWEET15b
OsMADS7 promoter:ZmSWEET15b
ZmSWEET13a promoter: ZmSWEET13a
ZmSWEET13a promoter: ZmSWEET13c6
ZmSWEET13a promoter: ZmSWEET13c
ZmSWEET14b promoter: ZmSWEET14b
ZmSWEET15b promoter: ZmSWEET15b
MN1 [miniature 1] promoter: ZmSWEET13c
OsMADS6 promoter:OsT6PP & OsMADS6:ZmSWEET14b
OsMADS6 promoter:OsT6PP & ZmSWEET14b:ZmSWEET14b
OsMADS6 promoter:OsT6PP & ZmSWEET13a:ZmSWEET13c
OsMADS6 promoter: OsT6PP [or T6PP variant] & OsMADS6 promoter: ZmSWEET14b
ZmSWEET14b promoter: OsT6PP [or T6PP variant] & ZmSWEET14b promoter: ZmSWEET14b
OsMADS6 promoter: OsT6PP-01 & ZmSWEET14b promoter: ZmSWEET14b
OsMADS6 promoter: dN56OsT6PP-01 [N-terminal truncated] & ZmSWEET14b promoter: ZmSWEET14b

Specific example constructs for overexpression in sugar cane include:

Stem specific promoter: OsT6PP [or T6PP variant] & Stem specific promoter: Sucrose Synthase
Stem specific promoter: OsT6PP [or T6PP variant] & Stem specific promoter: Sucrose Isomerase [Isomultulase]
Stem specific promoter: OsT6PP [or T6PP variant] & Stem specific promoter: Ketose Synthase
Stem specific promoter: OsT6PP [or T6PP variant] & Stem specific promoter: Sucrose transporter

### Example 4—Vector Construction for Transformation into Corn

Vectors will be constructed and transformed into corn as described in U.S. Pat. No. 8,129,588, the contents of which are incorporated herein by reference in their entirety. T-DNA insertion will be confirmed by primary and secondary TaqMan analysis using several target assays that span the T-DNA insert and the binary vector backbone. Events lacking a vector backbone signal will be retained. The integrity of the gene of interest and phosphomannose isomerase (PMI, transformation marker) protein coding sequence in selected events will be confirmed as identical to the transformation vector sequence.

### Example 5—Vector Constructions for Transformation into Sugar Cane

### Plant Materials

Sugar cane materials will be L97-128 (kindly provided by Dr. Kenneth Gravois, Louisiana State University), CP84-1198 (kindly provided by the Canal Point USDA Sugar Cane Breeding Station), and SP70-1143 (kindly provided by Sugar Cane World Collection in Coral Gables, Fla.) grown at the Syngenta Biotechnology Inc, Cornwallis Rd. location, Research Triangle Park, N.C. Sugar cane tops from immature tillers containing the immature leaf whorl will be collected and initiated into tissue culture within 3 hours of harvest, essentially as described by Bower and Birch (Bower R, Birch R G (1992) Transgenic sugarcane plants via microprojectile bombardment. The Plant Journal 2: 409-416). Cultures will be maintained in the dark at 27° C.±1° C. and sub-cultured onto fresh media every 12 to 14 days for a period of 28 to 42 days. Embryogenic calli will be selected as target tissue for transformation providing consistent transformation and high frequency regeneration.

Preparation of Agrobacterium and Infection and Co-Cultivation Agrobacterium cultures harboring the selectable marker gene, PMI, and scorable marker gene Amcyan (licensed from Clontech Laboratory, Inc.) will be streaked onto Luria Bertani medium containing the appropriate antibiotics and grown at 28° C. for 3 days. Prior to transformation, a single colony streak onto a fresh LB plate is grown for 1 to 2 days at 28° C. and used to inoculate a liquid culture of Agrobacterium strain EHA101 (modified from Khanna, et al. 2004). The density of the bacterial cell suspension will be measured using a spectrophotometer and the Agrobacterium will be diluted to OD660 of 0.2 to 0.5. To induce virulence gene expression, the Agrobacterium will be incubated in inoculation medium containing acetosyringone with shaking for 0.5 to 4 hours in darkness.

The sugar cane embryogenic calli will be heat shocked at 45° C. for 5 minutes in a 50 ml of inoculation medium. The medium will then be drained from the callus tissue, and 25 to 30 ml of the Agrobacterium inoculation suspension is added to each tube and mixed gently. The mixture will be incubated in the dark for approximately 10 minutes with gentle rotation at room temperature. Then, the mixture will be sonicated for 2 minutes, followed by 10 minutes incubation. The Agrobacterium suspension will then be drained from the calli and the remaining culture is blotted dry to remove excess Agrobacterium suspension. The calli will then be transferred to petri dishes. The dishes will be sealed with plastic film for co-cultivation in the dark at 22° C. for 2 to 3 days.

Post-Transformation and Regeneration and Selection

Following co-cultivation the callus material will be allowed to recover by transferring to embryogenic calli culture medium containing 200 mg/L of Timentin antibiotic (GlaxoSmithKline Inc.) and keeping in the dark at 28° C. for a period of 4 to 7 days. Selection will be carried out in medium containing 200 mg/L of Timentin antibiotic for 28 days in the dark at 28° C. Regeneration will be conducted on SC-BAP medium (Murshige and Skoog salts, B5 vitamins, 30 g/L sucrose, 7 g/L phylablend agar, 2 mg/L benzylaminopurine, mannose and 200 mg/L Timentin antibiotic) 27° C. in 16 hours light. For the first week, the culture will be left at low light intensity, and for the next 3 weeks, the culture will be grown at moderate light intensity. Shoot formation will be observed between the second and fourth weeks. When leaves appear, the shoots are transferred to SC-MS medium (Murshige and Skoog salts, B5 vitamins, 30 g/L sucrose, 3 g/L Phytagel agar, 6 mg/L mannose and 200 mg/L Timentin antibiotic) until the plants are 4 to 5 cm in height. The plants will be transferred to containers with rooting media. The plantlets will then be sampled for TaqMan analysis to confirm events containing a transgene insertion and estimate copy number. Transgene positive plants with low copy number will be selected, transferred to soil and placed in the greenhouse to grow to maturity.

### Example 6—Transgenic Plant Assays

Selected transgenic plants from Example 2 and/or 3 will be tested for the following:
for expression of the gene of interest (GOI), selectable markers,
for expression of target transcripts [using Fluidigm assay] related to trehalose signaling, stress management, sugar transport and metabolism in various corn tissue samples,
for level of targeted metabolites associated with trehalose signaling, sucrose metabolism and stress management in various tissues, such as sink tissues (e.g., flowering tissues)
for changes in yield components [anthesis silking interval (ASI), kernel number, kernel row, kernel weight, etc.] under managed water deficit during flowering and vegetative stages of corn development

### Example 7—Metabolite Testing of Transgenic Plants

The concentration of various metabolites will be estimated in selected transgenic plants from Example 2 and/or 3 using the following procedures.

### Glucose, Fructose and Sucrose Estimation

For sucrose, sugars soluble in 80% ethanol will be extracted at room temperature from powdered tissue samples. Four to six samples each from wild type control plants and transgenic events will be analyzed. Approximately 100 mg of tissue will be weighed and vortexed in 500 μL 80% ethanol solution for 5 minutes at room temperature. Samples will then be clarified by centrifugation at 15700×g for 10 min at room temperature in a bench-top centrifuge. The collected supernatants will be centrifuged again and then filtered through a MicroScreen-HV plate (Millipore, Catalog No. MAHVN4550). All filtered samples will be diluted 50-fold with water before chromatographic analysis.

A Dionex ICS-3000 Ion Chromotography System equipped with a CarboPac PA1 column will be used to resolve glucose, fructose and sucrose in each sample. Sugars will be separated with a 35 minute elution gradient [40 mM NaOH for 25 minutes, followed by a 0-300 mM sodium acetate gradient in 40 mM NaOH for 1 minute, and then 40 mM NaOH for 9 minutes] at a flow rate of 1 mL/min. Sucrose, glucose and fructose will be quantified by determining resolved peak areas using Chromeleon software and comparing to standard curves generated in the concentration range of 0.0125 to 0.2 mg/mL. Three measurements will be done for each tissue sample and data are the mean±standard error (n=4).

### Synthesis of ¹³C-Labeled UDP-Glucose, Trehalose 6-Phosphate and Sucrose 6-Phosphate Internal Standards

Uridine-5′-diphospho glucose (UDP-Glc)-¹³C₉ is synthesized via an enzymatic reaction using uridine-5′-diphosphoglucose pyrophosphorylase (Sigma-Aldrich U8501) with the substrates glucose-1-phosphate (G1P, Sigma-Aldrich G6750)+¹³C9-labeled uridine-5′-triphosphate (¹³C9-UTP, Sigma-Aldrich G6750). 10 mg G1P, 5 mg ¹³C9-1UTP, 25 units uridine-5′-diphosphoglucose pyrophosphorylase, and 100 units inorganic pyrophosphatase (Sigma-Aldrich 11643) are mixed in 50 mM TRIZMA buffer pH 7.6 with 16 mM magnesium chloride (Sigma-Aldrich M2670). Reaction progress is monitored by LC-MS/MS, and the reaction is quenched with methanol when no further progression is detected. Based on peak area comparison with an unlabeled UDP-Glc standard, 1.4 mL of solution containing approximately 800 ag/mL UDP-Glc-¹³C₉ is obtained.

Trehalose-6-phosphate (T6P)-¹³C₁₂ is synthesized via a water-mediated phosphorus oxychloride reaction with ¹³C₁₂-trehalose (Omicron Biochemicals TRE-002). ¹³C₁₂-trehalose (100 mg) is added to 0.5 mL acetonitrile at 4° C. and mixed with phosphorus oxychloride (Sigma-Aldrich 262099) and a small amount of water. The reaction is monitored by LC-MS/MS, which showed a mixture of products, including T6P-¹³C₁₂. The reaction is quenched with water when the maximum amount of T6P-¹³C₁₂ is indicated. The resultant 1.5 mL of solution contained approximately 6.6 mg/mL T6P-¹³C₁₂, based on peak area comparison with an unlabeled T6P standard.

Sucrose-6-phosphate (Suc-6P)-¹³C₁₂ is synthesized via a water-mediated phosphorus oxychloride reaction with ¹³C₁₂-sucrose. ¹³C₁₂-sucrose (100 mg) is added to 0.5 mL acetonitrile at 4° C. and mixed with phosphorus oxychloride and a small amount of water. The reaction is monitored by LC-MS/MS, which showed a mixture of products, including S6P-¹³C₁₂. The reaction is quenched with water when the maximum amount of Suc-6P-¹³C₁₂ is indicated. The resultant 5 mL of solution contained approximately 780 ag/mL of Suc6P-¹³C₁₂, as estimated from peak area and comparison with an unlabeled S6P standard.

The internal standard solutions are mixed to obtain an internal standard working solution 1 containing UDP-Glc-¹³C₉ at 20 ag/mL, T6P-¹³C₁₂ at 15 ag/mL, and Suc-6P-¹³C₁₂ at 25 ag/mL. 1 mg/ml stock solutions are separately prepared, in water, for Glc6P (Glc)-¹³C₆ and trehalose (Tre)-¹³C₁₂. These two stock solutions are combined and diluted with methanol:water (80:20) to produce a working internal standard solution 2 containing Glc6P-¹³C₆ at 50 ag/mL and Tre-¹³C₁₂ at 20 ag/mL.

### T6P, Suc-6P, UDP-Glc, Glc-6P and Trehalose Estimation

Powdered maize tissue sample (e.g. floret approximately 100 mg) will be spiked with working ¹³C-labeled internal standard solution, then homogenized and extracted with methanol water (70:30). Homogenization and extraction of tissue samples will be performed with a Genogrinder homogenization device. Following centrifugation, an aliquot of the clear supernatant will be removed, and injected onto an Agilent 1290/AB Sciex QTrap-5500 LC MS MS system equipped with an Acquity Amide UPLC column [Acquity BEH-Amide, 1.7 micron, 2.1×100 mm, Waters].

For estimation of T6P, Suc-6P, and UDP-Glc, 2 μL [adjusted for sensitivity/linearity purposes] of sample with internal standard 1 will be injected. Chromatography will be carried out with 35% mobile phase A (200 mM ammonium bicarbonate in water) and 65% mobile phase B (acetonitrile) isocratic flow at 0.800 mL/min.

Samples with internal standard solution 2 will be used for estimation of Glc-6P and trehalose contents. Chromatography through the Acquity Amide UPLC column will be carried out with gradient flow at 0.40 mL/min for a total time of 7.0 min. Two mobile phases are: A, containing 200 mM ammonium formate with 0.5% ammonium hydroxide in water and B, containing 9:1 acetonitrile:methanol. The gradient used is: 0.0 min 5% mobile phase A and 95% mobile phase B; 3.9 min 28% mobile phase A and 72% mobile phase B; 4.4 min 50% mobile phase A and 50% mobile phase B; 6.6 min 5% mobile phase A and 95% mobile phase B. For maize tissue samples, the peak areas of the m/z 421.0→240.9 product ion of T6P, the m/z 421.0→240.9 product ion of Suc-6P, the m/z 259.0→139.0; 169.0; and 199.0 product ions of G6P, the m/z 564.9→240.9 product ion of UDP-Glc, and the m/z 341.2→59.0; 179.0; 89.1; and 119.0 product ions of trehalose will be measured against the peak areas of the corresponding internal standard product ions of m/z 433.0→246.9, m/z 433.0→246.9, m/z 265.0→141.0; 172.0; 203.0, m/z 573.9→240.9, and m/z 353.2→61.0; 185.0; 92.0; 123.0.

The following LC-MS/MS methods are developed with a calibration range of 10.0 to 1000 ag/g for Glc-6P, 0.0500 to 50.0 ag/g for T6P, 0.500 to 500 ag/g for Suc-6P, 0.500 to 500 ag/g for UDP-Glc, and 0.500 to 50.0 ag/g for trehalose. The peak areas of the T6P, Suc-6P, Glc-6P, UDP-Glc, and trehalose product ions will be measured against the peak area of the respective T6P-13C12, Suc-6P-13C12, Glc-6P-13C6, UDP-Glc-13C9, and trehalose-13C12, internal standard product ions. Quantitation will be performed using a weighted linear least squares regression analysis generated from fortified calibration standards prepared immediately prior to analysis.

### Example 8: Assay of Plants Expressing SWEET and/or T6PP in Flowering Tissue

The following vectors are transformed into maize using the methods described above in order to test abiotic stress tolerance of maize plants in either a greenhouse or in a field assay:

| | Promoter | Gene | Terminator | Promoter 2 | Gene 2 | Terminator 2 |
|---|---|---|---|---|---|---|
| 1 | OsMADS6 | ZmSWEET14b | OsMADS6 | | | |
| 2 | OsMADS6 | ZmSWEET13a | OsMADS6 | | | |
| 3 | OsMADS6 | ZmSWEET13cδ | OsMADS6 | | | |
| 4 | OsMADS6 | ZmSWEET13a | OsMADS6 | OsMADS6 | ZmSWEET14b | OsMADS6 |
| 5 | ZmSWEET13a | ZmSWEET13a | ZmSWEET13a | | | |
| 6 | ZmSWEET14b | ZmSWEET14b | ZmSWEET14b | | | |
| 7 | ZmSWEET15b | ZmSWEET15b | ZmSWEET15b | | | |
| 8 | OsMADS7 | ZmSWEET15b | OsMADS7 | | | |
| 10 | OsMADS6 | OsT6PP | OsMADS6 | | | |

In addition, a drought inducible embryo specific promoter could be operably linked to a ZmSWEET15b to ensure expression tissue specific expression of ZmSWEET15b even under drought stress conditions.

To create maize plants comprising one or more SWEET genes alone or in combination with a T6PP gene, molecular stacks comprising one or more genes can be transformed into a plant. For example, an expression vector can be created containing both a SWEET13a polynucleotide and a SWEET14b polynucleotide. This expression vector can then be transformed into a plant to create a transgenic plant expressing both a SWEET13a polypeptide and a SWEET 14b polypeptide. Alternatively, a transgenic plant comprising one or more SWEET and/or T6PP genes can be crossed with a transgenic plant comprising one or more SWEET and/or T6PP genes with the resulting offspring comprising a breeding stack of multiple SWEET genes alone or in combination with OsT6PP.

Seed containing the OsMADS6 promoter operably linked to the SWEET14b polynucleotide (Construct number 1 in the table above) was used to look at the effect of the transgene on yield under water stress and well watered conditions in a greenhouse. Each plant was tested for the presence of the transgene and to confirm zygosity. Trait gene expression was confirmed by qRT-PCR. Null plants were included as controls. The irrigation management protocol used achieves an approximately 45% reduction in both kernel number and grain weight of control plants. To achieve this level of water stress, plants were first grown in pots with appropriate potting medium until ear shoots of 70% of the plants are between 0.5 and 1 inch. Using soil moisture sensors, the soil is allowed to dry out to a 32% soil moisture point. Low moisture in the pot is maintained for 15 days after the water stress was initiated. Normal irrigation is then resumed until about 10 days before harvest.

In addition, subsets of null and transgenic plants were not subject to water stress and were considered the “well watered control”.

The following yield component data was collected during the experimental protocol:
Record pollen shed and silking date daily during flowering stage.
Plant height and ear height before harvest
Upon trial harvest, ears are air-dried for 7-10 days
Ear length and kernel row number
Grain weight/ear are recorded and kernels/ear are counted
Seed moisture was determined using near-infrared spectroscopy (NIR)

The transgenic plants expressing SWEET4b and null plants were analyzed for anthesis and silking under water stress and well watered conditions. In general, water stress delayed silking by three to four days for both transgenic and null plants. There was no significant difference between null and transgenic plants for anthesis or silking.

Yield components (kernel number, grain moisture and grain weight) were recorded after harvest. Grain weight was adjusted to standard grain moisture of 15.5%.

| | | qRT | | Kernel No./ear | | | Grain weight (gram/ear) | | |
|---|---|---|---|---|---|---|---|---|---|
| Treatments | Event# | V9 | Reps | Het | Null | p-value | Het | Null | p-value |
| Well | 1 | 135 | 5 | 391 | 358 | 0.0383 | 95.6 | 84.8 | 0.0347 |
| watered | 2 | 34 | 5 | 467 | 410 | 0.1798 | 110.2 | 105.3 | 0.5895 |
| Water | 1 | 135 | 15 | 180 | 196 | 0.4758 | 38.9 | 42.1 | 0.5228 |
| stress | 2 | 34 | 15 | 216 | 179 | 0.4998 | 46.4 | 31.9 | 0.0515 |

Under well watered conditions, transgenic plants from both events showed positive effects on kernel number and grain weight compared to their segregated nulls. The trait efficacy of event 1 on kernel number and grain weight showed statistically significant (p-value=0.0383 and 0.0347 respectively).

Under well watered conditions event 1 increased kernel number and grain weight per ear by 8.5% and 11.3% respectively, compared to segregated null. Event 2 increased kernel number and grain weight per ear by 12.2% and 4.5% respectively, compared to segregated null. Under water stress conditions there was no penalty on kernel number and grain weight in either event. Event 2 showed significantly positive effects on kernel number and grain weight compared to the segregated null.

The expression of SWEET4b in flowering tissue resulted in improved yield under both well watered and water stress conditions.

### Example 9: Testing Constructs in a Corn Cob Transient Assay

Maize ears were harvested from the greenhouse 3-5 days after pollination (DAP). The ears were sterilized by spraying with 70% alcohol before removing most of the husks. The remaining 2-3 husks were removed in a sterile laminar-flow hood. The clean cob was either cut (cross-section) into 1-2 mm slices using a sterile scalpel or the pith was isolated by removing the kernels and then slicing the pith into small circular pieces (1-2 mm).

Preparation of Agrobacterium cultures was carried out as described by Azhakanandam et al., Plant Mol. Biol. 63: 393-404 (2007) with modification. In brief, Agrobacterium containing test constructs from glycerol stock were streaked on a fresh YP plate with appropriate antibiotics for initial growth and kept at 28° C. for 1-2 days. The agrobacteria were re-streaked densely on a fresh YP plate about 16 hours before co-cultivation with cob tissues. Agrobacteria from YP plate was collected using a sterile loop and re-suspended in 10-15 mL infection medium ([Murashige and Skoog salts with vitamins, 2% sucrose, 500 μM MES (pH 5.6), 10 μM MgSO4, and 100 μM acetosyringone] in a 50 mL plastic tube by vortexing for about 30-60 seconds. Bacterial concentration was checked and adjusted to OD600=1.0 and the required volume for experiment was prepared. The bacterial suspension was kept at 28° C. for about an hour prior to co-cultivation. Prior to co-cultivation 0.02% Silwet was added to the bacterial cultures. Twenty five ml of bacterial culture was used for treatment/construct

The sliced corn cob/pith explants were co-cultivated with Agrobacterium for 30 mins followed by removing the Agrobacterium suspension and transferring the explants to the sterile filter paper briefly to remove excess of bacteria. The explants were then quickly transferred to modified semi-solid co-culture medium (Li et al., 2013) and kept for 5 days in a dark chamber at 23 C. After 5 d days of co-cultivation, the explants were harvested for qRT-PCR, Metabolomic profiling and Fludigm analysis.

The following constructs will be tested in the Corn Cob assay:

| GENE | TRAIT CASSETTE |
|---|---|
| ZmSWEET13a | prOsMADS6:ZmSWEET13a:tOsMADS6 |
| ZmSWEET13c | prOsMADS6:cZmSWEET13c:tOsMADS6 |
| ZmSWEET14b | prOsMADS6:ZmSWEET14b:tOsMADS6 |
| OsT6PP, | prOsMADS6:OsT6PP:tOsMADS6; |
| ZmSWEET14b | prZmSWEET14:ZmSWEET14b:tOsMADS6 |
| OsT6PP-01 N56del, | prOsMADS6:OsT6PP with N56 |
| ZmSWEET14b | deletion:tOsMADS6; |
| | prZmSWEET14b:ZmSWEET14b:tZmSWEET14b |
| OsT6PP | prOsMADS6:OsT6PP:tOsMADS6 |

The transformed tissues will be analyzed for the level of sugars as described in Nuccio, et. al. (2015) Nature Biotechnology, Volume: 33, Pages: 862-869.

It is expected that the expression of the transgene will lead to increased sugar accumulation. In Boyer J S, and Westgate M E J. Exp. Bot. 2004; 55:2385-239, prolonged drought decreases carbon assimilation in source tissues and reduces supply of sucrose to the sink. This can be specifically reversed by sucrose infusion. Therefore, overexpression of a SWEET gene is expected to increase sugar and thereby function in a similar way to sucrose infusion.

The foregoing is illustrative of the present invention and is not to be construed as limiting thereof. Although a few exemplary embodiments of this invention have been described, those skilled in the art will readily appreciate that many modifications are possible in the exemplary embodiments without materially departing from the novel teachings and advantages of this invention. Accordingly, all such modifications are intended to be included within the scope of the present invention.

1. A method of increasing yield stability under drought conditions in a plant, comprising:
expressing in the plant one or more exogenous nucleic acids selected from the group consisting of:
(a) a Clade III sugar transporter nucleic acid comprising SEQ ID NO: 25
(b) a SWEET 13 nucleic acid comprising SEQ ID NO:25;
(c) a SWEET 14 nucleic acid comprising SEQ ID NO:25;
(d) a SWEET 15 nucleic acid comprising SEQ ID NO:25;
(e) a nucleic acid having a nucleotide sequence set forth in any one of SEQ ID NOs:1 to 11, 29 to 32;
(f) a nucleic acid which is at least 70% identical to the nucleotide sequence of any one of SEQ ID NOs:1 to 11, 29 to 32;
(g) a nucleic acid that encodes a polypeptide comprising the amino acid sequence set forth in any one of SEQ ID NOs:12 to 16, 33 to 34;
(h) a nucleic acid that encodes a polypeptide comprising an amino acid sequence that is at least 70% identical to the amino acid sequence set forth in any one of SEQ ID NOs:12 to 16, 33 to 34; and
(i) a nucleic acid that hybridizes to the nucleic acid of any one of (d) to (g) above under stringent hybridization conditions;
wherein expression of the exogenous nucleic acid results in increased yield stability under drought conditions in the plant as compared to a control plant.

2. The method of claim 1, wherein the exogenous nucleic acid is operably linked to a tissue-specific promoter sequence, optionally a flower-, seed-, endosperm-, embryo-, panicle-, and/or node-specific promoter sequence.

3. The method of claim 2, wherein the promoter provides expression of the exogenous nucleic acid in ear node, ear vasculature and spikelet tissue.

4. The method of claim 1, wherein the exogenous nucleic acid is operably linked to a promoter is selected from the group consisting of a drought inducible promoter, a drought inducible embryo specific promoter and a drought inducible reproductive tissue preferred promoter.

5. The method of claim 2, wherein the promoter is an OsMADS promoter.

6. The method of claim 5, wherein the promoter is an OsMADS6 or OsMADS 7 promoter.

7. The method of claim 1, wherein the exogenous nucleic acid is operably linked to a promoter selected from the group consisting of a SWEET 13 promoter, a SWEET 14 promoter or a SWEET 15 promoter.

8. The method of claim 7, wherein the promoter is selected from the group consisting of SEQ ID NO: 32, SEQ ID NO: 33 and SEQ ID NO: 34.

9. The method of claim 1 further comprising an additional exogenous nucleic acid encoding a trehalose phosphate phosphatase.

10. The method of claim 9, wherein the trehalose phosphate phosphatase comprises a nucleotide sequence that is at least 70% identical to the nucleotide sequence of any one of SEQ ID NOs:17 to 20 and/or a nucleotide sequence that is at least 70% identical to a nucleotide sequence that encodes a polypeptide comprising the amino acid sequence of any one of SEQ ID NOs:21 to 24.

11. The method of claim 1, further comprising one or more exogenous nucleic acid selected from the group consisting of:
an exogenous nucleic that encodes a gene product that provides enhanced abiotic stress resistance, optionally enhanced drought stress tolerance, enhanced osmotic stress tolerance, enhanced salt stress tolerance and/or enhanced temperature stress tolerance;
an exogenous nucleic acid that encodes a gene product that provides resistance to—one or more herbicides, optionally glyphosate-, sulfonylurea-, imidazolinione-, dicamba-, glufisinate-, phenoxy proprionic acid-, cycloshexome-, traizine-, benzonitrile-, and/or broxynil-resistance;
an exogenous nucleic acid that encodes a gene product that provides resistance to one or more pests, optionally Acarina, bacterial, fungal, gastropod, insect, nematode, oomycete, phytoplasma, protozoa and/or viral resistance; and
an exogenous nucleic acid that encodes a gene product that provides resistance to one or more plant diseases.

12. The method of claim 1, wherein the plant is a monocot, optionally rice, maize, wheat, sorghum, or sugar cane.

13. The method of claim 1, wherein the plant is a dicot, optionally cotton, soybean, sugar beet, sunflower, tobacco, Brassica spp or tomato.

14. An expression cassette comprising one or more exogenous nucleic acids operably linked to a promoter, wherein the exogenous nucleic acid is selected from the group consisting of:
a) a Clade III sugar transporter nucleic acid comprising SEQ ID NO: 25
b) a SWEET 13 nucleic acid comprising SEQ ID NO:25;
c) a SWEET 14 nucleic acid comprising SEQ ID NO:25;
d) a SWEET 15 nucleic acid comprising SEQ ID NO:25;
e) a nucleic acid having a nucleotide sequence set forth in any one of SEQ ID NOs:1 to 11, 29 to 32;
f) a nucleic acid which is at least 70% identical to the nucleotide sequence of any one of SEQ ID NOs:1 to 11, 29 to 32;
g) a nucleic acid that encodes a polypeptide comprising the amino acid sequence set forth in any one of SEQ ID NOs:12 to 16, 33 to 34;
h) a nucleic acid that encodes a polypeptide comprising an amino acid sequence that is at least 70% identical to the amino acid sequence set forth in any one of SEQ ID NOs:12 to 16, 33 to 34;
i) a nucleic acid that hybridizes to the nucleic acid of any one of (d) to (g) above under stringent hybridization conditions;
and further comprising a exogenous nucleic acid encoding a trehalose-6-phosphate phosphatase operably linked to a promoter.

15. The expression cassette of claim 14, wherein at least one promoter comprises a tissue-specific promoter sequence, optionally a flower-, seed-, endosperm-, embryo-, panicle-, and/or node-specific promoter sequence.

16. The expression cassette of claim 14, wherein at least one promoter comprises a promoter is selected from the group consisting of a drought inducible promoter, a drought inducible embryo preferred promoter and a drought inducible reproductive tissue preferred promoter.

17. The expression cassette of claim 14, wherein at least one promoter comprises a promoter selected from the group consisting of a SWEET 13 promoter, a SWEET 14 promoter and a SWEET 15 promoter.

18. The expression cassette of claim 17, wherein at least one promoter comprises a promoter selected from the group consisting of SEQ ID NO: 32, SEQ ID NO: 33 and SEQ ID NO: 34.

19. A transgenic plant or plant part comprising the expression cassette of claim 14.

20. The transgenic plant or plant part of claim 19, wherein the plant or plant part is a monocot, optionally rice, maize, wheat or sugar cane.

21. A product harvested from the transgenic plant or plant part of claim 19.

22. A processed product produced from the harvested product of claim 21.

23. A crop comprising a plurality of the transgenic plant of claim 19.

24. A use of the transgenic plant of claim 19 for increasing yield, increasing yield preservation, and/or enhancing drought tolerance.

25. A use of the expression cassette of claim 14 for increasing yield, increasing yield preservation, and/or enhancing drought tolerance in a plant or plant part.

26. (canceled)
